(19) United States
(12) Patent Application Publication
(10) Pub. No.: US 2021/0147814 A1
(43) Pub. Date: May 20, 2021

(54) METHODS FOR GENERATING PLURIPOTENT STEM CELLS

(71) Applicant: Memorial Sloan Kettering Cancer Center, New York, NY (US)

(72) Inventors: Kitai Kim, New York, NY (US); Maria Skamagki, New York, NY (US)

(73) Assignee: Memorial Sloan Kettering Cancer Center, New York, NY (US)

(21) Appl. No.: 16/630,420

(22) PCT Filed: Jul. 12, 2018
(86) PCT No.: PCT/US2018/041851

§ 371 Date: Jan. 10, 2020

Related U.S. Application Data
(60) Provisional application No. 62/531,672, filed on Jul. 12, 2017.

(51) Int. Cl. C12N 5/074; A61K 35/12

(52) U.S. Cl. CPC C12N 5/0696; C12N 2500/30; A61K 35/12

(57) ABSTRACT

The present technology relates generally to the generation of induced pluripotent stem cells (iPSCs). In particular aspects, the present technology relates generally to methods for generating iPSCs from non-pluripotent cells, such as aged somatic cells, wherein the iPSCs are characterized by improved genomic stability, improved DNA damage response, increased ZSCAN10 expression, reduced GSS expression, and/or increased reprogramming efficiency.

### CROSS-REFERENCE TO RELATED APPLICATIONS

This application claims the benefit of and priority to U.S. Provisional Patent Application No. 62/531,672, filed Jul. 12, 2017, the entire contents of which are incorporated herein by reference.

### STATEMENT OF FEDERALLY FUNDED RESEARCH

This invention was made with government support under AG043531 awarded by the National Institutes of Health. The government has certain rights in the invention.

### TECHNICAL FIELD

Disclosed herein are methods related to the generation of induced pluripotent stem cells (iPSCs). The present technology relates generally to methods for generating iPSCs from non-pluripotent cells, such as aged somatic cells, wherein the iPSCs are characterized by improved genomic stability, improved DNA damage response, increased ZSCAN10 expression, reduced glutathione synthetase (GSS) expression, and/or increased reprogramming efficiency.

### BACKGROUND

The following description is provided to assist the understanding of the reader. None of the information provided or references cited is admitted to be prior art.

Induced pluripotent stem cells (iPSCs) hold enormous potential for generating histocompatible transplantable tissue using a patient's own somatic cells. While older patients are more likely to suffer from degenerative diseases and benefit from iPSC-based therapies, both basic and clinical researchers have reported mitochondrial and genomic mutations or instability of iPSC generated from aged donor tissue (A-iPSC). A clinical trial reported two cases of transplantation of retinal pigment epithelium (RPE) differentiated from autologous iPSC to treat age-related macular degeneration (AMD). Although A-iPSC-derived RPE successfully halted disease progression in one patient, transplantation to a second patient was discontinued due to genomic aberrance in the iPSC. Therefore, identifying the mechanisms that lead to genomic instability in A-iPSC and developing methods to correct them are imperative for clinical use of iPSC-based therapies in older patients and patients characterized by an aged phenotype, which may result from lifestyle (e.g., smoking, excessive alcohol intake) and/or the aging process.

### SUMMARY

In one aspect, the present disclosure provides a method of producing induced pluripotent stem cells (iPSCs) from mammalian non-pluripotent cells, wherein the iPSCs are characterized by one or more of increased genomic stability, increased DNA damage response, increased ZSCAN10 expression, and reduced glutathione synthetase (GSS) expression, the method comprising: culturing non-pluripotent cells treated with an effective amount of glutathione or derivatives thereof prior to the initiation of reprogramming, during reprogramming, and/or after reprogramming of the non-pluripotent cells under conditions that allow for the production of iPSCs, thereby producing iPSCs with one or more of increased genomic stability, increased DNA damage response, increased ZSCAN10 expression, and reduced GSS expression as compared to iPSCs produced from untreated control non-pluripotent cells grown under similar conditions.

In some embodiments, the method further comprises identifying non-pluripotent cells for treatment with glutathione or derivatives thereof, wherein the non-pluripotent cells identified for treatment express an elevated cellular reactive oxygen species (ROS) level prior to treatment relative to that observed in untreated control non-pluripotent cells, wherein the elevated cellular ROS level identifies the non-pluripotent cells for treatment with glutathione or derivatives thereof and the lack of the elevated cellular ROS level does not identify the non-pluripotent cells for treatment with glutathione or derivatives thereof.

In some embodiments, the efficiency of reprogramming the non-pluripotent cells treated with glutathione or derivatives thereof is increased relative to untreated control non-pluripotent cells.

In some embodiments, treatment with the glutathione or derivatives thereof increases the efficiency of reprogramming the non-pluripotent cells into iPSCs by at least 10-fold relative to untreated control non-pluripotent cells.

In some embodiments, treatment with glutathione or derivatives thereof restores ZSCAN10 expression levels in iPSCs to about 50% or more of the respective expression levels of embryonic stem cells (ESCs).

In some embodiments, the mammalian non-pluripotent cells are somatic cells. In some embodiments, the somatic cells are aged somatic cells. In some embodiments, the somatic cells are somatic cells from an embryonic stage.

In some embodiments, the somatic cells express an increased cellular ROS level relative to that observed in young somatic cells. In some embodiments, the somatic cells are incapable of generating iPSCs.

In some embodiments, the somatic cells are selected from the group consisting of: fibroblast cells, cells from blood, cells from ocular tissue, epithelial cells, osteocytes, chondrocytes, neurons, muscle cells, hepatic cells, intestinal cells, spleen cells, adult stem cells, and progenitor cells from adult stem cells. In some embodiments, the mammalian non-pluripotent cells are progenitor cells.

In one aspect, the present disclosure provides induced pluripotent stem cells (iPSCs) produced by a method of producing iPSCs from mammalian non-pluripotent cells, wherein the iPSCs are characterized by one or more of increased genomic stability, increased DNA damage response, increased ZSCAN10 expression, and reduced glutathione synthetase (GSS) expression, the method comprising: culturing non-pluripotent cells treated with an effective amount of glutathione or derivatives thereof prior to the initiation of reprogramming, during reprogramming, and/or after reprogramming of the non-pluripotent cells under conditions that allow for the production of iPSCs, wherein the iPSCs produced from the non-pluripotent cells treated with glutathione or derivatives thereof are characterized by one or more of increased genomic stability, increased DNA damage response, increased iPSC reprogramming efficiency, increased ZSCAN10 expression, and reduced GSS expression as compared to iPSCs produced from untreated control non-pluripotent cells grown under similar conditions.

In some embodiments, the iPSCs are characterized by increased genomic stability as compared to iPSCs produced from untreated control non-pluripotent cells grown under similar conditions.

In some embodiments, the iPSCs are characterized by increased DNA damage response as compared to iPSCs produced from untreated control non-pluripotent cells grown under similar conditions.

In some embodiments, the iPSCs are characterized by increased ZSCAN10 expression as compared to iPSCs produced from untreated control non-pluripotent cells grown under similar conditions.

In some embodiments, the iPSCs are characterized by reduced GSS expression as compared to iPSCs produced from untreated control non-pluripotent cells grown under similar conditions.

In some embodiments, the iPSCs are characterized by increased iPSC reprogramming efficiency as compared to iPSCs produced from untreated control non-pluripotent cells grown under similar conditions.

In some embodiments, the glutathione is glutathione reduced ethyl ester.

In one aspect, the present disclosure provides a method of producing induced pluripotent stem cells derived from aged somatic cells (A-iPSCs) having one or more of increased genomic stability, increased DNA damage response, increased ZSCAN10 expression, and reduced glutathione synthetase (GSS) expression, the method comprising: culturing aged somatic cells treated with an effective amount of glutathione or derivatives thereof prior to the initiation of reprogramming, during reprogramming, and/or after reprogramming of the aged somatic cells under conditions that allow for the production of A-iPSCs, thereby producing A-iPSCs with one or more of increased genomic stability, increased DNA damage response, increased iPSC reprogramming efficiency, increased ZSCAN10 expression, and reduced GSS expression as compared to that observed in A-iPSCs produced from untreated control aged somatic cells grown under similar conditions and/or comparable to that observed in young iPSCs (Y-iPSCs) or ESCs.

In some embodiments, the method further comprises identifying aged somatic cells for treatment with glutathione or derivatives thereof, wherein the aged somatic cells identified for treatment express an elevated cellular reactive oxygen species (ROS) level prior to treatment relative to one or more of untreated control aged somatic cells, young somatic cells, and ESCs, wherein the elevated cellular ROS level identifies the aged somatic cells for treatment with glutathione or derivatives thereof and the lack of the elevated cellular ROS level does not identify the aged somatic cells for treatment with glutathione or derivatives thereof.

In one aspect, the present disclosure provides A-iPSCs produced by a method of producing A-iPSCs having one or more of increased genomic stability, increased DNA damage response, increased iPSC reprogramming efficiency, increased ZSCAN10 expression, and reduced glutathione synthetase (GSS) expression, the method comprising: culturing aged somatic cells treated with an effective amount of glutathione or derivatives thereof prior to the initiation of reprogramming, during reprogramming, and/or after reprogramming of the aged somatic cells under conditions that allow for the production of A-iPSCs, wherein the A-iPSCs produced from the aged somatic cells treated with glutathione or derivatives thereof are characterized by one or more of increased genomic stability, increased DNA damage response, increased iPSC reprogramming efficiency, increased ZSCAN10 expression, and reduced GSS expression as compared to that observed in A-iPSCs produced from untreated control aged somatic cells grown under similar conditions and/or comparable to that observed in young iPSCs (Y-iPSCs) or ESCs.

In some embodiments, the A-iPSCs are characterized by increased genomic stability as compared to A-iPSCs produced from untreated control aged somatic cells grown under similar conditions and/or comparable to that observed in young iPSCs (Y-iPSCs) or ESCs.

In some embodiments, the A-iPSCs are characterized by increased DNA damage response as compared to A-iPSCs produced from untreated control aged somatic cells grown under similar conditions and/or comparable to that observed in young iPSCs (Y-iPSCs) or ESCs.

In some embodiments, the A-iPSCs are characterized by increased iPSC reprogramming efficiency as compared to A-iPSCs produced from untreated control aged somatic cells grown under similar conditions and/or comparable to that observed in young iPSCs (Y-iPSCs) or ESCs.

In some embodiments, the A-iPSCs are characterized by increased ZSCAN10 expression as compared to A-iPSCs produced from untreated control aged somatic cells grown under similar conditions and/or comparable to that observed in young iPSCs (Y-iPSCs) or ESCs.

In some embodiments, the A-iPSCs are characterized by reduced glutathione synthetase (GSS) expression as compared to A-iPSCs produced from untreated control aged somatic cells grown under similar conditions and/or comparable to that observed in young iPSCs (Y-iPSCs) or ESCs.

In some embodiments, the glutathione is glutathione reduced ethyl ester.

In one aspect, the present disclosure provides a method of producing pluripotent stem cells including embryonic stem cell derivation from blastocyst, parthenogenetic ES cells, nuclear transferred ES cells to improve genomic stability, derivation efficiency, and reprogramming quality comprising: culturing embryos treated with an effective amount of glutathione or derivatives thereof prior to the initiation of reprogramming and/or during reprogramming of the embryos under conditions that allow for the production of ES cells, parthenogenetic ES cells, nuclear transferred ES cells to minimize the oxidative stress (ROS)-mediated inhibitory effects during reprogramming of the pluripotent stem cells, thereby producing pluripotent stem cells with one or more of improved genomic stability, improved DNA damage response, reprogramming quality with increased pluripotent gene expression including ZSCAN10 expression and reduced GSS expression as compared to the pluripotent stem cells produced from untreated control cells grown under similar conditions.

In one aspect, the present disclosure provides a method for stem cell therapy comprising: (a) isolating a non-pluripotent cell from a subject; (b) producing an iPSC by a method of producing iPSCs from mammalian non-pluripotent cells, wherein the iPSCs are characterized by one or more of increased genomic stability, increased DNA damage response, increased ZSCAN10 expression, and reduced glutathione synthetase (GSS) expression, the method comprising: culturing non-pluripotent cells treated with an effective amount of glutathione or derivatives thereof prior to the initiation of reprogramming, during reprogramming, and/or after reprogramming of the non-pluripotent cells under conditions that allow for the production of iPSCs, wherein the iPSCs produced from the non-pluripotent cells treated with glutathione or derivatives thereof are characterized by one or more of increased genomic stability, increased DNA damage response, increased iPSC reprogramming efficiency, increased ZSCAN10 expression, and reduced GSS expression as compared to iPSCs produced from untreated control non-pluripotent cells grown under similar conditions; (c) differentiating the iPSC ex vivo into a differentiated cell; and (d) administering the differentiated cell to the subject.

In one aspect, the present disclosure provides a method for stem cell therapy comprising: (a) isolating an aged somatic cell from a subject; (b) producing an A-iPSC by a method of producing A-iPSCs having one or more of increased genomic stability, increased DNA damage response, increased iPSC reprogramming efficiency, increased ZSCAN10 expression, and reduced glutathione synthetase (GSS) expression, the method comprising: culturing aged somatic cells treated with an effective amount of glutathione or derivatives thereof prior to the initiation of reprogramming, during reprogramming, and/or after reprogramming of the aged somatic cells under conditions that allow for the production of A-iPSCs, wherein the A-iPSCs produced from the aged somatic cells treated with glutathione or derivatives thereof are characterized by one or more of increased genomic stability, increased DNA damage response, increased iPSC reprogramming efficiency, increased ZSCAN10 expression, and reduced GSS expression as compared to that observed in A-iPSCs produced from untreated control aged somatic cells grown under similar conditions and/or comparable to that observed in young iPSCs (Y-iPSCs) or ESCs; (c) differentiating the A-iPSC ex vivo into a differentiated cell; and (d) administering the differentiated cell to the subject.

In some embodiments, the elevated cellular ROS level of the non-pluripotent cells identified for treatment is defined by a metabolic profile comprising one or more metabolites exhibiting increased levels relative to that observed in untreated control non-pluripotent cells.

In some embodiments, the one or more metabolites exhibiting increased levels is selected from the group consisting of adenosine, cytidine, xanthine, and cytidine 3′ monophosphate (3′-CMP).

In some embodiments, the elevated cellular ROS level of the non-pluripotent cells identified for treatment is defined by an increased gene expression level of one or more genes selected from the group consisting of ST6GALNAC6, IGFBP5, PDGFD, SURF4, BOC, ADGRD1, MPDU1, RPS4Y1, MME, SET, DOK1, COLEC12, HOXC10, SULF2, ADAMTSL1, ELN, MGRN1, COL15A1, ZEB1, SFRP1, CLDN11, LGALS3BP, CHI3L1, SPG21, PI16, and MCFD2 relative to that observed in untreated control non-pluripotent cells.

In some embodiments, the gene expression level of the one or more genes in the non-pluripotent cells identified for treatment is increased by about 2-fold to about 5-fold relative to that observed in untreated control non-pluripotent cells.

In some embodiments, the gene expression level of the one or more genes in the non-pluripotent cells identified for treatment is increased by about 5-fold relative to that observed in untreated control non-pluripotent cells.

In some embodiments, the elevated cellular ROS level of the non-pluripotent cells identified for treatment is defined by an increased cellular G-quadruplex (G4) DNA structure formation relative to that observed in untreated control non-pluripotent cells.

In some embodiments, the G4 DNA structure formation in the non-pluripotent cells identified for treatment is increased by about 2-fold relative to that observed in untreated control non-pluripotent cells.

In some embodiments, the elevated cellular ROS level of the non-pluripotent cells identified for treatment is defined by an increased 8-oxo-guanine (oxoG) formation relative to that observed in untreated control non-pluripotent cells.

In some embodiments, the oxoG formation in the non-pluripotent cells identified for treatment is increased by about 2-fold to about 3-fold relative to that observed in untreated control non-pluripotent cells.

In some embodiments, the elevated cellular ROS level of the aged somatic cells identified for treatment is defined by a metabolic profile comprising one or more metabolites exhibiting increased levels relative to that observed in one or more of untreated control aged somatic cells, young somatic cells, and ESCs.

In some embodiments, the one or more metabolites exhibiting increased levels is selected from the group consisting of adenosine, cytidine, xanthine, and cytidine 3′ monophosphate (3′-CMP).

In some embodiments, the elevated cellular ROS level of the aged somatic cells identified for treatment is defined by an increased gene expression level of one or more genes selected from the group consisting of ST6GALNAC6, IGFBP5, PDGFD, SURF4, BOC, ADGRD1, MPDU1, RPS4Y1, MME, SET, DOK1, COLEC12, HOXC10, SULF2, ADAMTSL1, ELN, MGRN1, COL15A1, ZEB1, SFRP1, CLDN11, LGALS3BP, CHI3L1, SPG21, PI16, and MCFD2 relative to that observed in one or more of untreated control aged somatic cells, young somatic cells, and ESCs.

In some embodiments, the gene expression level of the one or more genes in the aged somatic cells identified for treatment is increased by about 2-fold to about 5-fold relative to that observed in one or more of untreated control aged somatic cells, young somatic cells, and ESCs.

In some embodiments, the gene expression level of the one or more genes in the aged somatic cells identified for treatment is increased by about 5-fold relative to that observed in one or more of untreated control aged somatic cells, young somatic cells, and ESCs.

In some embodiments, the elevated cellular ROS level of the aged somatic cells identified for treatment is defined by an increased cellular G-quadruplex (G4) DNA structure formation relative to that observed in one or more of untreated control aged somatic cells, young somatic cells, and ESCs.

In some embodiments, the G4 DNA structure formation in the aged somatic cells identified for treatment is increased by about 2-fold relative to that observed in one or more of untreated control aged somatic cells, young somatic cells, and ESCs.

In some embodiments, the elevated cellular ROS level of the aged somatic cells identified for treatment is defined by an increased 8-oxo-guanine (oxoG) formation relative to that observed in one or more of untreated control aged somatic cells, young somatic cells, and ESCs.

In some embodiments, the oxoG formation in the aged somatic cells identified for treatment is increased by about 2-fold to about 3-fold relative to that observed in one or more of untreated control aged somatic cells, young somatic cells, and ESCs.

In some embodiments, the method further comprises identifying embryonic stem cell derivation from blastocyst, parthenogenetic ES cells, or nuclear transferred ES cells for treatment with glutathione or derivatives thereof, wherein the embryonic stem cell derivation from blastocyst, parthenogenetic ES cells, or nuclear transferred ES cells identified for treatment express an elevated reactive oxygen species (ROS) level prior to treatment relative to one or more of untreated control embryonic stem cell derivation from blastocyst, parthenogenetic ES cells, or nuclear transferred ES cells, wherein the elevated cellular ROS level identifies the embryonic stem cell derivation from blastocyst, parthenogenetic ES cells, or nuclear transferred ES cells for treatment with glutathione or derivatives thereof and the lack of the elevated cellular ROS level does not identify the embryonic stem cell derivation from blastocyst, parthenogenetic ES cells, or nuclear transferred ES cells for treatment with glutathione or derivatives thereof.

In some embodiments, the elevated cellular ROS level of the embryonic stem cell derivation from blastocyst, parthenogenetic ES cells, or nuclear transferred ES cells identified for treatment is defined by a metabolic profile comprising one or more metabolites exhibiting increased levels relative to that observed in one or more of untreated control embryonic stem cell derivation from blastocyst, parthenogenetic ES cells, or nuclear transferred ES cells.

In some embodiments, the one or more metabolites exhibiting increased levels is selected from the group consisting of adenosine, cytidine, xanthine, and cytidine 3′ monophosphate (3′-CMP).

In some embodiments, the elevated cellular ROS level of the embryonic stem cell derivation from blastocyst, parthenogenetic ES cells, or nuclear transferred ES cells identified for treatment is defined by an increased gene expression level of one or more genes selected from the group consisting of ST6GALNAC6, IGFBP5, PDGFD, SURF4, BOC, ADGRD1, MPDU1, RPS4Y1, MME, SET, DOK1, COLEC12, HOXC10, SULF2, ADAMTSL1, ELN, MGRN1, COL15A1, ZEB1, SFRP1, CLDN11, LGALS3BP, CHI3L1, SPG21, PI16, and MCFD2 relative to that observed in one or more of untreated control embryonic stem cell derivation from blastocyst, parthenogenetic ES cells, or nuclear transferred ES cells.

In some embodiments, the gene expression level of the one or more genes in the embryonic stem cell derivation from blastocyst, parthenogenetic ES cells, or nuclear transferred ES cells identified for treatment is increased by about 2-fold to about 5-fold relative to that observed in one or more of untreated control embryonic stem cell derivation from blastocyst, parthenogenetic ES cells, or nuclear transferred ES cells.

In some embodiments, the gene expression level of the one or more genes in the embryonic stem cell derivation from blastocyst, parthenogenetic ES cells, or nuclear transferred ES cells identified for treatment is increased by about 5-fold relative to that observed in one or more of untreated control embryonic stem cell derivation from blastocyst, parthenogenetic ES cells, or nuclear transferred ES cells.

In some embodiments, the elevated cellular ROS level of the embryonic stem cell derivation from blastocyst, parthenogenetic ES cells, or nuclear transferred ES cells identified for treatment is defined by an increased cellular G-quadruplex (G4) DNA structure formation relative to that observed in one or more of untreated control embryonic stem cell derivation from blastocyst, parthenogenetic ES cells, or nuclear transferred ES cells.

In some embodiments, the G4 DNA structure formation in the embryonic stem cell derivation from blastocyst, parthenogenetic ES cells, or nuclear transferred ES cells identified for treatment is increased by about 2-fold relative to that observed in one or more of untreated control embryonic stem cell derivation from blastocyst, parthenogenetic ES cells, or nuclear transferred ES cells.

In some embodiments, the elevated ROS level of the embryonic stem cell derivation from blastocyst, parthenogenetic ES cells, or nuclear transferred ES cells identified for treatment is defined by an increased 8-oxo-guanine (oxoG) formation relative to that observed in one or more of untreated control embryonic stem cell derivation from blastocyst, parthenogenetic ES cells, or nuclear transferred ES cells.

In some embodiments, the oxoG formation in the embryonic stem cell derivation from blastocyst, parthenogenetic ES cells, or nuclear transferred ES cells identified for treatment is increased by about 2-fold to about 3-fold relative to that observed in one or more of untreated control embryonic stem cell derivation from blastocyst, parthenogenetic ES cells, or nuclear transferred ES cells.

In one aspect, the present disclosure provides a method for stem cell therapy comprising: (a) isolating a non-pluripotent cell from a subject; (b) producing an iPSC by a method of producing iPSCs from mammalian non-pluripotent cells, wherein the iPSCs are characterized by one or more of increased genomic stability, increased DNA damage response, increased ZSCAN10 expression, and reduced glutathione synthetase (GSS) expression, the method comprising: culturing non-pluripotent cells treated with an effective amount of glutathione or derivatives thereof prior to the initiation of reprogramming, during reprogramming, and/or after reprogramming of the non-pluripotent cells under conditions that allow for the production of iPSCs, wherein the iPSCs produced from the non-pluripotent cells treated with glutathione or derivatives thereof are characterized by one or more of increased genomic stability, increased DNA damage response, increased iPSC reprogramming efficiency, increased ZSCAN10 expression, and reduced GSS expression as compared to iPSCs produced from untreated control non-pluripotent cells grown under similar conditions, wherein the non-pluripotent cells identified for treatment express an elevated cellular reactive oxygen species (ROS) level prior to treatment relative to that observed in untreated control non-pluripotent cells, wherein the elevated cellular ROS level identifies the non-pluripotent cells for treatment with glutathione or derivatives thereof and the lack of the elevated cellular ROS level does not identify the non-pluripotent cells for treatment with glutathione or derivatives thereof; (c) differentiating the iPSC ex vivo into a differentiated cell; and (d) administering the differentiated cell to the subject.

In some embodiments, the elevated cellular ROS level of the non-pluripotent cells identified for treatment is defined by a metabolic profile comprising one or more metabolites exhibiting increased levels relative to that observed in untreated control non-pluripotent cells.

In some embodiments, the one or more metabolites exhibiting increased levels is selected from the group consisting of adenosine, cytidine, xanthine, and cytidine 3′ monophosphate (3′-CMP).

In some embodiments, the elevated cellular ROS level of the non-pluripotent cells identified for treatment is defined by an increased gene expression level of one or more genes selected from the group consisting of ST6GALNAC6, IGFBP5, PDGFD, SURF4, BOC, ADGRD1, MPDU1, RPS4Y1, MME, SET, DOK1, COLEC12, HOXC10, SULF2, ADAMTSL1, ELN, MGRN1, COL15A1, ZEB1, SFRP1, CLDN11, LGALS3BP, CHI3L1, SPG21, PI16, and MCFD2 relative to that observed in untreated control non-pluripotent cells.

In some embodiments, the gene expression level of the one or more genes in the non-pluripotent cells identified for treatment is increased by about 2-fold to about 5-fold relative to that observed in untreated control non-pluripotent cells.

In some embodiments, the gene expression level of the one or more genes in the non-pluripotent cells identified for treatment is increased by about 5-fold relative to that observed in untreated control non-pluripotent cells.

In some embodiments, the elevated cellular ROS level of the non-pluripotent cells identified for treatment is defined by an increased cellular G-quadruplex (G4) DNA structure formation relative to that observed in untreated control non-pluripotent cells.

In some embodiments, the G4 DNA structure formation in the non-pluripotent cells identified for treatment is increased by about 2-fold relative to that observed in untreated control non-pluripotent cells.

In some embodiments, the elevated cellular ROS level of the non-pluripotent cells identified for treatment is defined by an increased 8-oxo-guanine (oxoG) formation relative to that observed in untreated control non-pluripotent cells.

In some embodiments, the oxoG formation in the non-pluripotent cells identified for treatment is increased by about 2-fold to about 3-fold relative to that observed in untreated control non-pluripotent cells.

In one aspect, the present disclosure provides a method for stem cell therapy comprising: (a) isolating an aged somatic cell from a subject; (b) producing an A-iPSC by a method of producing A-iPSCs having one or more of increased genomic stability, increased DNA damage response, increased iPSC reprogramming efficiency, increased ZSCAN10 expression, and reduced glutathione synthetase (GSS) expression, the method comprising: culturing aged somatic cells treated with an effective amount of glutathione or derivatives thereof prior to the initiation of reprogramming, during reprogramming, and/or after reprogramming of the aged somatic cells under conditions that allow for the production of A-iPSCs, wherein the A-iPSCs produced from the aged somatic cells treated with glutathione or derivatives thereof are characterized by one or more of increased genomic stability, increased DNA damage response, increased iPSC reprogramming efficiency, increased ZSCAN10 expression, and reduced GSS expression as compared to that observed in A-iPSCs produced from untreated control aged somatic cells grown under similar conditions and/or comparable to that observed in young iPSCs (Y-iPSCs) or ESCs, wherein the aged somatic cells identified for treatment express an elevated cellular reactive oxygen species (ROS) level prior to treatment relative to one or more of untreated control aged somatic cells, young somatic cells, and ESCs, wherein the elevated cellular ROS level identifies the aged somatic cells for treatment with glutathione or derivatives thereof and the lack of the elevated cellular ROS level does not identify the aged somatic cells for treatment with glutathione or derivatives thereof; (c) differentiating the A-iPSC ex vivo into a differentiated cell; and (d) administering the differentiated cell to the subject.

In some embodiments, the elevated cellular ROS level of the aged somatic cells identified for treatment is defined by a metabolic profile comprising one or more metabolites exhibiting increased levels relative to that observed in one or more of untreated control aged somatic cells, young somatic cells, and ESCs.

In some embodiments, the one or more metabolites exhibiting increased levels is selected from the group consisting of adenosine, cytidine, xanthine, and cytidine 3′ monophosphate (3′-CMP).

In some embodiments, the elevated cellular ROS level of the aged somatic cells identified for treatment is defined by an increased gene expression level of one or more genes selected from the group consisting of ST6GALNAC6, IGFBP5, PDGFD, SURF4, BOC, ADGRD1, MPDU1, RPS4Y1, MME, SET, DOK1, COLEC12, HOXC10, SULF2, ADAMTSL1, ELN, MGRN1, COL15A1, ZEB1, SFRP1, CLDN11, LGALS3BP, CHI3L1, SPG21, PI16, and MCFD2 relative to that observed in one or more of untreated control aged somatic cells, young somatic cells, and ESCs.

In some embodiments, the gene expression level of the one or more genes in the aged somatic cells identified for treatment is increased by about 2-fold to about 5-fold relative to that observed in one or more of untreated control aged somatic cells, young somatic cells, and ESCs.

In some embodiments, the gene expression level of the one or more genes in the aged somatic cells identified for treatment is increased by about 5-fold relative to that observed in one or more of untreated control aged somatic cells, young somatic cells, and ESCs.

In some embodiments, the elevated cellular ROS level of the aged somatic cells identified for treatment is defined by an increased cellular G-quadruplex (G4) DNA structure formation relative to that observed in one or more of untreated control aged somatic cells, young somatic cells, and ESCs.

In some embodiments, the G4 DNA structure formation in the aged somatic cells identified for treatment is increased by about 2-fold relative to that observed in one or more of untreated control aged somatic cells, young somatic cells, and ESCs.

In some embodiments, the elevated cellular ROS level of the aged somatic cells identified for treatment is defined by an increased 8-oxo-guanine (oxoG) formation relative to that observed in one or more of untreated control aged somatic cells, young somatic cells, and ESCs.

In some embodiments, the oxoG formation in the aged somatic cells identified for treatment is increased by about 2-fold to about 3-fold relative to that observed in one or more of untreated control aged somatic cells, young somatic cells, and ESCs.

In one aspect, the present disclosure provides a kit comprising glutathione reduced ethyl ester, reprogramming factors, and instructions for reprogramming a plurality of non-pluripotent cells.

### BRIEF DESCRIPTION OF THE DRAWINGS

FIG. 1. Impaired genomic integrity and DNA damage response of mouse A-iPSC compared to Y-iPSC and ESC, and recovery following transient expression of ZSCAN10. (A) Number of chromosomal abnormalities observed by cytogenetic analysis in each A-iPSC clone, and recovery with ZSCAN10 expression. Error bars indicate standard error of the mean of independent clones analyzed per group. The total number of metaphases analyzed is indicated in each group. Statistical significance was determined by two-sided t-test. (B) In situ cell death assays of ESC, Y-iPSC, A-iPSC, and A-iPSC-ZSCAN10 were performed 15 hours after the end of phleomycin treatment (2 h, 30 μg/ml). A-iPSC show fewer cells staining for cell death compared to ESC, Y-iPSC, and A-iPSC-ZSCAN10. The negative control is Y-iPSC treated with dye in the absence of enzymatic reaction. Nuclei are stained with DAPI. Scale bar indicates 100 μm. (C) Quantification by image-analysis of apoptotic response by DNA fragmentation assay after phleomycin treatment. Error bars indicate standard error of the mean of technical and biological replicates. The exact number of biological replicates is indicated below each group. Statistical significance was determined by unpaired two-sided t-test. (D) Reduced ATM phosphorylation (pATM) in A-iPSC as monitored by immunoblot after phleomycin treatment (2 h, 30 μg/ml), and recovery of ATM activation upon ZSCAN10 expression. The red line indicates the same ESC sample loaded in both immunoblots as an internal control. (E) Immunohistochemistry showing low γ-H2AX (phosphorylated H2AX) in A-iPSC after phleomycin treatment (2 h, 30 μg/ml) and recovery of γ-H2AX signal with ZSCAN10 expression. (F) Ratio of γ-H2AX-positive cells to DAPI-stained nuclei quantified by immunostaining. Only cells showing punctate γ-H2AX foci were counted. The number of independent colonies counted is indicated in each group. Error bars indicate standard error of the mean of independent colonies. Statistical significance was determined by unpaired two-sided t-test. (G) Immunoblot analysis of γ-H2AX confirms the immunohistochemistry findings. (H) Immunoblot showing impaired p53 DNA damage response in A-iPSC and recovery with transient expression of ZSCAN10 in three independent clones after phleomycin treatment (2 h, 30 μg/ml). Red line indicates the same ESC sample loaded in both immunoblots as an internal control.

FIG. 2. Evaluation of ZSCAN10 function on DNA damage response and genomic stability of mouse A-iPSC compared to Y-iPSC and ESC. (A) pATM immunoblot illustrating the differential DNA damage response of A-ntESC and A-iPSC generated from an aged tissue donor. Three independent clones of A-ntESC show a normal DNA damage response after phleomycin treatment. (B) Q-PCR of ZSCAN10 mRNA levels showing poor activation of ZSCAN10 expression in A-iPSC and complete activation with transient expression of ZSCAN10. Endogenous ZSCAN10 levels normalized to β-actin. Error bars indicate standard error of the mean of two technical replicates with three independent clones in each sample group. Statistical significance was determined by two-sided t-test. (C) Immunoblot showing impaired ATM/H2AX/p53 DNA damage response in Y-iPSC with ZSCAN10 shRNA expression in three independent clones after phleomycin treatment (2 h, 30 μg/ml). (D) ATM/H2AX/p53-mediated DNA damage response after irradiation. ESC and Y-iPSC, but not A-iPSC, show an increase in pATM/γ-H2AX/p53 level after irradiation. The ATM/H2AX/p53 response to irradiation in A-iPSC is recovered by transient expression of ZSCAN10. (E) Estimation of higher mutation rate in A-iPSC, and recovery with ZSCAN10 expression. The mutation frequency was estimated by the inactivation of HPRT promoter activity in the presence of 6-thioguanine-mediated negative selection, and confirmed by Q-PCR. Error bars indicate standard error of the mean of three biological replicates with four independent clones in each sample group. Statistical significance was determined by two-sided t-test.

FIG. 3. Imbalance of ROS-glutathione homeostasis in mouse A-iPSC, and recovery by ZSCAN10 expression via reduction of excessively activated GSS. (A-B) ZSCAN10 expression in A-iPSC influences the expression of pluripotency genes, making the A-iPSC gene expression profile more similar to that of Y-iPSC. Whole-genome expression profiles of aged and young fibroblast cells (A-SC, Y-SC), ESC, Y-iPSC, A-iPSC, and A-iPSC-ZSCAN10 with independent clones for each somatic cells and pluripotent cells as biological repeats (n≥2) were included in the analysis (A). Principal Component Analysis (PCA) using whole-genome expression profiles (B). Heatmap shows the hierarchical clustering of samples and pairwise gene expression similarities measured by Pearson correlation coefficient. (C) Q-PCR of GSS mRNA levels indicating excessive expression in A-iPSC and downregulation with ZSCAN10 expression. Error bars indicate standard error of the mean of two technical replicates with three independent clones in each sample group. Statistical significance was determined by two-sided t-test. (D) Excessive oxidation capacity with elevated glutathione in A-iPSC, and recovery by ZSCAN10 expression. The total glutathione level was measured to determine the maximum oxidation capacity. Excessive oxidation capacity of glutathione in A-iPSC is normalized to the level of ESC and Y-iPSC by transient expression of ZSCAN10. Mean±standard deviation is plotted for four biological replicates with two independent clones in each sample group from each condition. Glutathione analysis was conducted with the Glutathione Fluorometric Assay. Statistical significance was determined by two-sided t-test. (E) ROS scavenging activity of ESC, ntESC, Y-iPSC, A-iPSC-ZSCAN10, and A-iPSC. A cellular ROS assay kit (DCFDA assay) was used to measure H₂O₂ scavenging activity. A-iPSC show strong H₂O₂ scavenging activity, with a reduced response against treatment with TBHP (tert-butyl hydrogen peroxide; stable chemical form of H₂O₂, 3 h); the response is recovered by ZSCAN10 expression. Mean±standard deviation is plotted for three biological replicates with two independent clones in each sample group from each condition. Statistical significance was determined by two-sided t-test. (F) Apoptosis assay in A-iPSC with GSS shRNA expression and Y-iPSC-GSS by image quantification. A lower apoptotic response (DNA fragmentation assay) is seen 15 h after the end of phleomycin treatment (2 hours, 30 μg/ml) in A-iPSC, and is recovered with GSS downregulation in A-iPSC. Transient expression of GSS in Y-iPSC also reduces apoptotic response. Error bars indicate standard error of the mean of three biological replicates with two independent clones in each sample group. Statistical significance was determined by two-sided t-test. (G) Immunoblot of pATM showing recovery of the DNA damage response after phleomycin treatment in three independent clones of A-iPSC with shRNA-mediated knockdown of GSS. (H) Immunoblot of pATM/γ-H2AX/p53 showing that lentiviral expression of GSS cDNA impairs DNA damage response in three independent clones of Y-iPSC after phleomycin treatment.

FIG. 4. Evaluation of ZSCAN10 function on DNA damage response and genomic integrity in human A-hiPSC. (A) Immunoblots showing the levels of pATM and β-actin proteins with three imported A-hiPSC clones with known abnormal cytogenetic signature. (B) Q-PCR of ZSCAN10. Error bars indicate standard error of the mean of two technical replicates with three independent clones in each sample group except two technical and three biological replicates in the sample of A-iPSC-JA and A-iPSC-LS. Statistical significance was determined by two-sided t-test. (C) Q-PCR of GSS. Error bars indicate standard error of the mean of two technical replicates with three independent clones in each sample group except two technical and three biological replicates in the sample of A-iPSC-JA and A-iPSC-LS. Statistical significance was determined by two-sided t-test. (D) Immunoblots showing the levels of pATM and β-actin in five independent clones of A-hiPSC, five independent clones of Y-hiPSC, and five clones of A-hiPSC expressing ZSCAN10. (E) Immunoblot showing impaired ATM DNA damage response in Y-hiPSC with ZSCAN10 shRNA expression in three independent clones after phleomycin treatment (2 h, 30 μg/ml). (F) Copy number profiling analysis of Y-hiPSC with ZSCAN10 shRNA expression in four independent clones.

FIG. 5. Impaired DNA damage response in human A-hiPSC caused by deregulation of ZSCAN10 and GSS and recovered by ZSCAN10 expression. (A) Excessive oxidation capacity with elevated glutathione in A-hiPSC, and recovery by ZSCAN10 expression. The total glutathione level was measured to determine the maximum oxidation capacity. Excessive oxidation capacity of glutathione in A-hiPSC is normalized to the level of hESC and Y-hiPSC by transient expression of ZSCAN10. Glutathione analysis was conducted with the Glutathione Fluorometric Assay. Mean±standard deviation is plotted for three biological replicates with two independent clones in each sample group from each condition. Statistical significance was determined by two-sided t-test. (B) ROS scavenging activity of hESC, Y-hiPSC, A-hiPSC, and A-hiPSC-ZSCAN10. A cellular ROS assay kit (DCFDA assay) was used to measure H₂O₂ scavenging activity. A-hiPSC show strong H₂O₂ scavenging activity, with a reduced response against treatment with TBHP (tert-butyl hydrogen peroxide; stable chemical form of H₂O₂, 3 h); the response is recovered by ZSCAN10 expression. Mean±standard deviation is plotted for four biological replicates in each sample group from each condition. Statistical significance was determined by two-sided t-test. (C) Immunoblot of pATM showing recovery of the DNA damage response after phleomycin treatment in three independent clones of A-hiPSC with shRNA-mediated knockdown of GSS. (D) Immunoblot of pATM showing that lentiviral expression of GSS cDNA impairs the DNA damage response in three independent clones of Y-hiPSC after phleomycin treatment. (E-G) Copy number profiling analysis of human iPSC. Schematic diagrams represent seven rearranged A-hiPSC, four non-rearranged A-hiPSC, and five non-rearranged A-hiPSC-ZSCAN10 in the genetically controlled setting of A-hiPSC. Ten non-rearranged Y-hiPSC, which were generated from different tissue donors, were also included. A-hiPSC (n=11 (7/11), p=0.64, A-hiPSC (n=5 (0/5), p*=6.3E-3) and Y-hiPSC (n=10 (0/10), p*<4E-5). The number in parenthesis represents detected rearrangements and p and p* are the observed and estimated likelihoods of detecting no rearrangements in the absence of lineage effects using a binomial distribution, respectively.

FIG. 6. Somatic cell ROS as a causative origin of the genomic instability in A-iPSC and recovery by glutathione treatment in the early stage of A-iPSC reprogramming. (A) Somatic cell ROS measured by MitoSOX staining. Mitochondrial Superoxide Indicator, MitoSOX Red dye (ThermoFisher, M36008) was used to measure somatic cell ROS. (B) Quantification of the MitoSOX staining level using image-based quantification (ImageJ software). Error bars indicate standard error of the mean of independent colonies. Statistical significance was determined by unpaired two-sided t-test. (C) Schematic demonstration of illustrative A-iPSC reprogramming with the stabilized form of glutathione (3 mM of glutathione reduced ethyl ester, CAT #G-275-500, GoldBio) from one day before reprogramming virus infection to 10 days post reprogramming virus infection. (D) Copy number profiling analysis of human A-iPSC with glutathione treatment. Schematic diagrams represent 10 non-rearranged A-iPSC with glutathione treatment, compared with seven rearranged A-iPSC from 11 clones without glutathione treatment. (E) Immunoblot of pATM showing that A-iPSC with glutathione treatment impairs the DNA damage response in the biologically independent clones after phleomycin treatment.

FIG. 7. Higher somatic cell ROS among the tissue donors as a causative origin of the genomic instability in A-iPSC and recovery by glutathione treatment in the early stage of A-iPSC reprogramming. (A-B) Somatic cell ROS measured by MitoSOX staining. Mitochondrial Superoxide Indicator, MitoSOX Red dye (ThermoFisher, M36008) was used to measure somatic cell ROS from young somatic cells (Y-SC) from B6CBA mouse and aged somatic cells (A-SC) from B6129 and B6CBA mice (A), and human young somatic cells (Y-SC) from MRCS donor and human aged somatic cells (A-SC) from LS and AG4 donors (B). Reduced level of the somatic cell ROS with the treatment of the stabilized form of glutathione chemical (3 mM of glutathione reduced ethyl ester, CAT #G-275-500, GoldBio) for three days in the media in A-SC from AG4 donor (A-SC-AG4-glutathione) (B). Scale bar indicates 100 (C-D) Quantification of the MitoSOX staining (ROS) level using image-based quantification (ImageJ software) from the samples in FIGS. 7A and 7B. Error bars indicate standard error of the mean of independent colonies (n=10). Statistical significance was determined by unpaired two-sided t-test. (E-F) Representative phenotypes of the reprogrammed iPSC from the donor somatic cells from Figure S6A and S6B from mouse and human donors. A-SC-AG4 somatic cells with the reduced ROS by the treatment of the glutathione reduced ethyl ester in FIGS. 7B and 7D was studied. (G) Immunoblot of pATM showing that A-iPSC with glutathione treatment recover the DNA damage response in the biologically independent clones after phleomycin treatment. A-iPSC were generated with the treatment of 3 mM glutathione reduced ethyl ester prior to and during the early stage of reprogramming (from one day before reprogramming virus infection to 10 days post reprogramming virus infection). (H) Copy number profiling analysis of human A-iPSC with glutathione treatment from 10 clones (upper panel). Schematic diagrams represent 10 non-rearranged A-iPSC with glutathione treatment, compared with seven rearranged A-iPSC from 11 clones (lower panel) without glutathione treatment in FIG. 5E. A-hiPSC (n=11 (7/11), p=0.64) and A-hiPSC-glutathione (n=10 (0/10), p*<4E-5). The p values are the observed (p) and estimated likelihoods (p*) of detecting no rearrangements in the absence of lineage effects using a binomial distribution, respectively. (I) Q-PCR of ZSCAN10. Error bars indicate standard error of the mean of two technical replicates with independent clones in each sample group in FIG. 7G. Statistical significance was determined by two-sided t-test. (J) Q-PCR of GSS. Error bars indicate standard error of the mean of two technical replicates with independent clones in each sample group in FIG. 7G. Statistical significance was determined by two-sided t-test.

FIG. 8. Immunoblot of pATM showing recovery of the DNA damage response after phleomycin treatment in ten independent clones of A-iPSC with BSO (0.5 mM)-mediated inhibition of GSS.

FIGS. 9A-9C. FIG. 9A shows an HT12 Illumina Mircroarray gene expression analysis between Aged somatic cells (A-SC (AG4)) and Young somatic cells (Y-SC (MRCS)). FIG. 9B lists the differentially expressed genes between Aged somatic cells (AG4) and Young somatic cells (MRCS). FIG. 9C is a pie chart dividing the differentially expressed genes between the Aged somatic cells and Young somatic cells based on molecular function. One gene, PRDX2, was found to be involved in redox regulation (GO:0016209).

FIG. 10. FIG. 10 is a table comparing the reprogramming efficiency for AG4 cells and AG4 cells treated with glutathione reduced ethyl ester according to the methods of the present technology.

FIG. 11. FIG. 11 demonstrates the variation of oxidative stress among the human population, and oxidative stress control of the G-quadruplex DNA structure. Basal oxidative stress levels in somatic cells (fibroblasts from different human tissue donors; 80-100 years of age) were measured by MitoSOX RED staining.

FIG. 12A-12B. FIG. 12 shows a metabolic profiling analysis of the top 50 differential metabolites. Eleven dermal fibroblasts were randomly selected from aged human donors with high, intermediate, and low oxidative stress. All samples were analyzed in a mass-spectrometry based analysis using both, positive (FIG. 12A) and negative (FIG. 12B) heated electrospray ionization.

FIG. 13A-13B. FIG. 13 shows the variation of oxidative stress among the human population, and oxidative stress control of the G-quadruplex DNA structure. FIG. 13A demonstrates higher G4 IHC staining of fibroblasts with higher ROS and lower G4 IHC staining of fibroblasts with lower ROS. FIG. 13B demonstrates the quantification of G4 IHC staining.

FIG. 14A-14B. FIG. 14 shows the variation of oxidative stress among the human population, and oxidative stress control of the G-quadruplex DNA structure. FIG. 14A demonstrates lower G4 IHC staining of fibroblasts after treatment with glutathione to reduce ROS. Statistical significance was determined by two-sided t-test. FIG. 14B demonstrates higher G4 IHC staining of fibroblasts after treatment with BSO to increase ROS. Statistical significance was determined by two-sided t-test.

FIG. 15A-15B. FIG. 15 shows G4 profiling signatures on enhancer regions. Comparative G4-antibody based ChIP-seq was performed with somatic cells with high and low oxidative stress. Elevation of oxidative stress is associated with a significant reduction of G4 markers on enhancer regions. FIG. 15A shows samples with high oxidative stress and reduction of oxidative stress by GSH [3 mM, 4 hours] treated somatic cells in high oxidative stress. FIG. 15B shows samples with low oxidative stress and elevation of oxidative stress by BSO [0.5 mM, 4 hours] treated somatic cells in low oxidative stress.

FIG. 16A-16B. FIG. 16 shows an IHC-based 8-oxo-guanine (oxoG) quantification using a specific antibody. FIG. 16A shows higher oxoG IHC staining of A-SC with higher ROS vs. lower ROS. FIG. 16B shows the quantification of oxoG IHC staining by ImageJ software analysis in 3 independent clones with multiple replicate samples. Statistical significance was determined by two-sided t-test

FIG. 17A-17B. FIG. 17A is a table summarizing the positions of G4 structures in pluripotency genes. Figure discloses SEQ ID NOs: 19-26, in order of appearance. FIG. 17B shows quantitative PCR for pluripotency genes OCT4, KLF4, ZSCAN10, LIN28A, SOX2, CMYC, NANOG, and for the gene LIN28B in human ES after 72 hours of the G4 structure stabilizer PYRIDOSTATIN with various concentrations.

FIG. 18. FIG. 18 is a table summarizing the 27 potential genes upstream of ROS that were not influenced by GEE/BSO treatment.

FIG. 19A-19B. FIG. 19A demonstrates MitoSOX Red staining of control fibroblasts with high ROS and fibroblasts with high ROS treated with shCHI3. FIG. 19B demonstrates the quantification of the MitoSOX Red staining.

FIG. 20A-20B. FIG. 20 shows a proposed model on the development of radiation/chemotherapy resistance. FIG. 20A shows a mechanism by which elevated glutathione overcomes the inhibitory function of oxidative stress-associated somatic cell epigenetic markers, leading to the development of iPSC or Tumour initiating cells with radiation/chemotherapy resistance and higher tumorigenicity. FIG. 20B shows high and low ROS fibroblasts will be mixed with melanoma organoids, followed by monitoring of melanoma cancer progression, elevation of glutathione, and radiation/chemotherapy agent resistance in vitro and in vivo.

### DETAILED DESCRIPTION

It is to be appreciated that certain aspects, modes, embodiments, variations, and features of the present technology are described below in various levels of detail in order to provide a substantial understanding of the present technology.

### I. Definitions

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a,” “an,” and “the” include plural referents unless the content clearly dictates otherwise. For example, reference to “a cell” includes a combination of two or more cells, and the like.

As used herein, the term “about” encompasses the range of experimental error that may occur in a measurement and will be clear to the skilled artisan.

As used herein, the term “aged somatic cell” (abbreviated as A-SC) refers to a somatic cell isolated from an aged donor (e.g., a mouse aged ≥1.4 years, or a human aged ≥50 years) or exhibiting a profile (e.g., basal ROS level, DNA damage response, genomic stability, ZSCAN10 expression level, GSS expression level) that is comparable to a somatic cell isolated from an aged donor. Aged somatic cells include somatic cells, either isolated from an aged donor or exhibiting a profile comparable to a somatic cell isolated from an aged donor, which cannot generate iPSCs due to the inhibitory effects of high cellular ROS levels on pluripotent stem cell reprogramming. The term “aged-induced pluripotent stem cell” (abbreviated as A-iPSC) refers to an iPSC derived from a non-pluripotent cell, such as a somatic cell, isolated from an aged donor or exhibiting a profile (e.g., basal ROS level, DNA damage response, genomic stability, ZSCAN10 expression level, GSS expression level) that is comparable to an iPSC derived from a non-pluripotent cell, such as a somatic cell, isolated from an aged donor.

As used herein, the term “chromosomal structural abnormalities” refers to any change in the normal structure of a chromosome. Chromosomal structural abnormalities include, but are not limited to duplications, deletions, translocations, inversions, and insertions.

As used herein, the term “DNA damage response” refers to any process that results in a change in state or activity of a cell (in terms of movement, secretion, enzyme production, gene expression, etc.) as a result of a stimulus, indicating damage to its DNA from environmental insults or errors during metabolism.

As used herein, the term “differentiates” or “differentiated” refers to a cell that takes on a more committed (“differentiated”) position within a given cell lineage.

As used herein, an “effective amount” or a “therapeutically effective amount” of a compound refers to composition, compound, or agent levels in which the physiological effects of a disease or disorder are, at a minimum, ameliorated, or an amount that results in one or more desired outcomes in reprogrammed iPSCs including, but not limited to, increased reprogramming efficiency of non-pluripotent cells into iPSCs, improved genomic stability, improved DNA damage response, increased ZSCAN10 expression, and reduced glutathione synthetase (GSS) expression, relative to reprogrammed iPSCs that were not contacted with the composition, compound, or agent of the present technology. For example, a compound, such as glutathione or BSO, can be delivered to a cell or cell culture in an amount that results in one or more desired outcomes in reprogrammed iPSCs including, but not limited to, increased reprogramming efficiency of non-pluripotent cells into iPSCs, improved genomic stability, improved DNA damage response, increased ZSCAN10 expression, and reduced glutathione synthetase (GSS) expression, relative to reprogrammed iPSCs that were not contacted with the glutathione or BSO. A therapeutically effective amount can be given in one or more administrations (e.g., prior to initiation of reprogramming non-pluripotent cells to iPSCs, during reprogramming, after reprogramming, and/or throughout an iPSC culturing protocol as described herein). The amount of a compound which constitutes a therapeutically effective amount will vary depending on the compound, the disorder and its severity, and the general health, age, sex, body weight and tolerance to drugs of the subject to be treated, but can be determined routinely by one of ordinary skill in the art. For example, as described herein, an effective amount of an agent, such as glutathione reduced ethyl ester, can vary and be adjusted by the skilled artisan depending on a condition, such as the level of cellular reactive oxygen species (ROS), observed in a target population of non-pluripotent cells, such as somatic cells, obtained from a subject.

As used herein, “genomic instability” (also “genome instability” or “genetic instability”) refers to an increase in structural chromosomal alterations (e.g., deletions, amplifications, translocations), numerical chromosomal aneuploidy, or mutations on DNA sequence within the genome of a cellular lineage.

As used herein, “glutathione” encompasses glutathione derivatives and stabilized forms of glutathione, such as glutathione reduced ethyl ester (“GSH” or “GEE”).

As used herein, the term “induced pluripotent stem cells” (iPSCs) has a meaning well-known in the art and refers to cells having properties similar to those of embryonic stem cells (ESCs) and encompasses undifferentiated cells artificially derived by reprogramming differentiated, non-pluripotent cells, typically adult somatic cells.

As used herein, the term “oncogenic potential” means the likelihood that a cell after its transplantation into a host will generate malignant tumors in the host. The term is applied for example to induced pluripotent stem cells (iPSCs), and to their propensity to generate malignant tumors upon differentiation and transplantation into an animal or human. Phenotypic traits such as genomic instability and impaired DNA damage response indicate elevated oncogenic potential regardless of whether the iPSC has been derived from an aged donor.

As used herein, the term “pluripotent stem cell” (PSC) refers to a cell capable of continued self-renewal, and, under appropriate conditions, of producing progeny of several different cell types. PSCs are capable of producing progeny that are derivatives of each of the three germ layers: endoderm, mesoderm, and ectoderm, according to a standard art-accepted test, such as the ability to form a teratoma in a suitable host, or the ability to differentiate into cells stainable for markers representing tissue types of all three germ layers in culture. Included in the definition of PSCs are embryonic cells of various types, such as embryonic stem cells (ESCs), as well as induced pluripotent stem cells (iPSCs) that have been reprogrammed from non-pluripotent cells, such as adult somatic cells.

Those skilled in the art will appreciate that except where explicitly required otherwise, PSCs include primary tissue and established lines that bear phenotypic characteristics of PSCs, and derivatives of such lines that still have the capacity of producing progeny of each of the three germ layers. PSC cultures are described as “undifferentiated” or “substantially undifferentiated” when a substantial proportion of stem cells and their derivatives in the population display morphological characteristics of undifferentiated cells, clearly distinguishing them from differentiated cells of embryo or adult origin. Undifferentiated PSCs are easily recognized by those skilled in the art, and typically appear in the two dimensions of a microscopic view with high nuclear/cytoplasmic ratios and prominent nucleoli. It is understood that colonies of undifferentiated cells within the population will often be surrounded by neighboring cells that are differentiated.

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, the term “reprogramming” and grammatical equivalents refer to a process that alters or reverses the differentiation status of a somatic cell that is either partially or terminally differentiated. Reprogramming of a somatic cell may be a partial or complete reversion of the differentiation status of the somatic cell. In some embodiments, reprogramming is complete when a somatic cell is reprogrammed into an induced pluripotent stem cell. However, reprogramming may be partial, such as reversion into any less differentiated state. For example, reverting a terminally differentiated cell into a cell of a less differentiated state, such as a multipotent cell.

As used herein, “reprogramming efficiency” refers to the number of iPSC colonies generated per somatic or donor input cell. For example, reprogramming efficiency can be provided by the ratio between the number of donor cells receiving the full set of reprogramming factors and the number of reprogrammed colonies generated.

As used herein, the term “reprogramming factor” refers to a molecule, such as a transcription factor, which when contacted with a cell (e.g., expressed by a cell, transformed into a cell for expression, exogenously provided to a cell, etc.), can, either alone or in combination with other molecules, cause reprogramming (e.g., reprogram somatic cells to cells with a pluripotent state). By way of example, but not by way of limitation, reprogramming factors include, but are not limited to Oct3 protein, Oct4 protein, Myo-D-Oct4 (M₃O) protein, Sox1 protein, Sox2 protein, Sox3 protein, Sox15 protein, Klf1, protein, Klf2 protein, Klf3 protein, Klf4 protein, Klf5 protein, c-Myc protein, L-Myc protein, N-Myc protein, Nanog protein, Lin28A protein, Tert protein, Utf1 protein, Aicda protein, Glisl, Sa114, Esrrb, Tet1, Tet2, Zfp42, Prdm14, Nr5a2, Gata6, Sox7, Pax1, Gata4, Gata3, cEBPa, HNF4a, GMNN, SNAIL, Grb2, Trim71, and biologically active fragments, analogues, variants, and family members thereof. In some embodiments, the reprogramming factors comprise Oct4, Sox2, Klf4, and c-Myc (also known as the Yamanaka reprogramming factors). In some embodiments, Nanog and Lin28 replace Klf4 and c-Myc; esrrb replaces Klf4; SV40 LT (T) replaces Klf4, c-Myc, Lin28, and Nanog; BIX-01294 replaces Sox2 and Oct4; and VPA replaces Klf4 and c-Myc.

As used herein, the term “somatic cell” refers to any cell other than pluripotent stem cells or germ cells. In some embodiments, the cells may be any type of somatic cells, of any origin, including cells derived from humans or animals. By way of example, but not by way of limitation, somatic cells may include, but are not limited to fibroblast cells, epithelial cells, osteocytes, chondrocytes, neurons, muscle cells, hepatic cells, intestinal cells, spleen cells, and adult stem cells, including, but not limited to hematopoietic stem cells, vascular endothelial stem cells, cardiac stem cells, muscle-derived stem cells, mesenchymal stem cells, epidermal stem cells, adipose-derived stem cells, intestinal stem cells, neural stem cells, germ line stem cells, and hepatic stem cells.

As used herein, the terms “subject,” “individual,” or “patient” can be an individual organism, a vertebrate, a mammal, or a human.

“Treating,” “treat,” “treated,” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In the context of cells, cell cultures, tissues, and tissue cultures, the terms “treating,” “treat,” “treated,” or “treatment” as used herein covers contacting cells, cell cultures, tissues, or tissue cultures with an agent, such as glutathione reduced ethyl ester, and describes cells, cell cultures, tissues, and tissue cultures that have been contacted with the agent.

It is also to be appreciated that the various modes of treatment or prevention of medical diseases and conditions as described are intended to mean “substantial,” which includes total but also less than total treatment or prevention, and wherein some biologically or medically relevant result is achieved.

As used herein, the term “young somatic cell” (abbreviated as Y-SC) refers to a somatic cell isolated from a young donor (e.g., a mouse aged ≤5 days, or a human aged ≤16 years) or exhibiting a profile (e.g., basal ROS level, DNA damage response, genomic stability, ZSCAN10 expression level, GSS expression level) that is comparable to a somatic cell isolated from a young donor. The term “young-induced pluripotent stem cell” (abbreviated as Y-iPSC) refers to an iPSC derived from a non-pluripotent cell, such as a somatic cell, isolated from a young donor or exhibiting a profile (e.g., basal ROS level, DNA damage response, genomic stability, ZSCAN10 expression level, GSS expression level) that is comparable to an iPSC derived from a non-pluripotent cell, such as a somatic cell, isolated from a young donor.

### II. Methods for Generating iPSCs

### A. General

In one aspect, the present disclosure provides methods for producing induced pluripotent stem cells (iPSCs) from non-pluripotent cells. In some embodiments, the methods include culturing the non-pluripotent cells with an effective amount of glutathione prior to the initiation of reprogramming, during reprogramming, and/or following reprogramming to produce genome-stable iPSCs. In some embodiments, the methods of the present technology include producing iPSCs characterized by one or more of increased genomic stability, increased DNA damage response, increased ZSCAN10, reduced GSS expression, and/or increased reprogramming efficiency, as compared to that observed in iPSCs generated from control untreated non-pluripotent cells. In some embodiments, the methods of the present technology allow for reprogramming of non-pluripotent cells, such as aged somatic cells, which are otherwise resistant to reprogramming and/or generate iPSCs at low efficiency, if at all.

In another aspect, the present disclosure provides iPSCs and somatic cells differentiated from these iPSCs. For example, the iPSCs can be produced from somatic cells derived from a donor displaying an aged profile (A-iPSC), which may result from the aging process and/or lifestyle factors that contribute to an aged phenotype (e.g., smoking, excessive alcohol intake), and used to generate histocompatible transplantable tissue.

Despite advances in developing reprogramming methods and identifying additional reprogramming factors (e.g., ZSCAN10 as described below), the potential application of iPSC technology in clinical and research settings is hampered by the relatively low efficiency of iPSC generation, the requirement for the introduction of exogenous nucleic acids, and the genomic instability of A-iPSCs, which can contribute to an elevated oncogenic potential in the A-iPSCs.

One advantage of the present methods is that iPSCs can be generated from non-pluripotent cells (e.g., somatic cells) without the need for the addition of an exogenous nucleic acid or genetic modification beyond those introduced by the factors typically employed for iPSC reprogramming (e.g., Yamanaka factors Oct4, Sox2, Klf4, and c-Myc). The ability to reprogram cells, such as those derived from aged tissues, without the need for an additional nucleic acid transfection may be particularly advantageous in the clinical setting. In addition, the use of biomarkers that predict the genomic stability of derived iPSCs can assist the clinician in identifying non-pluripotent cells for treatment with glutathione according to the methods described herein. For example, increased cellular reactive oxygen species (ROS) levels observed in non-pluripotent cells, such as aged somatic cells, relative to those observed in control aged somatic cells, young somatic cells, or embryonic stem cells (ESCs) can be used to identify non-pluripotent cells for treatment with glutathione. In some embodiments, ZSCAN10 and/or GSS expression levels observed in non-pluripotent cells, such as aged somatic cells, relative to those observed in control aged somatic cells, young somatic cells, or ESCs can be used to identify non-pluripotent cells for treatment with glutathione. Accordingly, gene expression levels of GSS and/or ZSCAN10 and metabolite levels of ROS and/or glutathione can serve as biomarkers to predict the genomic stability in reprogrammed iPSCs. In addition, biomarkers, such as an increased Prdx2 expression in non-pluripotent cells that generate genome-unstable A-iPSCs relative to that observed in non-pluripotent cells that generate genome-stable A-iPSCs, can also be used to identify non-pluripotent cells for treatment with glutathione. Thus, another advantage of the methods of the present technology is that the use of biomarkers, such as cellular ROS levels, ZSCAN10/GSS expression levels, and Prdx2 expression levels, will reduce or eliminate additional testing to assess the clinical quality of A-iPSCs. For example, based on the characteristics of the non-pluripotent cells (e.g., aged somatic cells), the reprogramming protocol can be tailored (e.g., using the standard Yamanaka factors with or without glutathione treatment) to increase efficiency and produce genome-stable iPSCs.

### B. A-iPSC Show Impaired Genomic Integrity and are Defective in Apoptosis and DNA Damage Response Compared to Y-iPSC and ESC

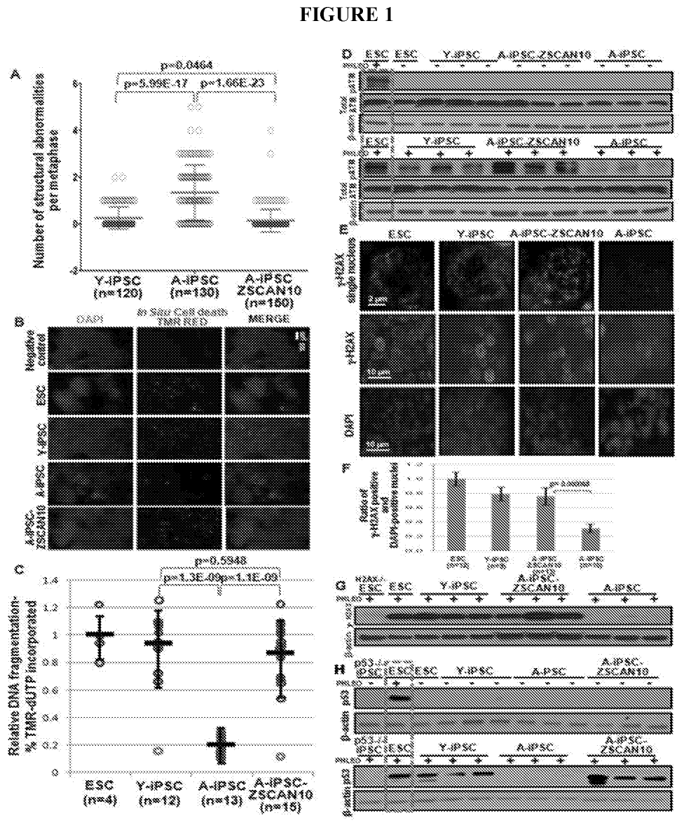

Y-iPSC (using mouse skin fibroblasts from E17.5 embryos to 5-day-old neonates) and A-iPSC (using mouse skin fibroblasts from 1.5-year-old adults) were generated as described previously (Takahashi & Yamanaka, Cell 126(4):663-676 (2006)). A minimum of 12 iPSC clones was randomly selected to undergo a series of common pluripotency tests previously used to characterize mouse and human iPSC including teratoma/chimera analysis and pluripotent gene expression analysis. Q-PCR analysis of these clones was performed to confirm silencing of the reprogramming factors. All clones passed the panel of pluripotency tests; however, as shown in FIG. 1A, cytogenetic analysis revealed a greater number of chromosomal structural abnormalities in A-iPSC (n=130) compared to Y-iPSC (n=120).

A-iPSC showed better survival following manipulative stress, such as passaging and thawing, compared to Y-iPSC or ESC. As demonstrated by FIGS. 1B and 1C, an in situ cell death assay revealed that Y-iPSC (n=12) and ESC controls (n=4) showed a significant level of apoptosis after treatment with phleomycin (a structural analogue of bleomycin with higher potency). In contrast, A-iPSC (n=13) showed a poorer apoptotic response to phleomycin compared to either Y-iPSC or ESC. This indicated that a defect in the apoptotic response to DNA damage in A-iPSC would result in a greater number of cells with genetic abnormalities, reflecting a defect in the elimination of damaged cells.

As shown in FIGS. 1D-1G, compared to Y-iPSCs or ESCs, A-iPSCs consistently exhibit poor activation of the ATM-H2AX-p53 pathway, indicating that the normal cellular mechanisms involved in the DNA damage response are attenuated in A-iPSCs, leading to a failure to eliminate cells with aberrant genomic content. The poor DNA damage response in A-iPSCs has been shown to persist during extended tissue culture (up to passage 19). A-iPSCs generated from two additional tissue types (lung and bone marrow) have also been shown to exhibit similar defects in the DNA damage response.

### C. ZSCAN10 Recovers the DNA Damage Response and Genomic Stability of Mouse A-iPSC

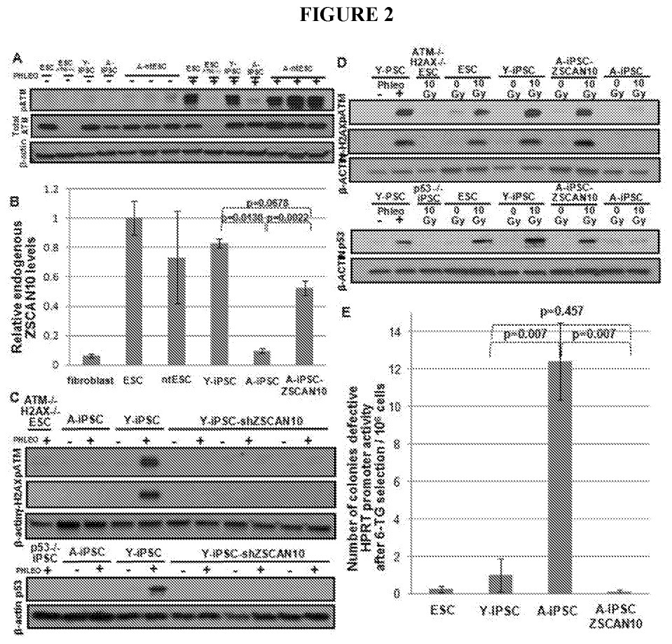

Nuclear transfer is an alternative reprogramming method to create patient-specific pluripotent stem cells (ntESC). Mouse ntESCs were generated by inserting nuclei from aged tissue donors into enucleated oocytes to produce A-ntESCs. As shown in FIG. 2A, unlike A-iPSCs, the A-ntESCs showed a normal DNA damage response with a normal cytogenetic signature. Because oocytes likely contain other reprogramming factors in addition to the four Yamanaka factors employed to generate iPSCs, additional pluripotency factors—present in the enucleated oocyte but absent from aged somatic cells—may be required for a normal DNA damage response. Such factors may also be present in Y-iPSC and ESC because they have a normal DNA damage response.

ZSCAN10, a known zinc finger transcription factor specifically expressed in ESC has been identified. ZSCAN10 is an integrated part of the transcriptional regulatory network with SOX2, OCT4, and NANOG. Time-lapse imaging experiments in fibroblasts have shown that ZSCAN10 expression was detectable starting on day 6 of reprogramming and was strongly expressed at the time iPSC colonies were formed.

In addition, as shown in FIG. 2B, endogenous ZSCAN10 expression is high in Y-iPSC and ESC, but low in A-iPSC. Expression of ZSCAN10 with a doxycycline-inducible promoter during reprogramming days 5 through 14 in A-iPSC (A-iPSC-ZSCAN10) persistently increased endogenous ZSCAN10 expression to levels similar to those in Y-iPSC and ESC (FIG. 2B), and expression has been shown to be stable up to passage 15. As shown in FIG. 1A, A-iPSC-ZSCAN10 (n=150) have a reduced number of chromosomal structure abnormalities, comparable to the frequency seen in Y-iPSC and ESC. A-iPSC-ZSCAN10 clones also showed recovery of apoptosis (FIG. 1B, 1C) and the DNA damage response (FIG. 1D-1G). This recovery was not due to a slower DNA damage response, slower growth rate, or differential telomere length in A-iPSC-ZSCAN10 compared to A-iPSC. Conversely, as shown in FIG. 2C, downregulation of ZSCAN10 via shRNA during reprogramming in Y-iPSC has been shown to impair the DNA damage response.

Although the majority of Y-iPSC and A-iPSC-ZSCAN10 clones show a higher apoptotic response compared to A-iPSC, two outlier clones did not show a restoration of the apoptotic response (FIG. 1C) and were found to have chromosome abnormalities (data not shown). These outlier clones had low ZSCAN10 expression and a defective DNA damage response (data not shown), providing further support that ZSCAN10 is a positive regulator of genomic stability through the induction of apoptosis in response to DNA damage. The defective DNA damage response of A-iPSC and its restoration by ZSCAN10 were also confirmed in iPSC exposed to other DNA damaging agents such as radiation (FIG. 2D).

The failure to eliminate A-iPSC with DNA damage via apoptosis leads to the accumulation of genomic mutations in A-iPSC compared to either Y-iPSC or ESC. As shown in FIG. 2E, as assessed by the HPRT mutation assay (which measures the mutagenic destruction of HPRT promoter activity), relative to ESC and Y-iPSC, A-iPSC had the highest mutagenicity, which was recovered by ZSCAN10 expression.

### D. ZSCAN10 Restores ROS-Glutathione Homeostasis in Mouse A-iPSC Via Reduction of Excessively Activated GSS

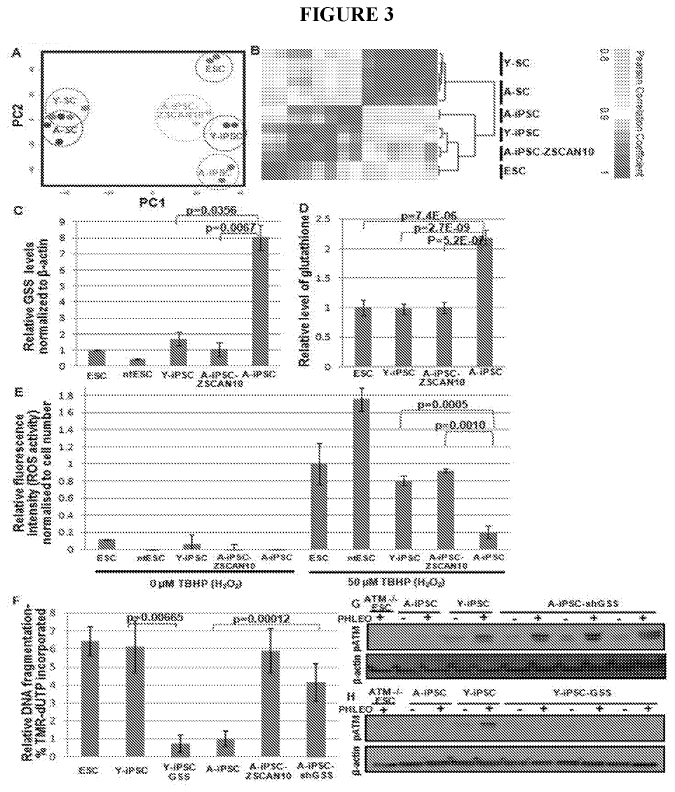

Transient expression of ZSCAN10 as a pluripotent transcription factor during reprogramming in A-iPSC has been shown to the overall pluripotent transcriptional regulatory network to resemble to that of Y-iPSC (FIGS. 3A, 3B). To identify ZSCAN10 targets involved in the DNA damage response defect in A-iPSC, ZSCAN10 targeted promoter binding regions from those previously reported in ChIP-on-Chip analysis in ESC were cross-referenced with lists of (1) differentially expressed genes in Y-iPSC/ESC and A-iPSC, (2) genes with altered expression in A-iPSC compared to A-iPSC-ZSCAN10, and (3) genes with known functions in the DNA damage response and genomic stability. The resulting genes were further narrowed down by confirming their expression patterns in human A-iPSC, ESC, Y-iPSC, and A-iPSC-ZSCAN10 by Q-PCR. This stringent, multi-step analysis identified glutathione synthetase (GSS), which was expressed at excessively high levels in A-iPSC but was downregulated upon ZSCAN10 expression in A-iPSC, to the levels seen in Y-iPSC or ESC (FIG. 3C). Conversely, downregulation of ZSCAN10 by shRNA in Y-iPSC led to elevated GSS expression, supporting a role of ZSCAN10 as a suppressor of GSS expression. ChIP-Q-PCR confirmed ZSCAN10 binding activity to the GSS promoter to suppress GSS expression (FIG. 3C).

A-iPSC have excessive levels of glutathione (FIG. 3D) and elevated ROS scavenging activity (FIG. 3E) relative to Y-iPSC or ESC. While ROS levels in A-iPSC were increased by treatment with DNA damaging agents (FIG. 3E) and this might be sufficient to cause direct DNA damage and genomic instability, improper scavenging of ROS by excess glutathione would limit the ROS cellular stress signal needed to induce the DNA damage response, which would in turn reduce apoptosis and increase A-iPSC exposure to additional genotoxic stress, allowing accumulation of mutations and other genomic alterations. Upon ZSCAN10 expression, glutathione and ROS scavenging activity were normalized to levels equivalent to those seen in Y-iPSC and ESC (FIG. 3D, 3E). In addition, shRNA knockdown of GSS in reprogrammed A-iPSC (data not shown) decreased glutathione levels and ROS scavenging activity (data not shown), increased apoptosis (FIG. 3F), and recovered the DNA damage response (FIG. 3G). The DNA damage response was also recovered by treatment of A-iPSC with the GSS pharmacological inhibitor, L-Buthionine-sulfoximine (BSO) (data not shown). Conversely, overexpression of GSS in Y-iPSC (data not shown) increased glutathione and ROS scavenging activity (data not shown), decreased apoptosis (FIG. 3F), and blunted the DNA damage response (FIG. 311).

### E. ZSCAN10 Recovers the DNA Damage Response in Human A-hiPSC Caused by Excessive GSS

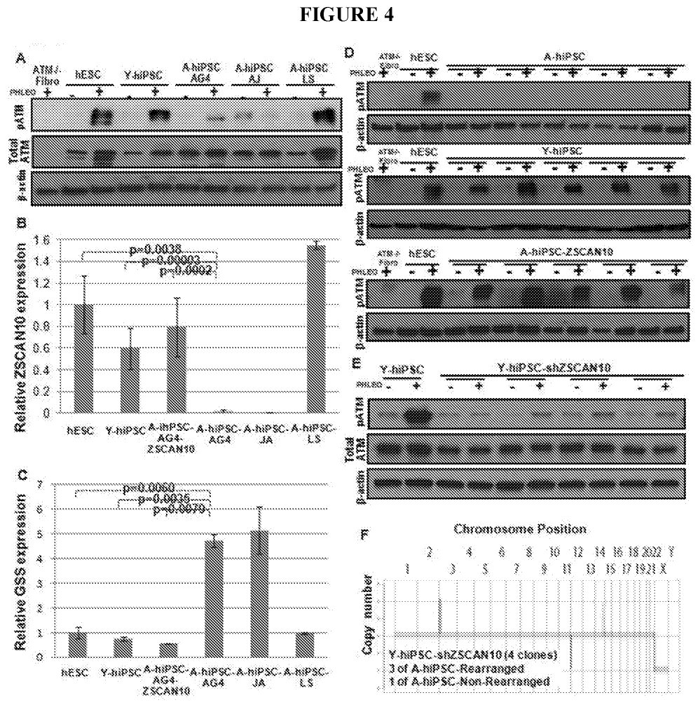

Consistent with the phenotypes observed in mouse A-iPSC, A-hiPSC-JA and A-hiPSC-AG4 showed a poor DNA damage response (FIG. 4A), low levels of ZSCAN10 (FIG. 4B), high levels of GSS (FIG. 4C), and genomic instability (see, e.g., Prigione, et al. PloS one 6(11):e27352 (2011)). However, A-hiPSC-LS did not exhibit these aging phenotypes and had a normal DNA damage response, normal ZSCAN10/GSS expression (FIGS. 4A-4C), and genomic stability (see, e.g., Miller, et al. Cell Stem Cell 13(6):691-705 (2013)). Similar clonal variation among human tissue donors was described in the recent A-hiPSC clinical trial (Garber, Nature Biotechnology 33(9):890-891 (2015); Coughlan, New Sci. 227(3033):9 (2015)). In that trial, treatment proceeded successfully with A-hiPSC generated from the first patient without significant genomic instability, but the trial was halted upon discovery of genomic instability in A-hiPSC generated from the second patient. A similar variability in A-iPSC derived from mice of different genetic backgrounds was observed: more A-iPSC clones from B6129 mice showed genomic stability with a normal DNA damage response, higher ZSCAN10 expression, and lower GSS expression (data not shown), compared to A-iPSC from B6CBA mice (FIGS. 1, 2B, 3C). Together, these observations underscore the idea that, even as mechanisms that contribute to the aging phenotype in A-iPSC are uncovered, differences in genetic polymorphisms and lifestyle play critical roles in aging and its biological effects on iPSC reprogramming in both mouse and human models.

The cross-species conservation of the mechanism that maintains ROS and glutathione homeostasis was analyzed using AG4 fibroblasts with a confirmed poor DNA damage response. A-hiPSC were generated in the presence and absence of human ZSCAN10 expression using a doxycycline system. Each A-hiPSC clone was put through a series of pluripotency tests and compared to hESC and Y-hiPSC derived from fibroblasts. As we observed in mouse A-iPSC, endogenous ZSCAN10 expression was significantly lower in A-hiPSC than Y-hiPSC or hESC (FIG. 4B). A-hiPSC also showed a blunted DNA damage response (pATM; FIG. 4D) and a poorer apoptotic response to phleomycin (data not shown) compared to Y-hiPSC or hESC. Poor DNA damage response in A-hiPSC was confirmed with various reprogramming vectors such as lentivirus reprogramming without MYC and an integration-free episomal vector system (data not shown), indicating that the observed phenotype of A-hiPSC is not caused by reprogramming vector systems or viral vector integration. As with the reprogramming of aged mouse donor cells, transient expression of ZSCAN10 during reprogramming days 5 through 15 in A-hiPSC (A-hiPSC-ZSCAN10) persistently increased endogenous Z SCAN10 expression to levels similar to those in Y-hiPSC and hESC (FIG. 4B). Increased ZSCAN10 expression recovered the DNA damage response (FIG. 4D) and the apoptosis defect (data not shown) in A-hiPSC. Also consistent with the mouse data, A-hiPSC express higher levels of GSS (FIG. 4C), which were normalized by increased expression of ZSCAN10 (FIG. 4C). Conversely, shRNA knockdown of ZSCAN10 in Y-hiPSC impaired the DNA damage response (FIG. 4E) and genomic stability (FIG. 4F). In addition, shRNA knockdown of ZSCAN10 in hiPSC generated from a previously reported secondary reprogramming system, in which H1 hESC-derived fibroblasts were reprogrammed into hiPSC (equivalent to Y-hiPSC) by pre-integrated doxycycline-inducible reprogramming lentivirus, impaired the DNA damage response (data not shown). ChIP-Q-PCR confirmed that ZSCAN10 directly binds to the ZSCAN10 DNA binding motif on the human GSS promoter (data not shown) to suppress GSS expression (FIG. 4C).

A-hiPSC had excessive levels of glutathione (FIG. 5A) and elevated ROS scavenging activity (FIG. 5B) relative to Y-hiPSC or hESC, as we observed in the mouse. Upon ZSCAN10 expression, glutathione and ROS scavenging activity were normalized to levels equivalent to those seen in Y-hiPSC and hESC (FIG. 5A, 5B). shRNA knockdown of GSS in A-hiPSC recovered the DNA damage response (FIG. 5C), while overexpression of GSS in Y-hiPSC blunted the DNA damage response (FIG. 5D). Together, these data confirmed the evolutionary conservation of a regulatory mechanism by which ZSCAN10 normalizes GSS levels and ROS/glutathione homeostasis, and recovers the DNA damage response in both two mouse and five human cell lines.

### F. ZSCAN10 Maintains Genomic Integrity in Human A-hiPSC

Chromosomal structural abnormalities (e.g., translocation, duplication, and deletion) in A-hiPSC clones were examined by a combination of DNA sequencing-based copy number variation analysis and karyotyping analysis to confirm the effect of ZSCAN10 on genomic instability. Seven A-hiPSC clones from eleven independent A-hiPSC clones showed a cytogenetic abnormality in eight regions (FIG. 5E), while five A-hiPSC-ZSCAN10 and ten Y-iPSC did not (FIG. 5F, 5G). We also observed sex chromosome aneuploidy in one A-hiPSC clone and trisomy 12 in one A-iPSC-ZSCAN10 clone, which are common chromosomal alterations in pluripotent stem cell culture and more likely to have been introduced by in vitro expansion and not by A-iPSC specific reprogramming.

Whole-exome sequencing analysis was performed in a randomly selected subset of the A-hiPSC (three clones with a normal cytogenetic signature and five clones with cytogenetic alterations), and A-hiPSC with ZSCAN10 expression (four clones) using the somatic cells as a reference genomic sequence to explore whether mutation rates are altered in A-hiPSC. Two dominant nonsynonymous point mutations and two synonymous mutations were uncovered (data not shown). Cytogenetic and point mutation analyses revealed that all A-hiPSC clones contain cytogenetic abnormalities or nonsynonymous point mutations, which were not observed in A-hiPSC-ZSCAN10 clones. Absence of common cytogenetic abnormalities or point mutations in fibroblasts used to generate A-iPSC was confirmed by karyotyping (screening 20 clones), chromosome painting (screening 100 clones), and whole-exome sequencing (80X coverage). Recurrent cytogenetic abnormalities or point mutations in independent clones of A-hiPSC may be induced either during iPSC reprogramming or exist in low frequency prior to reprogramming, which would give a selective reprogramming or growth advantage to aged cells. However, ZSCAN10 expression reduced the selective advantage of genomic alterations. In both mouse and human models (FIG. 1A, 2E, 5E, 5F), ZSCAN10 expression in A-hiPSC during reprogramming increased the likelihood of obtaining A-hiPSC with genomic stability. This human data confirm that the effect of ZSCAN10 on genomic instability is evolutionarily conserved, with ZSCAN10 recovering genomic stability in A-hiPSC and recapitulating what was seen in the mouse model.

Another member of the ZSCAN family, ZSCAN4, may help maintain genomic integrity of Y-iPSC and may function synergistically with ZSCAN10 in protecting the genome.

### G. Somatic Cell ROS as a Causative Origin of the Genomic Instability in A-iPSC and Recovery by Glutathione Treatment

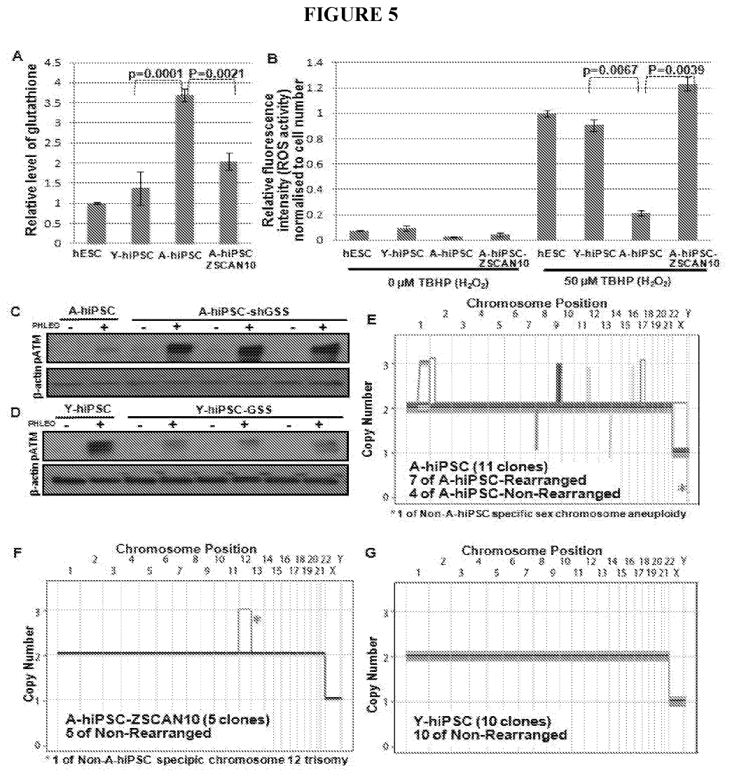

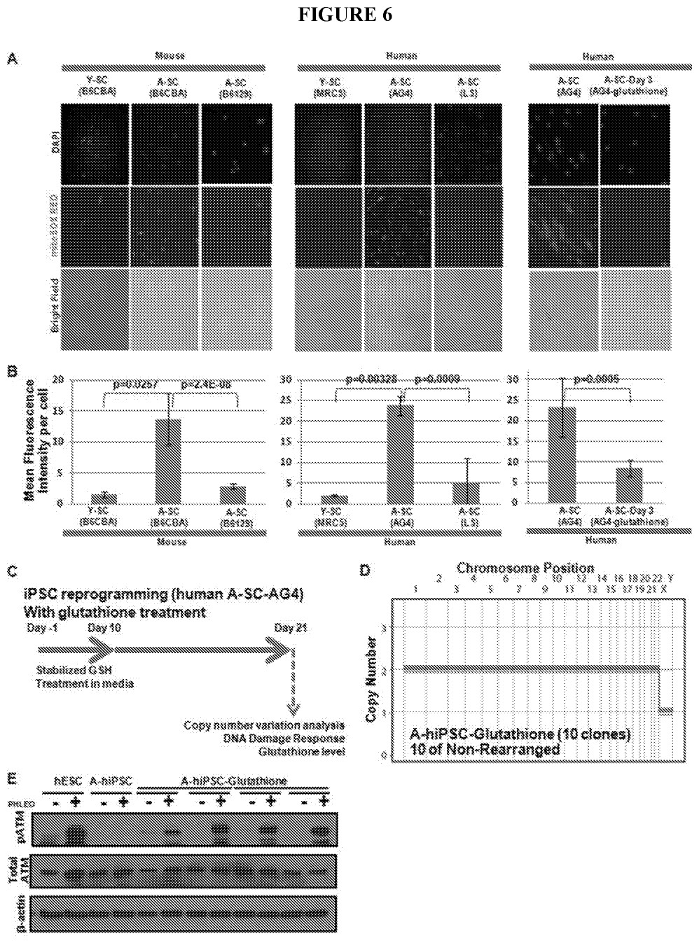

The main driver for the poor DNA damage response and genomic instability in A-iPSC and why some A-iPSC show a more pronounced aging phenotype are still unclear. Cellular ROS levels (detected by MitoSOX staining) correlate with a poor DNA damage response/genomic instability in human AG4 vs. LS somatic cells and B6CBA vs. B6129 mice (FIGS. 6A-6B). As described below, the effect of glutathione treatment (stabilized form of glutathione, 3 mM of glutathione reduced ethyl ester, CAT #G-275-500, GoldBio) of 10 human AG4 fibroblast clones, which show a higher level of MitoSOX staining, prior to and during the first 10 days of A-hiPSC reprogramming (FIG. 6C) was examined. As shown by FIG. 6, glutathione treatment according to the methods disclosed herein reduces the MitoSOX level, protects the DNA damage response (FIG. 6E), and maintains genomic stability (FIG. 6D) compared to untreated A-hiPSC (FIG. 5E).

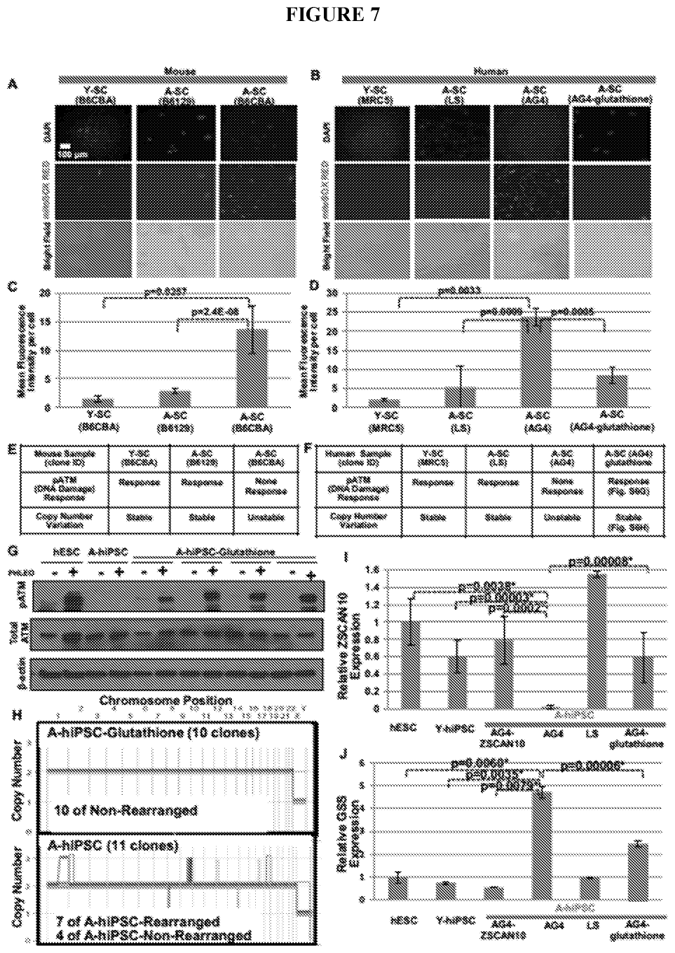

In addition, the methods disclosed herein can also generate A-iPSCs with increased genomic stability (FIG. 7H), increased DNA damage response (FIG. 7G), increased ZSCAN10 expression levels (FIG. 7I), and reduced GSS expression levels (FIG. 7J) as compared to that observed in A-iPSCs produced from untreated control aged somatic cells grown under similar conditions and/or comparable to that observed in Y-iPSCs or ESCs.

H. Sources of Cells for Reprogramming into iPSCs

The type and age of non-pluripotent cells (e.g., somatic cells) that may be reprogrammed into iPSCs by the methods disclosed herein are not limiting, and any kind of somatic cells may be used. In some embodiments, mature somatic cells may be used. In some embodiments, somatic cells are from an embryonic stage. In some embodiments, somatic cells are aged somatic cells. In some embodiments, the somatic cells are incapable of generating iPSCs. By way of example, but not by way of limitation, somatic cells may be primary cells (non-immortalized cells), such as those freshly isolated from an animal, or may be derived from a cell line (immortalized cells). In some embodiments, the somatic cells are mammalian cells, such as, for example, human cells or mouse cells. By way of example, but not by way of limitation, somatic cells may be obtained by well-known methods, from different organs, such as, but not limited to, skin, eye, lung, pancreas, liver, stomach, intestine, heart, reproductive organs, bladder, kidney, urethra and other urinary organs, or generally from any organ or tissue containing living somatic cells, or from blood cells. In some embodiments of the methods disclosed herein, fibroblasts are used. In some embodiments of the methods disclosed herein, cells isolated from the blood and/or bone marrow (which include, but are not limited to, endothelial cells, lymphocytes, myeloid cells, leukocytes, mesenchymal stem cells, and hematopoietic stem cells) are used. In some embodiments of the methods disclosed herein, mesenchymal stem cells are used. The term somatic cell, as used herein, is also intended to include adult stem cells.

i. Biomarkers Defining Elevated Cellular ROS Levels

In some embodiments, the non-pluripotent cells (e.g. somatic cells) are selected for treatment with glutathione or derivatives thereof based on the detection of oxidative stress-associated (i.e., elevated cellular ROS level) biomarkers in donor non-pluripotent stem cells (e.g., somatic cells). In some embodiments, the non-pluripotent cells (e.g. somatic cells) are selected for treatment with glutathione or derivatives thereof based on the detection of additional oxidative stress-associated biomarkers in the non-pluripotent cells that lead to aging phenotypes in A-iPSC and in tumor-initiating cells (TIC). For example, these markers may predict decreased reprogramming efficiency, elevated tumorigenicity, and/or the development of radiation/chemotherapy resistance in iPSCs generated from somatic cell donors or TIC in aged individuals. In some embodiments, the present technology relates to methods for characterizing the genomic stability of A-iPSC based on biomarkers of somatic cells from aged donors to tailor the reprogramming protocol (e.g., reprogramming somatic cells with the standard Yamanaka factors with or without glutathione treatment) to produce iPSCs characterized by one or more of increased genomic stability, increased DNA damage response, increased ZSCAN10 expression, and reduced glutathione synthetase (GSS) expression.

ROS

In some embodiments, the non-pluripotent cells (e.g., somatic cells) are selected for treatment with glutathione based on the expression level of cellular ROS in a sample of the non-pluripotent cells. For example, in some embodiments, aged somatic cells are selected for treatment with glutathione or derivatives thereof according to the methods of the present technology based on an elevated cellular ROS level prior to treatment relative to one or more of untreated control aged somatic cells, young somatic cells, and ESCs, wherein an elevated cellular ROS level identifies the aged somatic cells for treatment with glutathione or derivatives thereof and the lack of elevated cellular ROS level does not identify the aged somatic cells for treatment with ROS or derivatives thereof.

Prdx2

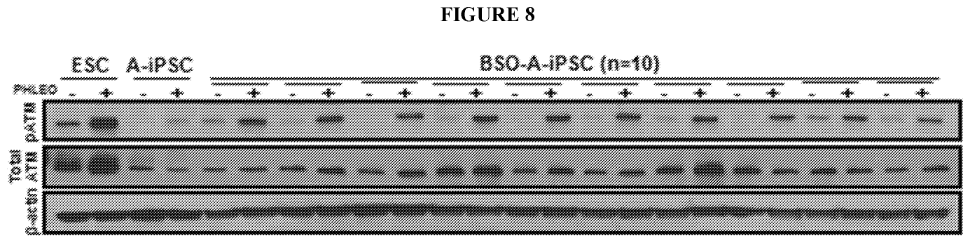

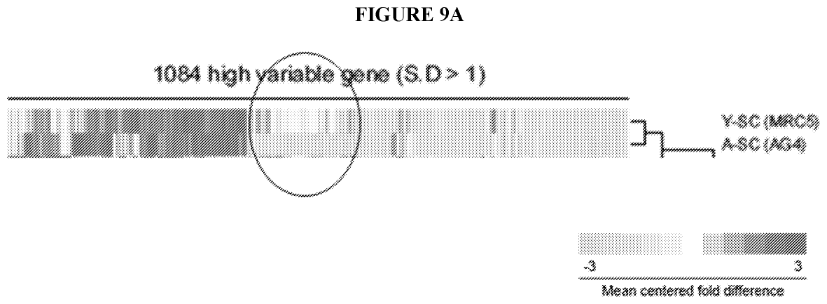

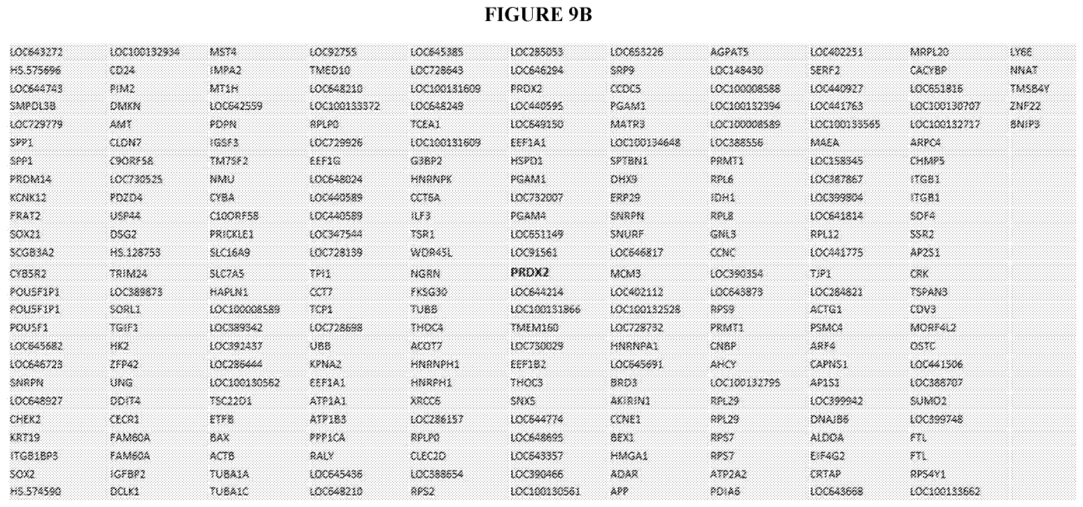

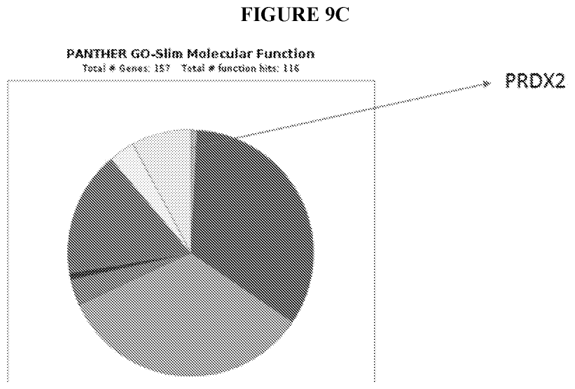

In some embodiments, the non-pluripotent cells (e.g. somatic cells) are selected for treatment with glutathione or derivatives thereof based on the expression level of a biomarker, such as Prdx2. For example, in some embodiments, aged somatic cells are selected for treatment with glutathione or derivatives thereof according to the methods of the present technology based on Prdx2 expression levels. Prdx2 was identified as a biomarker based on the results of a microarray gene expression analysis between Aged somatic cells and Young somatic cells (FIG. 9A). The results of the microarray analysis shown in FIG. 9A revealed 255 differentially expressed genes between an Aged somatic cell line (AG4) and a Young somatic cell line (MRCS) (FIG. 9B). Among the 255 differentially expressed genes, one gene, Prdx2, was found to be involved in redox regulation (GO:0016209) in the cell (FIG. 9C). Prdx2 levels were found to be 10 times higher in MRCS fibroblasts than in AG4 fibroblasts.

In some embodiments, cells are reprogrammed for an intended therapeutic use, and are derived from the patient subject (i.e., autologous). Somatic cells can be derived from a healthy or diseased subject. Somatic cells can be derived from a young donor (Y-SC) (e.g., a mouse aged ≤5 days, or a human aged ≤16 years) or exhibiting a profile (e.g., basal ROS level, DNA damage response, genomic stability, ZSCAN10 expression level, GSS expression level) that is comparable to a somatic cell isolated from a young donor. The term “young-induced pluripotent stem cell” (abbreviated as Y-iPSC) refers to an iPSC derived from a non-pluripotent cell, such as a somatic cell, isolated from a young donor or exhibiting a profile (e.g., basal ROS level, DNA damage response, genomic stability, ZSCAN10 expression level, GSS expression level) that is comparable to an iPSC derived from a non-pluripotent cell, such as a somatic cell, isolated from a young donor. In some embodiments, the somatic cells are derived from an aged donor (A-SC) (e.g., a mouse aged ≥1.4 years, or a human aged ≥50 years) or exhibiting a profile (e.g., basal ROS level, DNA damage response, genomic stability, ZSCAN10 expression level, GSS expression level) that is comparable to a somatic cell isolated from an aged donor. The term “aged-induced pluripotent stem cell” (abbreviated as A-iPSC) refers to an iPSC derived from a non-pluripotent cell, such as a somatic cell, isolated from an aged donor or exhibiting a profile (e.g., basal ROS level, DNA damage response, genomic stability, ZSCAN10 expression level, GSS expression level) that is comparable to an iPSC derived from a non-pluripotent cell, such as a somatic cell, isolated from an aged donor.

Metabolic Profiling (Metabolomics)

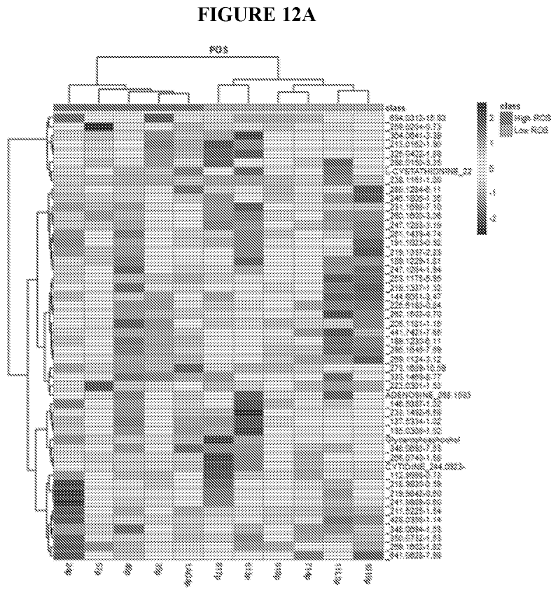

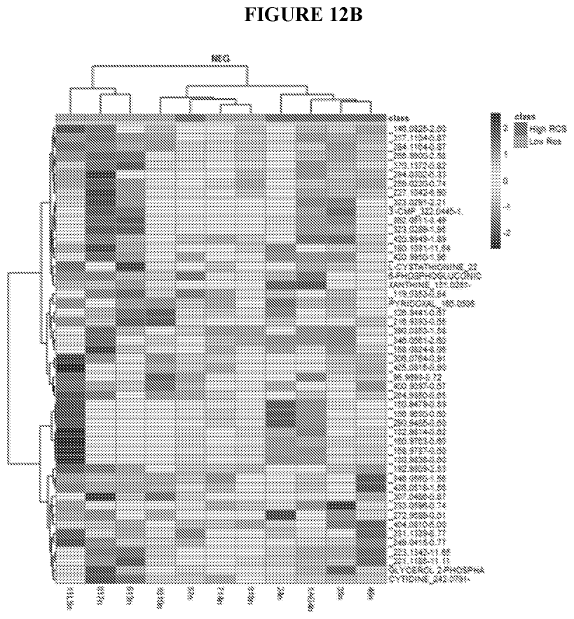

In some embodiments, the non-pluripotent cells (e.g., somatic cells) are selected for treatment with glutathione or derivatives thereof based on a metabolic profile. The results of the metabolic profiling analyses described herein demonstrate that cells (e.g., somatic fibroblasts) derived from aged donors have distinct profiles based on cellular ROS levels (FIGS. 12 A and 12B). The results of the metabolic profiling analysis comparing the metabolome from high ROS donor cells to low ROS donor cells revealed 41 significantly altered metabolites, four of which have been characterized. Donor somatic cells exhibiting a metabolic profile similar to that of high ROS control somatic cells may be selected for glutathione treatment. In some instances the measurement of a metabolic profile in donor somatic cells provides a method for detecting elevated ROS levels that is more stable than directly measuring cellular ROS levels.

G4 DNA Structure Formation and 8-Oxo-Guanine (oxoG) Formation

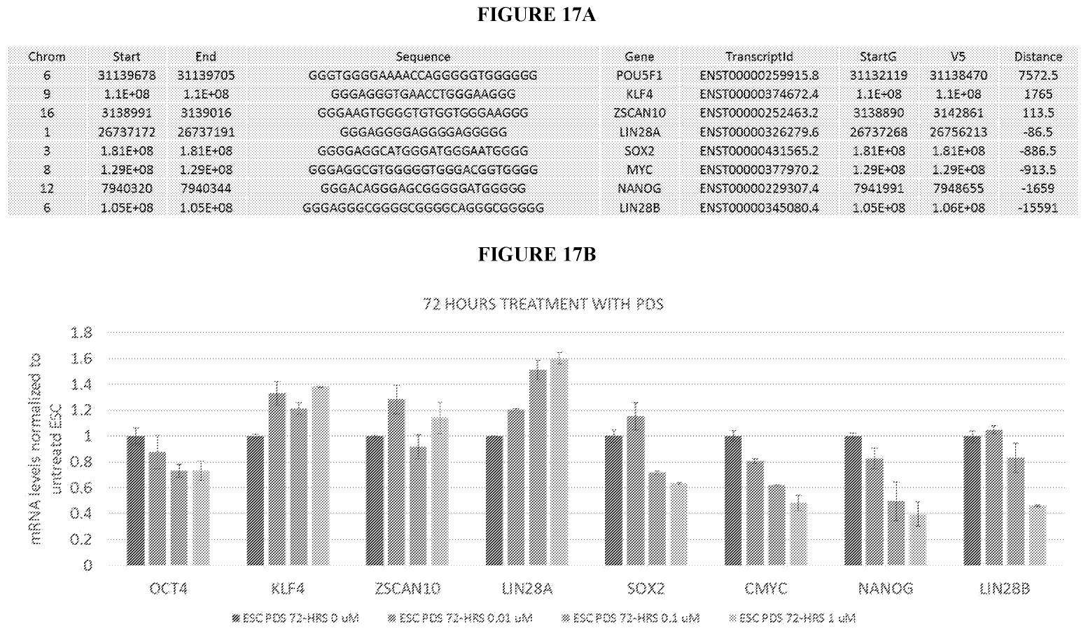

In some embodiments, the non-pluripotent cells (e.g., somatic cells) are selected for treatment with glutathione or derivatives thereof based on the levels of 8-oxo-guanine (oxoG) and guanine-quadruplex (G4) structure formation levels relative to those found in high ROS control somatic cells. G4 and oxoG are two examples of ROS-induced direct chemical alterations. Guanine is the primary oxidation target of ROS, which generates oxoG. This stabilizes a three-dimensional G4 structure on promoter regions that inhibits gene expression. The G4 structure is located in the regulatory regions of several pluripotent genes including SOX2, CMYC, NANOG, and others (FIGS. 17A and 17B).

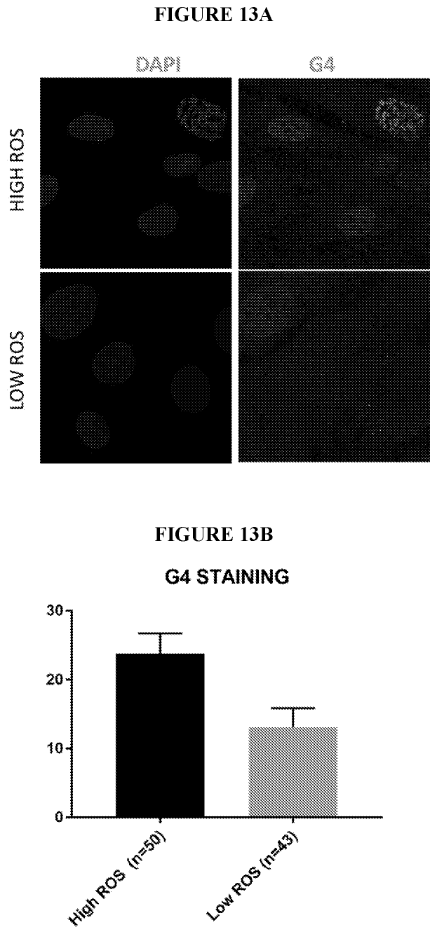

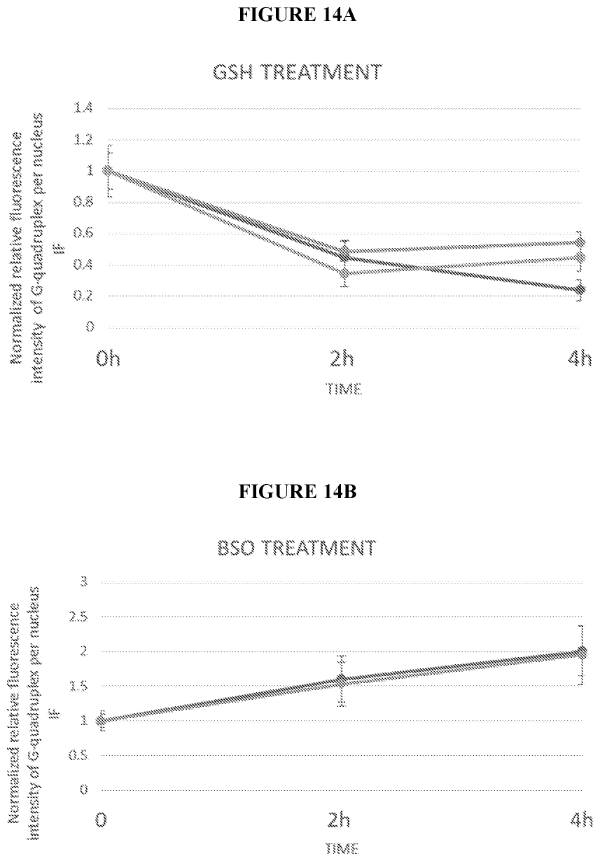

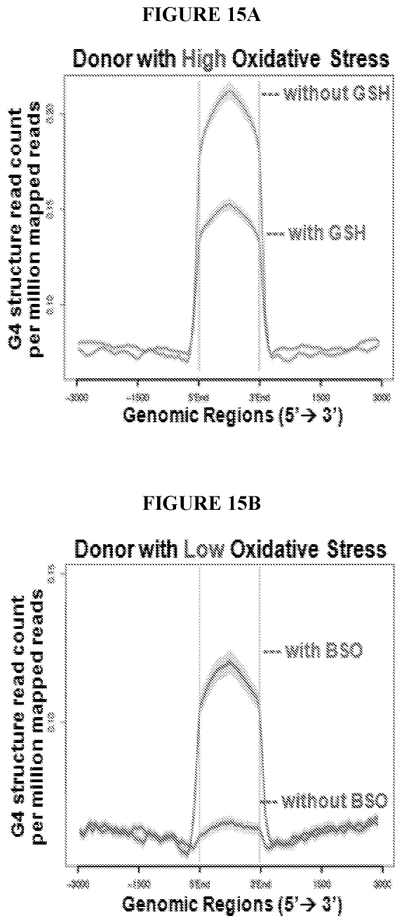

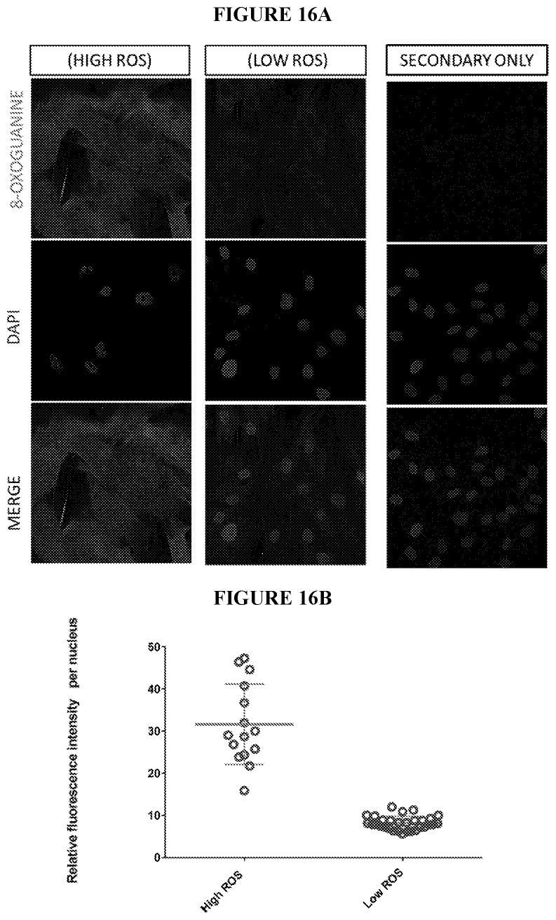

As described herein, G4 structure formation (FIGS. 13A, 13B, 15A, and 15B) and oxoG formation (FIGS. 16A and 16B) are elevated in high ROS cells relative to low ROS cells. Accordingly, G4 structure formation and/or oxoG formation can be used as an evaluation tool for detecting DNA damage resulting from ROS. In some instances the measurement of G4 structure formation and/or oxoG formation in somatic donor cells provides a method for detecting DNA damage resulting from ROS that is more stable than directly measuring cellular ROS levels.

Transcriptome Analysis

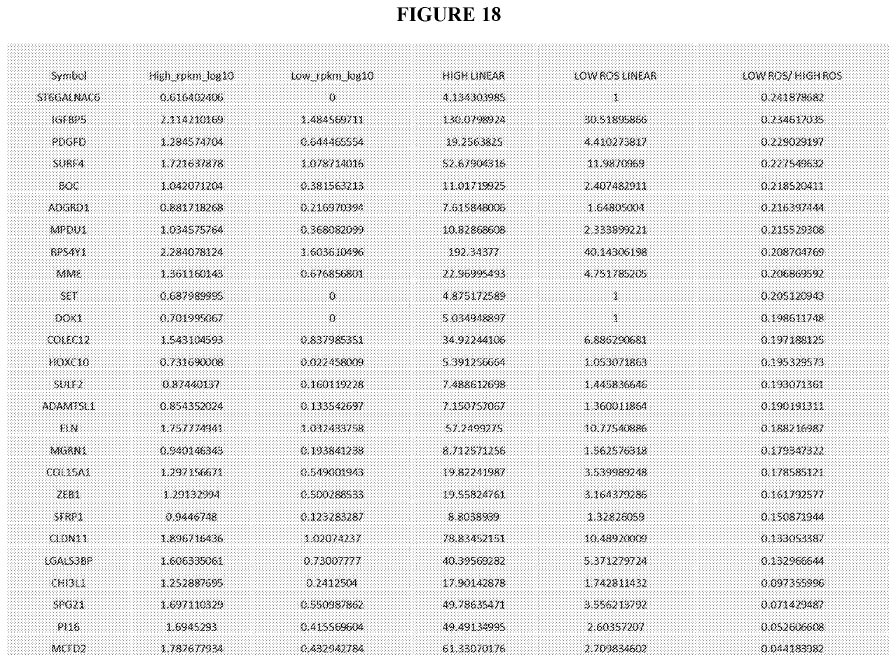

In some embodiments, the non-pluripotent cells (e.g., somatic cells) are selected for treatment based on a transcriptome profile. The results of the transcriptome analyses described herein demonstrate that a panel of genes functioning as upstream regulators of ROS formation exhibit altered gene expression in somatic cells derived from aged donors (FIG. 18).

In some embodiments, the present technology relates to the use of methods decreasing cellular ROS levels by suppressing the expression of one or more genes that positively regulate ROS production. ROS production may be reduced in the somatic cells during reprogramming by suppression of an endogenous target gene encoding a gene product that positively regulates ROS production using the target gene sequence in a number of ways generally known in the art, including, but not limited to, RNAi (siRNA, shRNA) techniques, microRNA, and CRISPR-Cas. Accordingly, the present technology provides a method for decreasing cellular ROS levels by suppressing a gene encoding a gene product that positively regulates ROS production, such as ST6GALNAC6, IGFBP5, PDGFD, SURF4, BOC, ADGRD1, MPDU1, RPS4Y1, MME, SET, DOK1, COLEC12, HOXC10, SULF2, ADAMTSL1, ELN, MGRN1, COL15A1, ZEB1, SFRP1, CLDN11, LGALS3BP, CHI3L1, SPG21, PI16, and MCFD2. Suppressing more than one genes encoding a gene product that positively regulates ROS production may further decrease ROS levels in a cell. In some embodiments, the one or more genes is targeted for down-regulation when the expression level is found to be at least 2-fold to 5-fold upregulated in a high ROS cell as compared to a low ROS cell. In some embodiments, the one or more genes is targeted for down-regulation when the expression level is found to be at least 5-fold upregulated in a high ROS cell as compared to a low ROS cell.

Methods for obtaining human somatic cells are well-known in the art, e.g., as described in Schantz and Ng (2004), A Manual for Primary Human Cell Culture, World Scientific Publishing Co., Pte, Ltd. In some embodiments, methods for obtaining somatic cells include obtaining a cellular sample, e.g., by a biopsy (e.g., a skin sample).

In some embodiments, the methods of the present technology relate to treating oocytes, including aged oocytes, with glutathione or derivatives thereof. In some embodiments, the treated oocytes may be used for in vitro fertilization (IVF) applications and may improve the success rate of IVF. In some embodiments, the treated oocytes increase the efficiency of in vitro embryo production and embryo quality. In some embodiments, the oocytes are selected for treatment with glutathione or derivatives thereof based on elevated ROS levels within the oocyte.

### I. Agents of the Present Technology

In some embodiments, the methods of the present technology comprise treating non-pluripotent cells with glutathione or derivatives thereof to produce iPSCs. In some embodiments, the glutathione is glutathione reduced ethyl ester, a stabilized form of glutathione.

In some embodiments, the methods of the present technology comprise treating non-pluripotent cells with L-Buthionine-sulfoximine (BSO) or derivatives thereof to generate iPSCs (FIG. 8).

### J. Modes of Administration and Effective Dosages

Any method known to those in the art for contacting a cell or tissue with the agents of the present technology may be employed. In some embodiments, the agent is glutathione or derivatives thereof. In some embodiments, the agent is glutathione reduced ethyl ester. In some embodiments the agent is L-Buthionine-sulfoximine (BSO) or derivatives thereof.

The dose and dosage regimen to be employed with respect to donor cell or tissue samples may depend on the level cellular ROS observed in a cell or tissue sample. The effective amount may be determined during pre-clinical trials and clinical trials by methods familiar to those skilled in the art. In some embodiments, the donor cell or tissue samples are contacted with 0.01 to 10 mM glutathione reduced ethyl ester. In some embodiments, the donor cell or tissue samples are contacted with 0.1 mM glutathione reduced ethyl ester. In some embodiments, the donor cell or tissue samples are contacted with 0.5 mM glutathione reduced ethyl ester. In some embodiments, the donor cell or tissue samples are contacted with 1 mM glutathione reduced ethyl ester. In some embodiments, the donor cell or tissue samples are contacted with 2 mM glutathione reduced ethyl ester. In some embodiments, the donor cell or tissue samples are contacted with 3 mM glutathione reduced ethyl ester. In some embodiments, the donor cell or tissue samples are contacted with 4 mM glutathione reduced ethyl ester. In some embodiments, the donor cell or tissue samples are contacted with 5 mM glutathione reduced ethyl ester. In some embodiments, the donor cell or tissue samples are contacted with 6 mM glutathione reduced ethyl ester. In some embodiments, the donor cell or tissue samples are contacted with 7 mM glutathione reduced ethyl ester. In some embodiments, the donor cell or tissue samples are contacted with 8 mM glutathione reduced ethyl ester. In some embodiments, the donor cell or tissue samples are contacted with 9 mM glutathione reduced ethyl ester. In some embodiments, the donor cell or tissue samples are contacted with 10 mM glutathione reduced ethyl ester or more.

In some embodiments, the donor cell or tissue samples are contacted with 0.01 to 10 mM L-Buthionine-sulfoximine (BSO). In some embodiments, the donor cell or tissue samples are contacted with 0.1 mM BSO. In some embodiments, the donor cell or tissue samples are contacted with 0.5 mM BSO. In some embodiments, the donor cell or tissue samples are contacted with 1 mM BSO. In some embodiments, the donor cell or tissue samples are contacted with 2 mM BSO. In some embodiments, the donor cell or tissue samples are contacted with 3 mM BSO. In some embodiments, the donor cell or tissue samples are contacted with 4 mM BSO. In some embodiments, the donor cell or tissue samples are contacted with 5 mM BSO. In some embodiments, the donor cell or tissue samples are contacted with 6 mM BSO. In some embodiments, the donor cell or tissue samples are contacted with 7 mM BSO. In some embodiments, the donor cell or tissue samples are contacted with 8 mM BSO. In some embodiments, the donor cell or tissue samples are contacted with 9 mM BSO. In some embodiments, the donor cell or tissue samples are contacted with 10 mM BSO or more.

Considering the day of transduction of reprogramming factors as day 0 (initiation of reprogramming), in some embodiments, the dosage regimen comprises treating the donor cells or tissue with glutathione from day-1 to 10. In some embodiments, the dosage regimen comprises treating the donor cells with glutathione from day-10 to 0, from day-9 to 0, from day-8 to 0, from day-7 to 0, from day-6 to 0, from day-5 to 0, from day-4 to 0, from day-3 to 0, from day-2 to 0, from day-1 to 0, from day 0 to 1, from day 0 to 2, from day 0 to 3, from day 0 to 4, from day 0 to 5, from day 0 to 6, from day 0 to 7, from day 0 to 8, from day 0 to 9, from day 0 to 10, or from any interval between days-10 to 10.

### K. Therapeutic Applications

The following discussion is presented by way of example only, and is not intended to be limiting.

In some embodiments, the iPSCs generated by the methods described herein have a variety of applications and therapeutic uses. In some embodiments, the methods disclosed herein are directed to the generation of iPSCs suitable for therapeutic applications, including transplantation into patients. In some embodiments, the methods of the present technology yield iPSCs that have a reduced oncogenic potential as they exhibit genomic stability and DNA damage repair signaling.

Because cancer stem cells (CSC; or tumor-initiating cells (TIC)) share mechanistic similarities with pluripotent stem cells, without wishing to be bound by theory, it is believed that a similar redox imbalance in TIC leads to the development of radiation/chemotherapy-resistant tumors. Accordingly, in some embodiments, the biomarkers described herein may predict higher tumorigenicity and the development of radiation/chemotherapy resistance in iPSC generated from somatic cell donors or TIC in individuals, thereby identifying those cells for treatment with glutathione or derivatives thereof.

### L. Kits

Also disclosed herein are kits for generating iPSCs from non-pluripotent cells. In some embodiments, the kits include glutathione reduced ethyl ester, reprogramming factors, and instructions for reprogramming a plurality of non-pluripotent cells, such as somatic cells derived from aged donors to generate A-iPSCs.

### EXPERIMENTAL EXAMPLES

The present technology is further illustrated by the following examples, which should not be construed as limiting in any way.

### Materials and Methods

Cell culture. ESC and iPSC were cultured in ESC media containing 10% FBS and 1,000 U/mL of LIP (ESGRO® Leukemia Inhibitory Factor [LIF], 1 million units/1 mL). For generation of ESC, established methods were used (see, e.g., Kim, et al. Nature 467:285-290 (2010)). For iPSC reprogramming of somatic cells, retrovirus expressing Oct4, Sox2, Klf4, and c-Myc were introduced. For the somatic cells containing inducible reprogramming factors, the media was supplemented with 2 μg/mL doxycycline (MP Biomedicals, doxycycline hyclate). For DNA and RNA isolation, ESC or iPSC were trypsinized and replated onto new tissue culture dishes for 30 min to remove feeder cells, and nucleic acids were extracted from the non-adherent cell suspension.

Generation of mouse Y-iPSC, mouse A-iPSC, human Y-iPSC, and human A-iPSC. Skin fibroblast cells (10⁶) were collected from B6CBA and B6129 mice, 5-day-old tail tip skin, and 1.4-year old tail tip skin; infected with retrovirus generated from pMX-mOCT4, pMX-SOX2, pMX-mKLF4,2, and pEYK-mMYC3 in 6-well dishes with 0.5 mL of each virul supernatant (total 2 mL per well; and spun at 2500 rpm at RT for 90 min (BenchTop Centrifuge, BeckmanCoulter, Allegra-6R). The cells were contacted with 3 mM glutathione reduced ethyl ester prior to and during the early stage of reprogramming (from one day before reprogramming virus infection to 10 days post reprogramming virus infection).

For the generation of human A-iPSC, 10⁵ skin fibroblasts such as young somatic cells (Y-SC) from MRCS and aged somatic cells (A-SC) from LS and AG4 donors aged 80 to 100 years were infected with retrovirus generated from the tetracistronic SFG-SV2 vector encoding for hOCT4, hSOX2, hKLF4, and hMYC in 6-well dishes with 0.5 mL of each viral supernatant (total 2 mL per well); and spun at 2500 rpm at RT for 90 min (BenchTop Centrifuge, BeckmanCoulter, Allegra-6R). The cells were contacted with 3 mM glutathione reduced ethyl ester prior to and during the early stage of reprogramming (from one day before reprogramming virus infection to 10 days post reprogramming virus infection).

Quantitative real time-PCT (Q-PCR) analysis. The expression levels of genes (ZSCAN10, GSS) were quantified by Q-PCR with Power SYBR Green PCR mastermix (Applied Biosystems). Total RNAs (1 μg) were reverse-transcribed in a volume of 20 μL using the M-MuLV Reverse Transcriptase system (New England Biolabs), and the resulting cDNA was diluted into a total volume of 200 μL. 10 μL of this synthesized cDNA solution was used for analysis. For pluripotent genes, each reaction was performed in a 25-μL volume using the Power SYBR Green PCR mastermix (Applied Biosystems). The conditions were programmed as follows: initial denaturation at 95° C. for 10 min followed by 40 cycles of 30 sec at 95° C., 1 min at 55° C., and 1 min at 72° C.; then 1 min at 95° C., 30 s at 55° C., and 30 sec at 95° C. All of the samples were duplicated, and the PCR reaction was performed using an Mx3005 reader (Stratagene), which can detect the amount of synthesized signals during each PCR cycle. The relative amounts of the mRNAs were determined using the MxPro program (Strategene). The amount of PCR product was normalized to the percentage of the expression level of β-actin.

Drug treatments. Phleomycin (Sigma) was added at 30 μg/mL for 2 hours. Cells were processed for analysis 30 min after phleomycin treatment unless indicated otherwise. After the 3-min recovery in ESC media, the cells were collected and processed for following experiments. For the detection of DNA damage response in the extended period, the cells were given 6 hours to recover after phleomycin treatment and were processed for H2AX immunostaining. In the DNA fragmentation assay, the cells were given 15 hours to recover. To check the mutatgenesis potential, the cells were treated with phleomycin 30 μg/mL for 2 hours and cultured for one passage after each treatment.

Immunoblot analysis. Treated and untreated cells (1×10⁵ cells) were collected 30 min after the 2-hour phleomycin treatment (30 μg/mL). To harvest protein 100-200 mL RIPA buffer (50 mM Tris-HCL [pH 7.4], 150 mM NaCl, 1% NP40, 0.25% Na-deoxycholate, 1 mM PMSF, protease inhibitor cocktail, and phosphatase inhibitor cocktail) was added to floating cell pellets and the remaining adherent cell. The samples were incubated on ice (10 min) and centrifuged (14,000 g, 10 min, 4° C.). Protein concentrations were determined using a BCA protein assay kit (Pierce). Samples were adjusted to the same concentration with RIPA buffer (3000 μg/mL) and were combined with Laemmli Sample Buffer (Biorad) and β-Mercaptoethanol (Sigma) then heated at 95° C. for 5 min and loaded onto a 4-15% Mini Protean TGX SDS-PAGE gel (BioRad). Samples on the SDS-PAGE gel were transferred to a 0.2-mm PVDF membrane at 100 V for 1 h, using a wet electro-transfer method (0.2 M glycine, 25 mM Tris, and 20% methanol). The membrane was blocked with 5% GSA in PBS-T (1 h at 4° C.), followed by incubation with primary antibodies anti-H2AX (Millipore, 05-636) (1:1000), anti-p53 (Leic Biosystems, P53-CMSP) (1:1000) anti-phospho-ATM (Pierce, MAI-2020) or anti-beta actin (Cell Signaling #4967) (1:5000) in blocking solution (5% BSA in phosphate-buffered saline containing Tween-20 [1:1000] PBS-T, overnight at 4° C.). After primary antibody incubation, membranes were washed three times in PBS-T prior to addition of secondary antibody labelled with peroxidase. Secondary antibodies were from Cell Signaling (1:10,000).

Copy number profiling analysis. Copy number profiling analysis was performed according to a published protocol (Baslan et al., Genome Research 25:1-11 (2015)).

Reactive oxygen species (ROS) analysis. The production of superoxide by mitochondria can be visualized in fluorescence microscopy using the MitoSOX™ Red reagent (M36008, Thermofisher scientific). MitoSOX™ Red reagent permeates live cells where it selectively targets mitochondria. It is rapidly oxidized by superoxide but not by other reactive oxygen species (ROS) and reactive nitrogen species (RNS). The oxidized product is highly fluorescent upon binding to nucleic acid. Fluorescence microscopy was used to visualize the fluorescence and imaging software (Image J) was used to quantify the staining of the different cell lines.

BSO treatment. BSO-A-iPSC were generated in the presence of 500 μM of L-Buthionine-sulfoximine (BSO, Sigma, B2515) starting on the end of reprogramming day 5. The treatment was kept throughout the end of reprogramming process and after picking the colonies.

MitoSOX RED Staining. MitoSOX Red staining (MitoSOX™ Red mitochondrial superoxide indicator *for live-cell Imaging, M36008, ThermoFisher Scientific, Waltham, Mass.) was performed according to Molecular Probes/Thermo Scientific protocol. Briefly, the MitoSOX™ reagent stock solution (5 mM, prepared in HBSS/Ca/Mg or suitable buffer) was diluted to make a 5 μM MitoSOX™ reagent working solution. 1.0-2.0 mL of the 5 μM MitoSOX™ reagent working solution was applied to cover cells adhering to coverslip(s). Cells were incubated for 10 minutes at 37° C., protected from light. Cells were washed before imaging.

Metabolomics Analysis. Metabolomics analysis was performed by the Southeast Center for Integrated Metabolomics (SECIM) at the University of Florida. Briefly, an untargeted liquid chromatography-mass spectrometry global metabolomics analysis was performed on human fibroblasts from aged donors (age between 80-100 years old) that are grouped as low and high ROS (reactive oxygen species). Eleven total samples, including High ROS (n=5) and Low ROS (n=6), were analyzed. All provided samples were extracted following standard cellular extraction procedure with pre-normalization to the sample protein content. Global metabolomics profiling was performed on a Thermo Q-Exactive Oribtrap mass spectrometer (ThermoFisher Scientific) with Dionex UHPLC (Dionex, Sunnyvale, Calif.) and autosampler. All samples were analyzed in both, positive and negative heated electrospray ionization, with a mass resolution of 35,000 at m/z 200 as separate injections. Separation was achieved on an ACE 18-pfp 100×2.1 mm, 2 μm column (Advanced Chromatography Technologies Ltd, Aberdeen, Scotland) with mobile phase A as 0.1% formic acid in water and mobile phase B as acetonitrile. The flow rate was 350 μL/min with a column temperature of 25° C. For ions analyzed in negative ion mode, 4 was injected onto the column, and for ions analyzed in positive ion mode, 2 μL was injected onto the column. Analysis data from positive and negative ion modes were separately subjected to statistical analyses. A total of 1,006 features were detected from the positive mode and 692 features were detected in the negative mode. All subsequent data analyses were normalized to the sum of metabolites for each sample. MZmine (freeware) was used to identify features, deisotope, align features and perform gap filling to fill in any features that may have been missed in the first alignment algorithm.

G4 and 8-oxoguanosine Immunostaining. Cells were fixed in 3.7% formaldehyde for 20 minutes at room temperature and washed with phosphate buffered saline (PBS). Samples were then permeabilized with 0.1 Triton X-100 in PBS for 20 minutes and blocked for 1 hour with 3% bovine serum albumin (BSA) in PBS-T, followed by incubation with primary antibodies for 2 hours at room temperature or overnight at 4° C. Anti-DNA G-quadruplex (G4) Antibody, clone 1H6 (MABE1126, EMD Millipore, Burlington, Mass.) was used for the detection of DNA G-quadruplex and ANTI-8 HYDROXYGUANOSINE AB (N45.1) AB48508 from Abcam (Cambridge, United Kingdom). Primary antibodies were used at a 1:250 dilution and 1:50 respectively. Alexa 568-conjugated goat anti-mouse IgM (A-21124) was from Molecular Probes (Eugene, Oreg.). Secondary antibodies were used at a 1:1000 dilution. The nuclei were stained with 4′,6-diamidino-2-phenylindole (DAPI, Sigma, St. Louis, Mo.). Image quantification was performed with Image J (NIH, Bethesda, Md.), quantifying mean fluorescence intensity in the nuclear regions.

GSH and BSO Treatment, Time-Course Experiment. High ROS fibroblasts were treated by adding glutathione reduced ethyl ester (3 mM, GEE; Gold Biotechnology Inc., St. Louis, Mo.) to the media. Treated High ROS fibroblasts were then fixed and processed for staining at multiple timepoints. Low ROS fibroblasts were treated with L-Buthionine-sulfoximine (500 μM, BSO; Sigma, St. Louis, Mo.) to the media. Treated Low ROS fibroblasts were then fixed and processed for staining at multiple timepoints.

CHIP-SEQ for G4 Structures. Chromatin immunoprecipitation (ChIP) was performed according to the published protocol (see Carey, M. F., et al., Chromatin Immunoprecipitation (ChIP), COLD SPRING HARB PROTOC (CSH Press) (2009)). Anti-DNA G-quadruplex (G4) Antibody, clone 1H6 (MABE1126, EMD Millipore, Burlington, Mass.) was used for the detection of DNA G-quadruplex.

Lentivirus Production. 293T cells were seeded overnight at 5×10⁶ cells per 150-mm dish with DMEM supplemented with 10% FBS and penicillin/streptomycin. The cells were transfected with 1.0× SMARTvector Inducible Human CHI3L1 hEF1a-TurboRFP shRNA (Dharmacon Inc., Lafayette, Colo.) with calcium phosphate cell transfection (CalPhos™ Mammalian Transfection Kit, Takara Bio Inc., Kusata Shiga Prefecture, Japan). Forty-eight hours after transfection, the medium containing the lentivirus was collected and the cellular debris was removed with centrifugation. The supernatant was filtered through a 0.45-μm filter, and the lentivirus was pelleted with ultracentrifugation at 33,000 rpm in a 45Ti rotor (Beckman Coulter, Pasadena, Calif.) for 90 min at 4° C. The lentivirus particles were re-suspended in DMEM medium and stored at −80° C.

Quantitative Real Time PCR (Q-PCR) Analysis. The expression levels of genes were quantified by Q-PCR. Total RNA (1 μg) was reverse transcribed in a volume of 20 μL using the M-MuLV Reverse Transcriptase system (New England Biolabs, Ipswich, Mass.), and the resulting cDNA was diluted into a total volume of 200 μL. 10 μL of this synthesized cDNA solution was used for analysis. Each reaction was performed in a 25 μL volume using the Power SYBR Green PCR Mastermix (Applied Biosystems, Foster City, Calif.). The conditions were programmed as follows: initial denaturation at 95° C. for 10 min followed by 40 cycles of 30 sec at 95° C., 1 min at 55° C., 1 min at 72° C., 1 min at 95° C., 30 s at 55° C., and 30 sec at 95° C. All of the samples were duplicated, and the PCR reaction was performed using a Mx3005P reader (Stratagene, San Diego, Calif.), which can detect the amount of synthesized signals during each PCR cycle. The relative amounts of the mRNAs were determined using the MxPro program (Stratagene). The amount of PCR product was normalized to a percentage of the expression level of GAPDH. The PCR products were also evaluated on 1.2% agarose gels after staining with ethidium bromide. The primers used to amplify the cDNA were the following:

hGAPDH:
(SEQ ID NO: 1)
CACCGTCAAGGCTGAGAACG
and
(SEQ ID NO: 2)
GCCCCACTTGATTTTGGAGG
hZSCAN10:
(SEQ ID NO: 3)
CCTTACTCTCAGGAGCGCAG
and
(SEQ ID NO: 4)
TGTGCCAGAGAATAAGGCGT
hOCT4:
(SEQ ID NO: 5)
CCTCACTTCACTGCACTGTA
and
(SEQ ID NO: 6)
CAGGTTTTCTTTCCCTAGCT
hSOX2:
(SEQ ID NO: 7)
CCCAGCAGACTTCACATGT
and
(SEQ ID NO: 8)
CCTCCCATTTCCCTCGTTTT
hMYC:
(SEQ ID NO: 9)
TGCCTCAAATTGGACTTTGG
and
(SEQ ID NO: 10)
GATTGAAATTCTGTGTAACTGC
hKLF4:
(SEQ ID NO: 11)
GATGAACTGACCAGGCACTA
and
(SEQ ID NO: 12)
GTGGGTCATATCCACTGTCT
hLIN28A:
(SEQ ID NO: 13)
CGGCCAAAAGGAAAGAGCAT
and
(SEQ ID NO: 14)
GCTTGCATTCCTTGGCATGAT
LIN28B:
(SEQ ID NO: 15)
GAGAGGGAAGCCCCTTGGAT
and
(SEQ ID NO: 16)
ACTGGTTCTCCTTCTTTTAGGCT
hNanog:
(SEQ ID NO: 17)
CAGCCCCGATTCTTCCACCAGTCCC
and
(SEQ ID NO: 18)
CGGAAGATTCCCAGTCGGGTTCACC

PDS Treatment. The G-quadruplex DNA stabilizing drug pyridostatin (Apex Biotechnology Co., Taiwan) was applied at a working concentration 10 μM in the media for the time indicated in every experiment.

### Example 1: Glutathione Recovers Genomic Stability in A-iPSC

Cellular ROS levels (detected by MitoSOX staining) were low in the young donor somatic cells (Y-SC) from B6CBA mouse and the aged donor somatic cells (A-SC) from B6129 mouse, but high in the A-SC from B6CBA mouse (FIG. 7A). This variability among the different tissue donors was also observed in human somatic cells in low cellular ROS levels in the Y-SC from MRCS donor and the A-SC from LS donor, but high in the A-SC from AG4 donor (FIG. 7B). Quantification of cellular ROS (FIGS. 7C, 7D) confirmed the variability of the cellular ROS levels from the various donor somatic cells in mouse and human. Interestingly, the cellular ROS level among the different tissue donors is highly correlated with a poor DNA damage response/genomic instability in the reprogrammed iPSC, generated with the Y-SC from B6CBA mouse/A-SC from B6129 mouse vs. the A-SC from B6CBA mouse (FIG. 7E). The human Y-SC from MRCS donor/human A-SC from LS donor vs. the human A-SC from AG4 donor also shows the strong relationship between cellular ROS levels and a poor DNA damage response/genomic instability in the reprogrammed iPSC (FIG. 7F).

To test the direct effect of the cellular ROS reduction on the recovery of DNA damage response/genomic stability in the reprogrammed A-iPSC, the stabilized form of glutathione chemical (glutathione reduced ethyl ester) was used to reduce the cellular ROS levels. Glutathione reduced ethyl ester treatment in the A-SC from AG4 donor reduces the cellular ROS level (FIG. 7B, 7D).

The effect of glutathione reduced ethyl ester treatment on human AG4 fibroblast clones, which show a higher level of MitoSOX staining, prior to and during the first 10 days of A-hiPSC reprogramming was examined. The results indicate that treatment protects the DNA damage response (FIG. 7G) and maintains genomic stability (upper panel, FIG. 7H) compared to untreated A-hiPSC (lower panel, FIG. 7H) in significant statistical difference (p=0.00005). In addition, ZSCAN10 levels in the A-iPSC were elevated with glutathione reduced ethyl ester treatment (FIG. 7I), indicating that glutathione treatment also influences the epigenetic changes and pluripotent gene expression during iPSC reprogramming. GSS levels in the A-iPSC were reduced with glutathione reduced ethyl ester treatment (FIG. 7J).

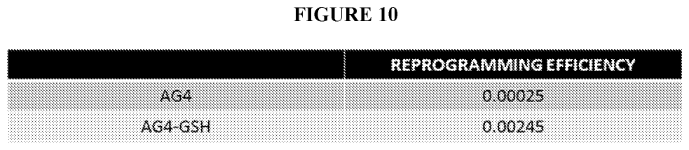

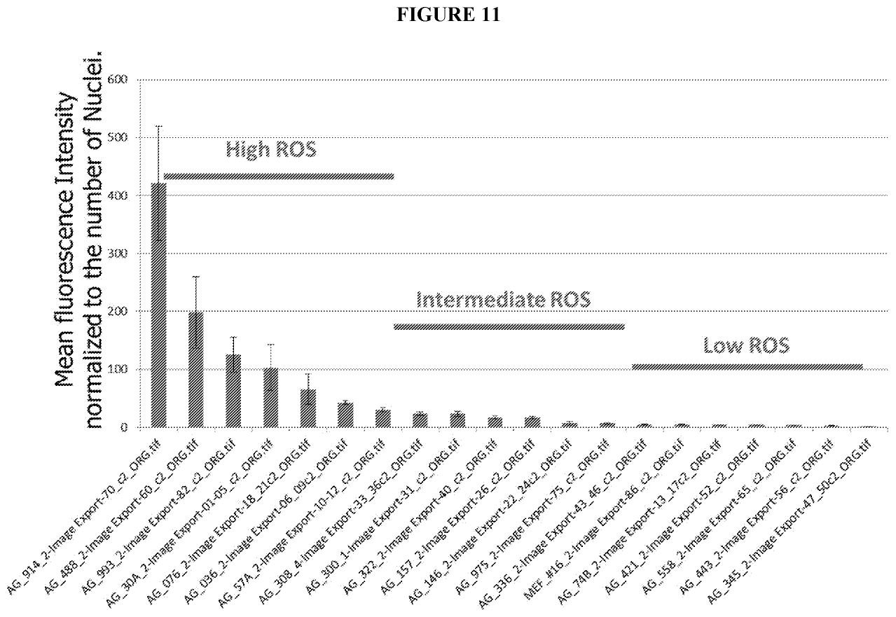

The effect of glutathione reduced ethyl ester treatment on the reprogramming efficiency of human AG4 fibroblasts was also examined. A control group of AG4 fibroblast (AG4) cells and a treatment group of glutathione reduced ethyl ester-AG4 fibroblast (AG4-GSH) cells (100,000 cells in each group) were cultured and reprogrammed according to the methods described herein. At day 21, iPSC colonies were counted and the reprogramming efficiency was determined by the ratio between the number of donor cells receiving the full set of reprogramming factors and the number of reprogrammed colonies generated. The results shown in FIG. 10 demonstrate that treatment of aged somatic cells with glutathione reduced ethyl ester increased reprogramming efficiency by approximately 10-fold.

Accordingly, these results show that treatment of aged somatic with glutathione reduced ethyl ester prior to initiation of reprogramming, during reprogramming, and/or after reprogramming produces A-iPSCs with increased genomic stability, increased DNA damage response, increased reprogramming efficiency, increased ZSCAN10 expression levels, and reduced GSS expression levels as compared to that observed in A-iPSCs produced from untreated control aged somatic cells grown under similar conditions and/or comparable to that observed in young iPSCs (Y-iPSCs) or ESCs.

### Example 2: BSO Recovers DNA Damage Response in A-iPSC

BSO-A-iPSC were generated in the presence of 500 μM of L-Buthionine-sulfoximine (BSO, Sigma B2515) starting on the end of reprogramming day 5 as previously described (see, e.g., Ji, et al. Experimental & Molecular Medicine 42:175-186 (2010)). Immunoblot of pATM shows recovery of the DNA damage response after phleomycin treatment in ten independent clones of A-iPSC with BSO (0.5 mM)-mediated inhibition of GSS (FIG. 8). Accordingly, these results show that treatment of aged somatic cells with BSO prior to initiation of reprogramming, during reprogramming, and/or after reprogramming produces A-iPSCs with increased DNA damage response as compared to that observed in A-iPSCs produced from untreated control aged somatic cells grown under similar conditions and/or comparable to that observed in ESCs.

### Example 3: Epigenetic and Expression Regulatory Landscape in Somatic Cells with High and Low ROS by RNA-Seq

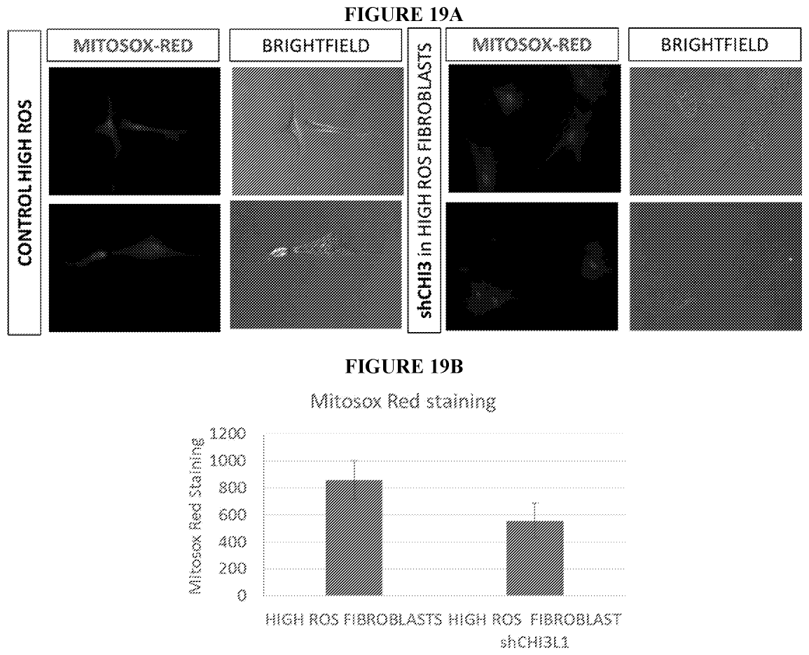

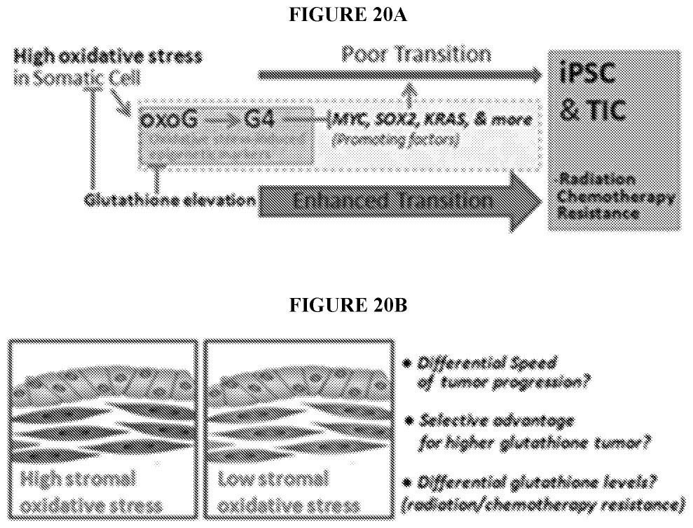

Gene expression analysis revealed differentially regulated genes that represent both upstream and downstream targets of ROS regulatory pathways. Optimal conditions to modify cellular ROS levels using GEE (3 mM 4 hours, decreases ROS/G4), BSO (0.5 mM 4 hours, increases ROS/G4), and pyridostatin (PDS: G4 structure stabilizer, 10 μM 4 hours, increases G4) have also been developed. These tools have been used to modulate ROS/G4 levels in somatic cells. The treatment of high-ROS somatic cells with GEE or low-ROS somatic cells with BSO will target the pathways downstream of ROS. Regulatory pathways downstream were then differentiated from upstream regulatory pathways by comparing the differentially regulated genes between high- and low-ROS somatic cells without GEE or BSO treatment (i.e., subtraction of the downstream targets regulated by GEE and BSO treatment from all differentially regulated genes between the somatic cells with high and low ROS will identify the potential upstream targets of ROS regulatory pathways). Two high-ROS and two low-ROS donor somatic cells were treated with GEE/BSO and analyzed. Among the differentially expressed genes (more than 5-fold) between high/low ROS fibroblasts (102 genes), the downstream targets of ROS were defined (75 differentially expressed genes with GEE/BSO treatment), and the potential genes upstream of ROS (27 genes that were not influenced by GEE/BSO treatment, FIG. 18). Since the regulatory genes upstream of ROS such as PI16, CHI3L1, and ZEB1 have been reported to be involved in ROS generation, the significant reduction of ROS by shRNA infection has been observed (FIG. 19) as valuable targets to overcome ROS-induced aging mechanism as a cancer prevention tool.

### Example 4: Combinatorial Approach for Identifying ROS Levels in Donor Somatic Cells

Non-pluripotent donor cells (e.g., somatic cells) which are selected for treatment by the methods of the present technology will be analyzed by a series of techniques to identify oxidative-stress related biomarkers in the donor cells which may predict decreased reprogramming efficiency, elevated tumorigenicity, and/or the development of radiation/chemotherapy resistance in the iPSCs generated from somatic cell donors. Somatic donor cells which may be identified to have elevated oxidative-stress profiles may be selected for treatment with glutathione or a derivative thereof. Somatic donor cells will undergo metabolomic profiling analyses to determine the cellular ROS levels based on the metabolic profile signature. Additionally or alternatively, following metabolic analysis, non-pluripotent donor cells (e.g., somatic cells) will be analyzed using RNA-Seq to determine gene expression profiles both upstream and downstream of regulatory ROS pathways. Somatic donor cells which may be identified to have elevated gene expression profiles upstream of ROS, may be selected for treatment with glutathione or a derivative thereof. Additionally or alternatively, nuclear oxidative stress will be analyzed using known immunohistochemical analyses. Briefly, non-pluripotent donor cells (e.g., somatic cells) will be stained with an 8-oxo-guanosine antibody, which binds to oxidized DNA in the nucleus due to high levels of ROS. Following staining of donor cells, cells will be imaged and compared to control cells to determine relative levels of ROS. Somatic donor cells which may be identified to have elevated levels of ROS, may be selected for treatment with glutathione or a derivative thereof. Additionally or alternatively, non-pluripotent donor cells (e.g., somatic cells) will also be analyzed for endogenous levels of G4 DNA levels using ChIP-Seq with an anti-G4 antibody. As shown in FIG. 15, donor cells with elevated levels of ROS should have elevated levels of G4 DNA. Somatic donor cells which may be identified to have elevated levels of ROS may be selected for treatment with glutathione or a derivative thereof.

### Example 5: Metabolic Profiling Analysis Reveals Distinct ROS-Associated Profile

The results of the metabolic profiling analyses shown in FIGS. 12A and 12B demonstrate that cells (e.g., somatic fibroblasts) derived from aged donors have distinct metabolic profiles based on cellular ROS levels. The results of the metabolic profiling analysis comparing the metabolome from high ROS donor cells (n=5) to low ROS donor cells (n=6) revealed 41 significantly altered metabolites, four of which have been characterized. The results of the profiling analysis are further summarized in Tables 1 and 2. The fold-change was calculated by dividing the average level of each metabolite identified in the metabolome of the high ROS donor cells (n=5) from the average level of each metabolite identified in the metabolome of the low ROS donor cells (n=6). The raw mass spectrometry data for the characterized metabolites are provided in Tables 3 and 4. In total, as described above, a total of 1006 features were detected for the positive ion mode and 692 features were detected in the negative ion mode. Identified but uncharacterized features from the high ROS and low ROS donor cells can be identified by searching against know libraries, filtering for metabolites, and running standards to confirm the identification of the metabolite. Donor somatic cells exhibiting a metabolic profile similar to that of high ROS control somatic cells may be selected for glutathione treatment. It is anticipated that high ROS donor somatic cells exhibiting the metabolic profile similar to that of high ROS control somatic cells selected for treatment with glutathione or derivatives thereof will generate iPSCs characterized by one or more of increased genomic stability, increased DNA damage response, increased ZSCAN10 expression, and reduced glutathione synthetase (GSS) expression compared to iPSCs produced from untreated control somatic cells grown under similar conditions. Accordingly, these results will demonstrate that the metabolic profile of donor somatic cells may serve as a biomarker for elevated cellular ROS levels identifying the cells for treatment before, during, and/or after iPSC reprogramming.

TABLE 1
Number of significant metabolites with p value < 0.05
analyzed by Volcano Plot.

| | Significant metabolites (p-value < 0.05) | | |
|---|---|---|---|
| | Identified | Unknown | Total |
| Data Set | Metabolites | Metbolites | metabolites |
| Positive Mode | 2 | 26 | 28 |
| Negative Mode | 2 | 11 | 13 |

TABLE 2
Significant known metabolites from Volcano Plot
in the positive and negative mode data sets.

| | FC | p. value |
|---|---|---|
| Metabolite (Positive Mode) | | |
| ADENOSINE_268.1033-5.62 | 0.42783 | 0.077977 |
| CYTIDINE_244.0923-1.58 | 0.1487 | 0.092661 |
| Metabolite (Negative Mode) | | |
| XANTHINE_151.0261-2.41 | 2.2644 | 0.033862 |
| 3′-CMP_322.0445-1.48 | 2.799 | 0.041436 |

TABLE 3
Mass Spectrometry Data for Metabolomics Analysis (positive
ion mode)

| | average | average | |
|---|---|---|---|
| | for high | for low | |
| | ROS cell | ROS cell | fold |
| Name | lines 1-5 | lines 6-11 | difference |
| L-KYNURENINE | 2.00E+05 | 4.43E+04 | 4.52E+00 |
| CREATININE | 2.50E+06 | 8.80E+05 | 2.84E+00 |
| CITRULLINE | 6.40E+05 | 2.81E+05 | 2.28E+00 |
| L-CYSTATHIONINE | 1.40E+05 | 6.55E+04 | 2.13E+00 |
| PUTRESCINE | 2.57E+05 | 1.46E+05 | 1.76E+00 |
| Isovalerylcarnitine | 4.41E+05 | 2.72E+05 | 1.62E+00 |
| SEROTONIN-NH3 | 3.74E+04 | 2.32E+04 | 1.61E+00 |
| L-Cystine | 4.98E+05 | 3.12E+05 | 1.60E+00 |
| Isobutyrylcarnitine | 9.27E+04 | 6.00E+04 | 1.54E+00 |
| L-LYSINE | 4.76E+05 | 3.09E+05 | 1.54E+00 |
| L-HISTIDINE | 1.68E+07 | 1.09E+07 | 1.54E+00 |
| N-BOC-L-Tryptophan | 7.05E+05 | 4.63E+05 | 1.52E+00 |
| Acyl-Carnitine (5-OH) | 5.04E+04 | 3.37E+04 | 1.49E+00 |
| GLUCOSE/FRUCTOSE- | 3.77E+05 | 2.53E+05 | 1.49E+00 |
| PHOSPHATE | | | |
| L-ASPARAGINE | 1.31E+07 | 9.21E+06 | 1.42E+00 |
| 5-OXO-L-PROLINE | 2.15E+06 | 1.52E+06 | 1.42E+00 |
| N-ALPHA-ACETYL-L-LYSINE | 1.22E+05 | 8.67E+04 | 1.41E+00 |
| ALPHA-AMINOADIPATE/N- | 5.44E+05 | 4.01E+05 | 1.36E+00 |
| METHYL-L-GLUTAMATE | | | |
| L-Isoleucine | 3.30E+07 | 2.45E+07 | 1.35E+00 |
| GLUCOSAMINE/MANNOSAMINE | 3.38E+05 | 2.51E+05 | 1.34E+00 |
| N(PAI)-METHYL-L-HISTIDINE | 6.32E+05 | 4.77E+05 | 1.32E+00 |
| ALDO/KETO-HEXOSE | 2.90E+06 | 2.22E+06 | 1.31E+00 |
| L-LYSINE | 1.38E+07 | 1.06E+07 | 1.30E+00 |
| Phenylalanine | 5.78E+07 | 4.51E+07 | 1.28E+00 |
| Methionine Sulfoxide | 1.09E+07 | 8.49E+06 | 1.28E+00 |
| Tryptophan | 9.22E+06 | 7.31E+06 | 1.26E+00 |
| GLYCINE | 2.04E+06 | 1.63E+06 | 1.26E+00 |
| L-LEUCINE | 1.08E+08 | 8.59E+07 | 1.26E+00 |
| Tryptophan-NH3 | 5.62E+06 | 4.48E+06 | 1.25E+00 |
| HEXOSE-6-PHOSPHATE | 2.82E+05 | 2.27E+05 | 1.24E+00 |
| 2-hydroxyglutarate-water | 5.89E+04 | 4.76E+04 | 1.24E+00 |
| L-METHIONINE | 2.04E+07 | 1.65E+07 | 1.24E+00 |
| N-Methylnicotinamide | 3.50E+07 | 2.84E+07 | 1.23E+00 |
| L-GLUTAMINE | 9.98E+07 | 8.25E+07 | 1.21E+00 |
| ALANINE/SARCOSINE | 1.04E+07 | 8.63E+06 | 1.20E+00 |
| BOC-L-Tyrosine | 3.85E+05 | 3.23E+05 | 1.19E+00 |
| N-ACETYL-HEXOSAMINE | 2.36E+05 | 1.99E+05 | 1.19E+00 |
| THREONINE/HOMOSERINE | 3.84E+07 | 3.24E+07 | 1.18E+00 |
| SPERMIDINE | 3.56E+07 | 3.03E+07 | 1.18E+00 |
| N-BOC-L-Tryptophan | 1.63E+07 | 1.40E+07 | 1.17E+00 |
| L-SERINE | 3.30E+07 | 2.83E+07 | 1.17E+00 |
| N(PAI)-METHYL-L-HISTIDINE | 6.90E+05 | 5.98E+05 | 1.15E+00 |
| L-CYSTEINE | 1.22E+06 | 1.07E+06 | 1.14E+00 |
| L-PROLINE | 1.11E+08 | 9.87E+07 | 1.13E+00 |
| DEOXYCARNITINE | 5.86E+05 | 5.23E+05 | 1.12E+00 |
| 2-Hydroxyphenylalanine | 2.48E+07 | 2.22E+07 | 1.12E+00 |
| 5-HYDROXY-L-TRYPTOPHAN | 1.00E+04 | 9.00E+03 | 1.11E+00 |
| 1,2-Benzisothiazolin-3-one | 5.89E+04 | 5.39E+04 | 1.09E+00 |
| N-Acetyl-Arginine | 8.04E+03 | 7.41E+03 | 1.08E+00 |
| Caffeine-D3 | 7.24E+06 | 6.78E+06 | 1.07E+00 |
| 5-HYDROXY-L-TRYPTOPHAN | 1.15E+04 | 1.07E+04 | 1.07E+00 |
| N-Butylbenzenesulfonamide | 2.06E+05 | 1.95E+05 | 1.06E+00 |
| PYRIDOXAL | 3.38E+05 | 3.20E+05 | 1.06E+00 |
| N-BOC-L-tert-Leucine | 2.71E+06 | 2.57E+06 | 1.06E+00 |
| L-ARGININE | 2.02E+08 | 1.92E+08 | 1.05E+00 |
| Creatine-D3 | 2.52E+07 | 2.40E+07 | 1.05E+00 |
| ORNITHINE | 5.26E+05 | 5.01E+05 | 1.05E+00 |
| BOC-D-Phenylalanine | 2.59E+07 | 2.50E+07 | 1.04E+00 |
| Orthophosphate | 5.98E+06 | 5.76E+06 | 1.04E+00 |
| CYTOSINE | 7.52E+05 | 7.33E+05 | 1.03E+00 |
| L-Leucine-D10 | 1.01E+07 | 9.91E+06 | 1.02E+00 |
| Tryptophan-2,3,3-D3 | 2.66E+07 | 2.60E+07 | 1.02E+00 |
| L-Tyrosine 13C6 | 1.49E+06 | 1.46E+06 | 1.02E+00 |
| Butytylcarnitine | 1.22E+05 | 1.20E+05 | 1.02E+00 |
| Tryptophan-2,3,3-D3-NH3 | 1.50E+07 | 1.48E+07 | 1.01E+00 |
| N-BOC-L-Aspartic Acid | 2.04E+06 | 2.01E+06 | 1.01E+00 |
| N-ACETYLPUTRESCINE | 5.51E+06 | 5.52E+06 | 9.98E−01 |
| BOC-L-Tyrosine | 4.67E+07 | 4.68E+07 | 9.98E−01 |
| L-Leucine 13C6 | 9.74E+06 | 9.79E+06 | 9.95E−01 |
| DL-5-HYDROXYLYSINE | 1.12E+05 | 1.14E+05 | 9.88E−01 |
| 5-AMINOLEVULINIC ACID | 6.42E+05 | 6.54E+05 | 9.81E−01 |
| HYPDXANTHINE | 1.64E+07 | 1.68E+07 | 9.78E−01 |
| Pantothenic Acid | 3.70E+05 | 3.80E+05 | 9.75E−01 |
| ASPARTATE | 8.05E+06 | 8.48E+06 | 9.49E−01 |
| HEXOSE-6-PHOSPHATE | 2.90E+04 | 3.06E+04 | 9.49E−01 |
| N-METHYL-D-ASPARTIC ACID | 1.01E+08 | 1.08E+08 | 9.37E−01 |
| GUANINE | 3.28E+06 | 3.58E+06 | 9.17E−01 |
| Ribulose 5-Phosphate | 1.30E+05 | 1.42E+05 | 9.12E−01 |
| N-ACETYLNEURAMINATE | 3.57E+05 | 3.97E+05 | 8.98E−01 |
| Diphenylamine | 9.35E+04 | 1.04E+05 | 8.95E−01 |
| Allopurinol | 1.73E+07 | 1.94E+07 | 8.89E−01 |
| URIDINE | 5.58E+05 | 6.35E+05 | 8.79E−01 |
| Leu Pro | 1.12E+06 | 1.29E+06 | 8.74E−01 |
| CARNOSINE | 1.04E+06 | 1.20E+06 | 8.63E−01 |
| ETHYLMALONIC ACID | 4.76E+04 | 5.66E+04 | 8.42E−01 |
| NICOTINAMIDE | 3.32E+06 | 3.95E+06 | 8.40E−01 |
| Leu Pro | 2.12E+05 | 2.65E+05 | 7.99E−01 |
| Ribulose 5-Phosphate | 1.15E+05 | 1.45E+05 | 7.94E−01 |
| PHOSPHOCHOLINE | 2.86E+07 | 3.68E+07 | 7.78E−01 |
| CREATINE | 2.00E+07 | 2.62E+07 | 7.63E−01 |
| L-CARNITINE | 1.09E+07 | 1.55E+07 | 7.02E−01 |
| TAURINE | 5.58E+06 | 7.99E+06 | 6.99E−01 |
| METHYL BETA-D- | 2.83E+06 | 4.51E+06 | 6.26E−01 |
| GALACTOSIDE | | | |
| LL-2,6- | 2.29E+05 | 3.70E+05 | 6.18E−01 |
| DIAMINOHEPTANEDIOATE | | | |
| D-Ribose | 1.30E+05 | 2.14E+05 | 6.11E−01 |
| Glycerophosphocholine | 7.75E+06 | 1.33E+07 | 5.84E−01 |
| ADENOSINE | 8.15E+04 | 1.41E+05 | 5.79E−01 |
| Choline | 1.53E+07 | 2.66E+07 | 5.75E−01 |
| Glutathione Disulfide | 3.60E+05 | 6.77E+05 | 5.32E−01 |
| L-2-PHOSPHOGLYCERIC ACID | 7.61E+04 | 1.65E+05 | 4.62E−01 |
| CYTIDINE | 8.99E+04 | 5.65E+05 | 1.59E−01 |

TABLE 4
Mass Spectrometry Data for Metabolomics Analysis
(negative ion mode)

| | average | average | |
|---|---|---|---|
| | for high | for low | |
| | ROS cell | ROS cell | fold |
| Name | lines 1-5 | lines 6-11 | difference |
| 3′-CMP | 1.05E+05 | 3.61E+04 | 2.90E+00 |
| XANTHINE | 1.67E+05 | 7.37E+04 | 2.27E+00 |
| 6-PHOSPHOGLUCONIC ACID | 9.97E+05 | 5.66E+05 | 1.76E+00 |
| L-HISTIDINE | 7.94E+06 | 5.13E+06 | 1.55E+00 |
| C6H12O6- | 3.53E+05 | 2.29E+05 | 1.54E+00 |
| HEXOSE/KETOSE/INOSITOL | | | |
| N-METHYL-L-GLUTAMATE | 2.32E+05 | 1.63E+05 | 1.42E+00 |
| L-VALINE | 8.25E+04 | 5.84E+04 | 1.41E+00 |
| LEUCINE | 9.36E+04 | 6.84E+04 | 1.37E+00 |
| L-CYSTEIC ACID | 2.68E+05 | 2.03E+05 | 1.32E+00 |
| ASPARAGINE | 3.68E+06 | 2.83E+06 | 1.30E+00 |
| 3-SULFINO-L-ALANINE | 1.47E+05 | 1.13E+05 | 1.30E+00 |
| N-ACETYL-D-MANNOSAMINE | 2.58E+05 | 2.00E+05 | 1.29E+00 |
| L-METHIONINE | 5.70E+05 | 4.69E+05 | 1.22E+00 |
| Tryptophan | 4.24E+05 | 3.51E+05 | 1.21E+00 |
| L-CYSTATHIONINE | 3.45E+04 | 2.92E+04 | 1.18E+00 |
| L-ISOLEUCINE | 3.00E+04 | 2.58E+04 | 1.16E+00 |
| L-SERINE | 9.36E+06 | 8.18E+06 | 1.14E+00 |
| 6-keto prostaglandin 1? | 3.00E+06 | 2.63E+06 | 1.14E+00 |
| DIHYDROXYACETONE | 7.22E+05 | 6.40E+05 | 1.13E+00 |
| PHOSPHATE | | | |
| D-GLUCURONIC ACID/D- | 1.46E+04 | 1.32E+04 | 1.11E+00 |
| GLUCURONOLACTONE/D-(+)- | | | |
| GALACTURONIC ACID | | | |
| N-BOC-L-Tryptophan | 3.99E+07 | 3.63E+07 | 1.10E+00 |
| N-BOC-L-Tryptophan | 2.44E+07 | 2.22E+07 | 1.10E+00 |
| L-TYROSINE | 6.13E+05 | 5.69E+05 | 1.08E+00 |
| GLUCOSE/FRUCTOSE | 1.05E+06 | 9.78E+05 | 1.07E+00 |
| N-BOC-L-Aspartic Acid | 4.42E+06 | 4.18E+06 | 1.06E+00 |
| L-GLUTAMINE | 8.26E+06 | 7.88E+06 | 1.05E+00 |
| GLUCOSE/FRUCTOSE | 3.15E+06 | 3.01E+06 | 1.05E+00 |
| GLYCOLATE | 4.69E+05 | 4.51E+05 | 1.04E+00 |
| N-BOC-L-tert-Leucine | 3.37E+07 | 3.25E+07 | 1.04E+00 |
| PYRIDOXAL | 3.38E+04 | 3.28E+04 | 1.03E+00 |
| N-BOC-L-Aspartic Acid | 1.64E+07 | 1.59E+07 | 1.03E+00 |
| 4-Hydroxyphenylacetate | 3.48E+04 | 3.40E+04 | 1.02E+00 |
| THREONINE/HOMOSERINE | 1.31E+06 | 1.28E+06 | 1.02E+00 |
| N-BOC-L-tert-Leucine | 9.39E+06 | 9.23E+06 | 1.02E+00 |
| MALATE | 2.73E+06 | 2.70E+06 | 1.01E+00 |
| Succinic-2,2,3,3-D4 Acid | 3.75E+06 | 3.71E+06 | 1.01E+00 |
| 2′,4′- | 8.71E+04 | 8.68E+04 | 1.00E+00 |
| DIHYDROXYACETOPHENONE | | | |
| 6-Hydroxycaproic acid | 1.81E+05 | 1.80E+05 | 1.00E+00 |
| BOC-L-Tyrosine | 1.26E+08 | 1.26E+08 | 1.00E+00 |
| BOC-L-Tyrosine | 5.44E+07 | 5.50E+07 | 9.91E−01 |
| Tryptophan-2,3,3-D3 | 1.09E+06 | 1.11E+06 | 9.84E−01 |
| Creatine-D3 | 5.97E+05 | 6.07E+05 | 9.83E−01 |
| HYPDXANTHINE | 2.94E+06 | 3.00E+06 | 9.81E−01 |
| Salicylic Acid-D4 | 1.20E+06 | 1.22E+06 | 9.81E−01 |
| D-RIBOSE 5- | 2.24E+06 | 2.37E+06 | 9.47E−01 |
| PHOSPHATE/RIBULOSE 5- | | | |
| PHOSPHATE | | | |
| 2-hydroxyglutarate | 1.14E+05 | 1.21E+05 | 9.44E−01 |
| ASPARTATE | 1.02E+07 | 1.08E+07 | 9.43E−01 |
| N-ACETYL-DL-GLUTAMIC | 3.00E+04 | 3.24E+04 | 9.26E−01 |
| ACID | | | |
| S-CARBOXYMETHYL-L- | 1.49E+05 | 1.61E+05 | 9.25E−01 |
| CYSTEINE | | | |
| L-ARGININE | 4.60E+06 | 5.07E+06 | 9.07E−01 |
| URIDINE | 3.56E+06 | 3.94E+06 | 9.01E−01 |
| LACTATE-dimer | 3.57E+05 | 3.98E+05 | 8.96E−01 |
| Pantothenic Acid | 3.92E+05 | 4.38E+05 | 8.95E−01 |
| Guanosine | 2.58E+06 | 2.88E+06 | 8.94E−01 |
| N-ACETYLNEURAMINATE | 9.88E+05 | 1.11E+06 | 8.89E−01 |
| CMP | 1.36E+05 | 1.55E+05 | 8.77E−01 |
| L-GLUTAMIC ACID | 3.18E+07 | 3.64E+07 | 8.72E−01 |
| GUANINE | 3.96E+05 | 4.69E+05 | 8.44E−01 |
| SUCCINATE | 2.47E+06 | 3.19E+06 | 7.75E−01 |
| D-GLUCOSAMINE 6- | 5.62E+05 | 7.54E+05 | 7.45E−01 |
| PHOSPHATE | | | |
| Nicotinamide ribotide | 1.18E+05 | 1.61E+05 | 7.33E−01 |
| TAURINE | 8.37E+06 | 1.14E+07 | 7.31E−01 |
| GLYCERIC ACID | 7.95E+05 | 1.09E+06 | 7.31E−01 |
| SARCOSINE/BETA-ALANINE | 3.30E+05 | 4.57E+05 | 7.24E−01 |
| GLUCONIC ACID/D-GULONIC | 6.61E+05 | 9.24E+05 | 7.16E−01 |
| ACID GAMA-LACTONE | | | |
| ASCORBIC ACID-2H | 1.87E+04 | 2.63E+04 | 7.11E−01 |
| 12(S)-HETE | 4.17E+03 | 5.93E+03 | 7.03E−01 |
| CREATINE | 4.62E+05 | 6.62E+05 | 6.98E−01 |
| 12(S)-HETE | 4.17E+03 | 6.71E+03 | 6.22E−01 |
| Taurochenodesoxycholic acid (T- | 2.35E+03 | 3.81E+03 | 6.18E−01 |
| CDCA) | | | |
| GLYCEROL 2- | 3.53E+05 | 6.07E+05 | 5.82E−01 |
| PHOSPHATE/SN-GLYCEROL | | | |
| 3-PHOSPHATE | | | |
| CARNOSINE | 6.58E+04 | 1.14E+05 | 5.76E−01 |
| SARCOSINE/BETA-ALANINE | 2.67E+05 | 4.67E+05 | 5.72E−01 |
| D-GLUCURONIC ACID/D- | 1.76E+05 | 3.42E+05 | 5.14E−01 |
| GLUCURONOLACTONE/D-(+)- | | | |
| GALACTURONIC ACID | | | |
| GLUTATHIONE | 1.65E+06 | 4.08E+06 | 4.03E−01 |
| CYTIDINE | 4.10E+04 | 2.19E+05 | 1.87E−01 |

### Example 6: G4 Structure Formation and/or oxoG Formation Serve as Biomarkers for Cellular ROS Levels

As shown in FIGS. 13A and 13B, G4 DNA structure formation is positively correlated with cellular ROS levels. As shown in FIG. 15A, treatment of cells with high ROS levels with glutathione reduced ethyl ester (GSH), reduces G4 DNA structure formation. As shown in FIGS. 16A and 16B, oxoG formation is also positively correlated with cellular ROS levels. Accordingly, these results demonstrate that levels G4 DNA structure formation and/or oxoG formation in donor somatic cells may serve as a biomarker for elevated cellular ROS levels identifying the cells for treatment before, during, and/or after iPSC reprogramming.

### Example 7: Transcriptome Analysis Reveals Gene Targets for Modulation Cellular ROS Levels

Transcriptome analysis has revealed a number of genes as differentially expressed (more than 5-fold) between high ROS and low ROS fibroblasts. The 26 genes shown in FIG. 18 have been identified as potential genes upstream of ROS.

As shown in FIGS. 19A and 19B, shRNA infection targeting one of these genes, CHI3L1, demonstrates a significant reduction in ROS in the treated high ROS fibroblasts relative to an untreated high ROS fibroblasts.

Accordingly, these results demonstrate that targeting genes implicated in cellular ROS modulation may be useful in methods for generating iPSCs characterized by one or more of increased genomic stability, increased DNA damage response, increased ZSCAN10 expression, and reduced glutathione synthetase (GSS) expression compared to iPSCs produced from untreated control somatic cells grown under similar conditions. In addition, these results demonstrate that donor cells exhibiting altered expression levels in one or more of the differentially expressed genes may identify those cells as candidates for treatment with glutathione or derivatives thereof before, during, or after reprogramming.

### REFERENCES

1. Mandai M, Watanabe A, Kurimoto Y, Hirami Y, Morinaga C, Daimon T, Fujihara M, Akimaru H, Sakai N, Shibata Y, Terada M, Nomiya Y, Tanishima S, Nakamura M, Kamao H, Sugita S, Onishi A, Ito T, Fujita K, Kawamata S, Go M J, Shinohara C, Hata K I, Sawada M, Yamamoto M, Ohta S, Ohara Y, Yoshida K, Kuwahara J, Kitano Y, Amano N, Umekage M, Kitaoka F, Tanaka A, Okada C, Takasu N, Ogawa S, Yamanaka S, Takahashi M. Autologous Induced Stem-Cell-Derived Retinal Cells for Macular Degeneration. N Engl J Med. 2017; 376(11):1038-46. doi: 10.1056/NEJMoa1608368. PubMed PMID: 28296613.
2. Leng T. Stem Cell Treatments: What's Happening in 2015. Review of Ophthalmology. 2015.
3. Garber K. RIKEN suspends first clinical trial involving induced pluripotent stem cells. Nature biotechnology. 2015; 33(9):890-1. doi: 10.1038/nbt0915-890. PubMed PMID: 26348942.
4. Prigione A, Hossini A M, Lichtner B, Serin A, Fauler B, Megges M, Lurz R, Lehrach H, Makrantonaki E, Zouboulis C C, Adjaye J. Mitochondrial-associated cell death mechanisms are reset to an embryonic-like state in aged donor-derived iPS cells harboring chromosomal aberrations. PloS one. 2011; 6(11):e27352. Epub 2011/11/24. doi: 10.1371/journal.pone.0027352. PubMed PMID: 22110631; PMCID: 3215709.
5. Kang E, Wang X, Tippner-Hedges R, Ma H, Folmes C D, Gutierrez N M, Lee Y, Van Dyken C, Ahmed R, Li Y, Koski A, Hayama T, Luo S, Harding C O, Amato P, Jensen J, Battaglia D, Lee D, Wu D, Terzic A, Wolf D P, Huang T, Mitalipov S. Age-Related Accumulation of Somatic Mitochondrial DNA Mutations in Adult-Derived Human iPSCs. Cell stem cell. 2016; 18(5):625-36. doi: 10.1016/j.stem.2016.02.005. PubMed PMID: 27151456.
6. Lo Sardo V, Ferguson W, Erikson G A, Topol E J, Baldwin K K, Torkamani A. Influence of donor age on induced pluripotent stem cells. Nature biotechnology. 2016. doi: 10.1038/nbt.3749. PubMed PMID: 27941802.
7. Coghlan A. Unexpected mutations put stem cell trial on hold. New Sci. 2015; 227(3033):9-. PubMed PMID: WOS:000359512900003.
8. Kang E, Wu J, Gutierrez N M, Koski A, Tippner-Hedges R, Agaronyan K, Platero-Luengo A, Martinez-Redondo P, Ma H, Lee Y, Hayama T, Van Dyken C, Wang X, Luo S, Ahmed R, Li Y, Ji D, Kayali R, Cinnioglu C, Olson S, Jensen J, Battaglia D, Lee D, Wu D, Huang T, Wolf D P, Temiakov D, Belmonte J C, Amato P, Mitalipov S. Mitochondrial replacement in human oocytes carrying pathogenic mitochondrial DNA mutations. Nature. 2016; 540(7632):270-5. doi: 10.1038/nature20592. PubMed PMID: 27919073.
9. Salzano S, Checconi P, Hanschmann E M, Lillig C H, Bowler L D, Chan P, Vaudry D, Mengozzi M, Coppo L, Sacre S, Atkuri K R, Sahaf B, Herzenberg L A, Herzenberg L A, Mullen L, Ghezzi P. Linkage of inflammation and oxidative stress via release of glutathionylated peroxiredoxin-2, which acts as a danger signal. Proc Natl Acad Sci USA. 2014; 111(33):12157-62. doi: 10.1073/pnas.1401712111. PubMed PMID: 25097261; PMCID: PMC4143057.
10. Pastor M D, Nogal A, Molina-Pinelo S, Melendez R, Salinas A, Gonzalez De la Pena M, Martin-Juan J, Corral J, Garcia-Carbonero R, Carnero A, Paz-Ares L. Identification of proteomic signatures associated with lung cancer and COPD. J Proteomics. 2013; 89:227-37. doi: 10.1016/j.jprot.2013.04.037. PubMed PMID: 23665002.
11. Finkel T, Holbrook N J. Oxidants, oxidative stress and the biology of ageing. Nature. 2000; 408(6809):239-47. doi: 10.1038/35041687. PubMed PMID: 11089981.
12. Shaw P X, Werstuck G, Chen Y. Oxidative stress and aging diseases. Oxidative medicine and cellular longevity. 2014; 2014:569146. doi: 10.1155/2014/569146. PubMed PMID: 24959310; PMCID: 4052780.
13. Holmstrom K M, Finkel T. Cellular mechanisms and physiological consequences of redox-dependent signalling. Nature reviews Molecular cell biology. 2014; 15(6):411-21. doi: 10.1038/nrm3801. PubMed PMID: 24854789.
14. Bigarella C L, Liang R, Ghaffari S. Stem cells and the impact of ROS signaling. Development. 2014; 141(22):4206-18. doi: 10.1242/dev.107086. PubMed PMID: 25371358; PMCID: 4302918.
15. Liochev S I. Reactive oxygen species and the free radical theory of aging. Free radical biology & medicine. 2013; 60:1-4. doi: 10.1016/j.freeradbiomed.2013.02.011. PubMed PMID: 23434764.
16. Baccelli I, Trumpp A. The evolving concept of cancer and metastasis stem cells. The Journal of cell biology. 2012; 198(3):281-93. doi: 10.1083/jcb.201202014. PubMed PMID: 22869594; PMCID: 3413352.
17. Alfadda A A, Sallam R M. Reactive oxygen species in health and disease. Journal of biomedicine & biotechnology. 2012; 2012:936486. doi: 10.1155/2012/936486. PubMed PMID: 22927725; PMCID: 3424049.
18. Sleigh M J. The mechanism of DNA breakage by phleomycin in vitro. Nucleic acids research. 1976; 3(4):891-901. Epub 1976/04/01. PubMed PMID: 818624; PMCID: 342953.
19. Franco R, Cidlowski J A. Apoptosis and glutathione: beyond an antioxidant. Cell death and differentiation. 2009; 16(10):1303-14. doi: 10.1038/cdd.2009.107. PubMed PMID: 19662025.
20. Harris I S, Treloar A E, Inoue S, Sasaki M, Gorrini C, Lee K C, Yung K Y, Brenner D, Knobbe-Thomsen C B, Cox M A, Elia A, Berger T, Cescon D W, Adeoye A, Brustle A, Molyneux S D, Mason J M, Li W Y, Yamamoto K, Wakeham A, Berman H K, Khokha R, Done S J, Kavanagh T J, Lam C W, Mak T W. Glutathione and thioredoxin antioxidant pathways synergize to drive cancer initiation and progression. Cancer cell. 2015; 27(2):211-22. doi: 10.1016/j.cce11.2014.11.019. PubMed PMID: 25620030.
21. Guo Z, Kozlov S, Lavin M F, Person M D, Paull T T. ATM activation by oxidative stress. Science. 2010; 330(6003):517-21. Epub 2010/10/23. doi: 10.1126/science.1192912. PubMed PMID: 20966255.
22. Takahashi K, Yamanaka S. Induction of pluripotent stem cells from mouse embryonic and adult fibroblast cultures by defined factors. Cell. 2006; 126(4):663-76. Epub 2006/08/15. doi: 10.1016/j.ce11.2006.07.024. PubMed PMID: 16904174.
23. Smith K P, Luong M X, Stein G S. Pluripotency: toward a gold standard for human ES and iPS cells. J Cell Physiol. 2009; 220(1):21-9. Epub 2009/03/28. doi: 10.1002/jcp.21681. PubMed PMID: 19326392.
24. Kim K, Doi A, Wen B, Ng K, Zhao R, Cahan P, Kim J, Aryee M J, Ji H, Ehrlich L I, Yabuuchi A, Takeuchi A, Cunniff K C, Hongguang H, McKinney-Freeman S, Naveiras O, Yoon T J, Irizarry R A, Jung N, Seita J, Hanna J, Murakami P, Jaenisch R, Weissleder R, Orkin S H, Weissman I L, Feinberg A P, Daley G Q. Epigenetic memory in induced pluripotent stem cells. Nature. 2010; 467(7313):285-90. Epub 2010/07/21. doi: 10.1038/nature09342. PubMed PMID: 20644535; PMCID: 3150836.
25. Liu J C, Lerou P H, Lahav G. Stem cells: balancing resistance and sensitivity to DNA damage. Trends in cell biology. 2014; 24(5):268-74. doi: 10.1016/j.tcb.2014.03.002. PubMed PMID: 24721782; PMCID: 4342985.
26. Liu J C, Guan X, Ryan J A, Rivera A G, Mock C, Agrawal V, Letai A, Lerou P H, Lahav G. High mitochondrial priming sensitizes hESCs to DNA-damage-induced apoptosis. Cell stem cell. 2013; 13(4):483-91. doi: 10.1016/j.stem.2013.07.018. PubMed PMID: 23954752; PMCID: 4109647.
27. Zhivotovsky B, Kroemer G. Apoptosis and genomic instability. Nature reviews Molecular cell biology. 2004; 5(9):752-62. doi: 10.1038/nrm1443. PubMed PMID: 15340382.
28. Gurdon J B, Wilmut I. Nuclear transfer to eggs and oocytes. Cold Spring Harbor perspectives in biology. 2011; 3(6). doi: 10.1101/cshperspect.a002659. PubMed PMID: 21555407; PMCID: 3098674.
29. Ma H, Morey R, O'Neil R C, He Y, Daughtry B, Schultz M D, Hariharan M, Nery J R, Castanon R, Sabatini K, Thiagarajan R D, Tachibana M, Kang E, Tippner-Hedges R, Ahmed R, Gutierrez N M, Van Dyken C, Polat A, Sugawara A, Sparman M, Gokhale S, Amato P, Wolf D P, Ecker J R, Laurent L C, Mitalipov S. Abnormalities in human pluripotent cells due to reprogramming mechanisms. Nature. 2014; 511(7508):177-83. doi: 10.1038/nature13551. PubMed PMID: 25008523.
30. Cowan C A, Atienza J, Melton D A, Eggan K. Nuclear reprogramming of somatic cells after fusion with human embryonic stem cells. Science. 2005; 309(5739):1369-73. Epub 2005/08/27. doi: 10.1126/science.1116447. PubMed PMID: 16123299.
31. Kim J, Chu J, Shen X, Wang J, Orkin S H. An extended transcriptional network for pluripotency of embryonic stem cells. Cell. 2008; 132(6):1049-61. Epub 2008/03/25. doi: 10.1016/j.ce11.2008.02.039. PubMed PMID: 18358816; PMCID: 3837340.
32. Polo S E, Jackson S P. Dynamics of DNA damage response proteins at DNA breaks: a focus on protein modifications. Genes Dev. 2011; 25(5):409-33. Epub 2011/03/03. doi: 10.1101/gad.2021311. PubMed PMID: 21363960; PMCID: 3049283.
33. Yu H B, Kunarso G, Hong F H, Stanton L W. Zfp206, Oct4, and Sox2 are integrated components of a transcriptional regulatory network in embryonic stem cells. The Journal of biological chemistry. 2009; 284(45):31327-35. Epub 2009/09/11. doi: 10.1074/jbc.M109.016162. PubMed PMID: 19740739; PMCID: 2781530.
34. Ford J, Ahmed S, Allison S, Jiang M, Milner J. JNK2-dependent regulation of SIRT1 protein stability. Cell Cycle. 2008; 7(19):3091-7. Epub 2008/10/08. PubMed PMID: 18838864.
35. Jiang J, Lv W, Ye X, Wang L, Zhang M, Yang H, Okuka M, Zhou C, Zhang X, Liu L, Li J. Zscan4 promotes genomic stability during reprogramming and dramatically improves the quality of iPS cells as demonstrated by tetraploid complementation. Cell research. 2013; 23(1):92-106. doi: 10.1038/cr.2012.157. PubMed PMID: 23147797; PMCID: 3541664.
36. Zhang W, Walker E, Tamplin O J, Rossant J, Stanford W L, Hughes T R. Zfp206 regulates ES cell gene expression and differentiation. Nucleic acids research. 2006; 34(17):4780-90. PubMed PMID: 16971461.
37. Chan E M, Ratanasirintrawoot S, Park I H, Manos P D, Loh Y H, Huo H, Miller J D, Hartung O, Rho J, Ince T A, Daley G Q, Schlaeger T M. Live cell imaging distinguishes bona fide human iPS cells from partially reprogrammed cells. Nature biotechnology. 2009. PubMed PMID: 19826408.
38. Tsuda H, Sasaki K, Tanaka N. Establishment of hypoxanthine phosphoribosyltransferase(HPRT)-locus mutation assay system in mouse ES cells. Alt Animal Testing Exp. 2005; 11(2):118-28.
39. Miller J D, Ganat Y M, Kishinevsky S, Bowman R L, Liu B, Tu E Y, Mandal P K, Vera E, Shim J W, Kriks S, Taldone T, Fusaki N, Tomishima M J, Krainc D, Milner T A, Rossi D J, Studer L. Human iPSC-based modeling of late-onset disease via progerin-induced aging. Cell stem cell. 2013; 13(6):691-705. doi: 10.1016/j.stem.2013.11.006. PubMed PMID: 24315443; PMCID: 4153390.
40. Kyttala A, Moraghebi R, Valensisi C, Kettunen J, Andrus C, Pasumarthy K K, Nakanishi M, Nishimura K, Ohtaka M, Weltner J, Van Handel B, Parkkonen O, Sinisalo J, Jalanko A, Hawkins R D, Woods N B, Otonkoski T, Trokovic R. Genetic Variability Overrides the Impact of Parental Cell Type and Determines iPSC Differentiation Potential. Stem cell reports. 2016; 6(2):200-12. doi: 10.1016/j.stemcr.2015.12.009. PubMed PMID: 26777058.
41. Baslan T, Kendall J, Ward B, Cox H, Leotta A, Rodgers L, Riggs M, D'Italia S, Sun G, Yong M, Miskimen K, Gilmore H, Saborowski M, Dimitrova N, Krasnitz A, Harris L, Wigler M, Hicks J. Optimizing sparse sequencing of single cells for highly multiplex copy number profiling. Genome research. 2015; 25(5):714-24. doi: 10.1101/gr.188060.114. PubMed PMID: 25858951; PMCID: 4417119.
42. Takahashi K, Tanabe K, Ohnuki M, Narita M, Ichisaka T, Tomoda K, Yamanaka S. Induction of pluripotent stem cells from adult human fibroblasts by defined factors. Cell. 2007; 131(5):861-72. doi: 10.1016/j.ce11.2007.11.019. PubMed PMID: 18035408.
43. Nakagawa M, Koyanagi M, Tanabe K, Takahashi K, Ichisaka T, Aoi T, Okita K, Mochiduki Y, Takizawa N, Yamanaka S. Generation of induced pluripotent stem cells without Myc from mouse and human fibroblasts. Nature biotechnology. 2008; 26(1):101-6. doi: 10.1038/nbt1374. PubMed PMID: 18059259.
44. Araki R, Hoki Y, Uda M, Nakamura M, Jincho Y, Tamura C, Sunayama M, Ando S, Sugiura M, Yoshida M A, Kasama Y, Abe M. Crucial role of c-Myc in the generation of induced pluripotent stem cells. Stem cells. 2011; 29(9):1362-70. doi: 10.1002/stem.685. PubMed PMID: 21732496.
45. Okita K, Matsumura Y, Sato Y, Okada A, Morizane A, Okamoto S, Hong H, Nakagawa M, Tanabe K, Tezuka K, Shibata T, Kunisada T, Takahashi M, Takahashi J, Saji H, Yamanaka S. A more efficient method to generate integration-free human iPS cells. Nature methods. 2011; 8(5):409-12. doi: 10.1038/nmeth.1591. PubMed PMID: 21460823.
46. Unternaehrer J J, Zhao R, Kim K, Cesana M, Powers J T, Ratanasirintrawoot S, Onder T, Shibue T, Weinberg R A, Daley G Q. The epithelial-mesenchymal transition factor SNAIL paradoxically enhances reprogramming. Stem cell reports. 2014; 3(5):691-8. doi: 10.1016/j.stemcr.2014.09.008. PubMed PMID: 25316190; PMCID: 4235745.
47. Yamamoto S, Nagao Y, Kuroiwa K, Hakamata Y, Ichida M, Saito-Ohara F, Tominaga K, Endo H. Rapid selection of XO embryonic stem cells using Y chromosome-linked GFP transgenic mice. Transgenic research. 2014; 23(5):757-65. doi: 10.1007/s11248-014-9813-0. PubMed PMID: 25008421.
48. Seol H W, Oh S K, Park Y B, Kim H S, Baek J A, Seo J, Kim E H, Ku S Y, Kim S H, Choi Y M, Moon S Y. Separation and maintenance of normal cells from human embryonic stem cells with trisomy 12 mosaicism. Chromosome research: an international journal on the molecular, supramolecular and evolutionary aspects of chromosome biology. 2008; 16(8):1075-84. doi: 10.1007/510577-008-1258-y. PubMed PMID: 18937039.
49. Hollander M, Wolfe D A. Nonparametric statistical methods. New York: Wiley; 1973. xviii, 503 p. p.
50. Hong H, Takahashi K, Ichisaka T, Aoi T, Kanagawa O, Nakagawa M, Okita K, Yamanaka S. Suppression of induced pluripotent stem cell generation by the p53-p21 pathway. Nature. 2009; 460(7259):1132-5. PubMed PMID: 19668191.
51. Li H, Collado M, Villasante A, Strati K, Ortega S, Canamero M, Blasco M A, Serrano M. The Ink4/Arf locus is a barrier for iPS cell reprogramming. Nature. 2009; 460(7259):1136-9. PubMed PMID: 19668188.
52. Utikal J, Polo J M, Stadtfeld M, Maherali N, Kulalert W, Walsh R M, Khalil A, Rheinwald J G, Hochedlinger K. Immortalization eliminates a roadblock during cellular reprogramming into iPS cells. Nature. 2009; 460(7259):1145-8. PubMed PMID: 19668190.
53. Son J, Lyssiotis C A, Ying H, Wang X, Hua S, Ligorio M, Perera R M, Ferrone C R, Mullarky E, Shyh-Chang N, Kang Y, Fleming J B, Bardeesy N, Asara J M, Haigis M C, DePinho R A, Cantley L C, Kimmelman A C. Glutamine supports pancreatic cancer growth through a KRAS-regulated metabolic pathway. Nature. 2013; 496(7443):101-5. doi: 10.1038/nature12040. PubMed PMID: 23535601; PMCID: 3656466.
54. Balendiran G K, Dabur R, Fraser D. The role of glutathione in cancer. Cell biochemistry and function. 2004; 22(6):343-52. doi: 10.1002/cbf 1149. PubMed PMID: 15386533.
55. Estrela J M, Ortega A, Obrador E. Glutathione in cancer biology and therapy. Critical reviews in clinical laboratory sciences. 2006; 43(2):143-81. doi: 10.1080/10408360500523878. PubMed PMID: 16517421.
56. Traverso N, Ricciarelli R, Nitti M, Marengo B, Furfaro A L, Pronzato M A, Marinari U M, Domenicotti C. Role of glutathione in cancer progression and chemoresistance. Oxidative medicine and cellular longevity. 2013; 2013:972913. doi: 10.1155/2013/972913. PubMed PMID: 23766865; PMCID: 3673338.
57. An G, Xu Y, Shi L, Shizhen Z, Deng S, Xie Z, Sui W, Zhan F, Qiu L. Chromosome 1q21 gains confer inferior outcomes in multiple myeloma treated with bortezomib but copy number variation and percentage of plasma cells involved have no additional prognostic value. Haematologica. 2014; 99(2):353-9. doi: 10.3324/haematol.2013.088211. PubMed PMID: 24213147; PMCID: 3912967.
58. Pezzolo A, Rossi E, Gimelli S, Parodi F, Negri F, Conte M, Pistorio A, Sementa A, Pistoia V, Zuffardi O, Gambini C. Presence of 1q gain and absence of 7p gain are new predictors of local or metastatic relapse in localized resectable neuroblastoma. Neuro-oncology. 2009; 11(2):192-200. doi: 10.1215/15228517-2008-086. PubMed PMID: 18923191; PMCID: 2718991.
59. Lo K C, Ma C, Bundy B N, Pomeroy S L, Eberhart C G, Cowell J K. Gain of 1q is a potential univariate negative prognostic marker for survival in medulloblastoma. Clinical cancer research: an official journal of the American Association for Cancer Research. 2007; 13(23):7022-8. doi: 10.1158/1078-0432.CCR-07-1420. PubMed PMID: 18056178.
60. Zhou G, Meng S, Li Y, Ghebre Y T, Cooke J P. Optimal ROS Signaling Is Critical for Nuclear Reprogramming. Cell reports. 2016; 15(5):919-25. doi: 10.1016/j.celrep.2016.03.084. PubMed PMID: 27117405; PMCID: 4856580.
61. Brock A, Krause S, Li H, Kowalski M, Goldberg M S, Collins J J, Ingber D E. Silencing HoxA1 by intraductal injection of siRNA lipidoid nanoparticles prevents mammary tumor progression in mice. Sci Transl Med. 2014; 6(217):217ra2. doi: 10.1126/scitranslmed.3007048. PubMed PMID: 24382894.
62. Feller C, Forne I, Imhof A, Becker P B. Global and specific responses of the histone acetylome to systematic perturbation. Molecular cell. 2015; 57(3):559-71. doi: 10.1016/j.molce1.2014.12.008. PubMed PMID: 25578876.
63. Yuan Z F, Lin S, Molden R C, Cao X J, Bhanu N V, Wang X, Sidoli S, Liu S, Garcia B A. EpiProfile Quantifies Histone Peptides With Modifications by Extracting Retention Time and Intensity in High-resolution Mass Spectra. Mol Cell Proteomics. 2015; 14(6):1696-707. doi: 10.1074/mcp.M114.046011. PubMed PMID: 25805797; PMCID: PMC4458730.
64. Cahan P, Li H, Morris S A, Lummertz da Rocha E, Daley G Q, Collins J J. CellNet: network biology applied to stem cell engineering. Cell. 2014; 158(4):903-15. doi: 10.1016/j.ce11.2014.07.020. PubMed PMID: 25126793; PMCID: PMC4233680.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. A method of producing induced pluripotent stem cells (iPSCs) from mammalian non-pluripotent cells, wherein the iPSCs are characterized by one or more of increased genomic stability, increased DNA damage response, increased ZSCAN10 expression, and reduced glutathione synthetase (GSS) expression, the method comprising:
culturing non-pluripotent cells treated with an effective amount of glutathione or derivatives thereof prior to the initiation of reprogramming, during reprogramming, and/or after reprogramming of the non-pluripotent cells under conditions that allow for the production of iPSCs,
thereby producing iPSCs with one or more of increased genomic stability, increased DNA damage response, increased ZSCAN10 expression, and reduced GSS expression as compared to iPSCs produced from untreated control non-pluripotent cells grown under similar conditions.

2. The method of claim 1 further comprising identifying non-pluripotent cells for treatment with glutathione or derivatives thereof, wherein the non-pluripotent cells identified for treatment express an elevated cellular reactive oxygen species (ROS) level prior to treatment relative to that observed in untreated control non-pluripotent cells, wherein the elevated cellular ROS level identifies the non-pluripotent cells for treatment with glutathione or derivatives thereof and the lack of the elevated cellular ROS level does not identify the non-pluripotent cells for treatment with glutathione or derivatives thereof.

3. The method of claim 1, wherein the efficiency of reprogramming the non-pluripotent cells treated with glutathione or derivatives thereof is increased relative to untreated control non-pluripotent cells.

4. The method of claim 3, wherein treatment with the glutathione or derivatives thereof increases the efficiency of reprogramming the non-pluripotent cells into iPSCs by at least 10-fold relative to untreated control non-pluripotent cells.

5. The method of claim 1, wherein treatment with glutathione or derivatives thereof restores ZSCAN10 expression levels in iPSCs to about 50% or more of the respective expression levels of embryonic stem cells (ESCs).

6. The method of claim 1, wherein the mammalian non-pluripotent cells are somatic cells.

7. The method of claim 6, wherein the somatic cells are aged somatic cells.

8. The method of claim 6, wherein the somatic cells are somatic cells from an embryonic stage.

9. The method of claim 6, wherein the somatic cells express an increased cellular ROS level relative to that observed in young somatic cells.

10. The method of claim 6, wherein the somatic cells are incapable of generating iPSCs.

11. The method of claim 6, wherein the somatic cells are selected from the group consisting of: fibroblast cells, cells from blood, cells from ocular tissue, epithelial cells, osteocytes, chondrocytes, neurons, muscle cells, hepatic cells, intestinal cells, spleen cells, adult stem cells, and progenitor cells from adult stem cells.

12. The method of claim 1, wherein the mammalian non-pluripotent cells are progenitor cells.

13. Induced pluripotent stem cells (iPSCs) produced by the method of claim 1, wherein the iPSCs produced from the non-pluripotent cells treated with glutathione or derivatives thereof are characterized by one or more of increased genomic stability, increased DNA damage response, increased iPSC reprogramming efficiency, increased ZSCAN10 expression, and reduced GSS expression as compared to iPSCs produced from untreated control non-pluripotent cells grown under similar conditions.

14. iPSCs produced by the method of claim 1, wherein the iPSCs are characterized by increased genomic stability as compared to iPSCs produced from untreated control non-pluripotent cells grown under similar conditions.

15. iPSCs produced by the method of claim 1, wherein the iPSCs are characterized by increased DNA damage response as compared to iPSCs produced from untreated control non-pluripotent cells grown under similar conditions.

16. iPSCs produced by the method of claim 1, wherein the iPSCs are characterized by increased ZSCAN10 expression as compared to iPSCs produced from untreated control non-pluripotent cells grown under similar conditions.

17. iPSCs produced by the method of claim 1, wherein the iPSCs are characterized by reduced GSS expression as compared to iPSCs produced from untreated control non-pluripotent cells grown under similar conditions.

18. iPSCs produced by the method of claim 1, wherein the iPSCs are characterized by increased iPSC reprogramming efficiency as compared to iPSCs produced from untreated control non-pluripotent cells grown under similar conditions.

19. The method of claim 1, wherein the glutathione is glutathione reduced ethyl ester.

20. A method of producing induced pluripotent stem cells derived from aged somatic cells (A-iPSCs) having one or more of increased genomic stability, increased DNA damage response, increased ZSCAN10 expression, and reduced glutathione synthetase (GSS) expression, the method comprising:
culturing aged somatic cells treated with an effective amount of glutathione or derivatives thereof prior to the initiation of reprogramming, during reprogramming, and/or after reprogramming of the aged somatic cells under conditions that allow for the production of A-iPSCs,
thereby producing A-iPSCs with one or more of increased genomic stability, increased DNA damage response, increased iPSC reprogramming efficiency, increased ZSCAN10 expression, and reduced GSS expression as compared to that observed in A-iPSCs produced from untreated control aged somatic cells grown under similar conditions and/or comparable to that observed in young iPSCs (Y-iPSCs) or ESCs.

21. The method of claim 20 further comprising identifying aged somatic cells for treatment with glutathione or derivatives thereof, wherein the aged somatic cells identified for treatment express an elevated cellular reactive oxygen species (ROS) level prior to treatment relative to one or more of untreated control aged somatic cells, young somatic cells, and ESCs, wherein the elevated cellular ROS level identifies the aged somatic cells for treatment with glutathione or derivatives thereof and the lack of the elevated cellular ROS level does not identify the aged somatic cells for treatment with glutathione or derivatives thereof.

22. A-iPSCs produced by the method of claim 20, wherein the A-iPSCs produced from the aged somatic cells treated with glutathione or derivatives thereof are characterized by one or more of increased genomic stability, increased DNA damage response, increased iPSC reprogramming efficiency, increased ZSCAN10 expression, and reduced GSS expression as compared to A-iPSCs produced from untreated control aged somatic cells grown under similar conditions and/or comparable to that observed in young iPSCs (Y-iPSCs) or ESCs.

23. A-iPSCs produced by the method of claim 20, wherein the A-iPSCs are characterized by increased genomic stability as compared to A-iPSCs produced from untreated control aged somatic cells grown under similar conditions and/or comparable to that observed in young iPSCs (Y-iPSCs) or ESCs.

24. A-iPSCs produced by the method of claim 20, wherein the A-iPSCs are characterized by increased DNA damage response as compared to A-iPSCs produced from untreated control aged somatic cells grown under similar conditions and/or comparable to that observed in young iPSCs (Y-iPSCs) or ESCs.

25. A-iPSCs produced by the method of claim 20, wherein the A-iPSCs are characterized by increased iPSC reprogramming efficiency as compared to A-iPSCs produced from untreated control aged somatic cells grown under similar conditions and/or comparable to that observed in young iPSCs (Y-iPSCs) or ESCs.

26. A-iPSCs produced by the method of claim 20, wherein the A-iPSCs are characterized by increased ZSCAN10 expression as compared to A-iPSCs produced from untreated control aged somatic cells grown under similar conditions and/or comparable to that observed in young iPSCs (Y-iPSCs) or ESCs.

27. A-iPSCs produced by the method of claim 20, wherein the A-iPSCs are characterized by reduced glutathione synthetase (GSS) expression as compared to A-iPSCs produced from untreated control aged somatic cells grown under similar conditions and/or comparable to that observed in young iPSCs (Y-iPSCs) or ESCs.

28. The method of claim 20, wherein the glutathione is glutathione reduced ethyl ester.

29. A method of producing pluripotent stem cells including embryonic stem cell derivation from blastocyst, parthenogenetic ES cells, nuclear transferred ES cells to improve genomic stability, derivation efficiency, and reprogramming quality comprising:
culturing embryos treated with an effective amount of glutathione or derivatives thereof prior to the initiation of reprogramming and/or during reprogramming of the embryos under conditions that allow for the production of ES cells, parthenogenetic ES cells, nuclear transferred ES cells to minimize the oxidative stress (ROS)-mediated inhibitory effects during reprogramming of the pluripotent stem cells,
thereby producing pluripotent stem cells with one or more of improved genomic stability, improved DNA damage response, reprogramming quality with increased pluripotent gene expression including ZSCAN10 expression and reduced GSS expression as compared to the pluripotent stem cells produced from untreated control cells grown under similar conditions.

30. A method for stem cell therapy comprising:
(a) isolating a non-pluripotent cell from a subject;
(b) producing an iPSC by the method of claim 1;
(c) differentiating the iPSC ex vivo into a differentiated cell; and
(d) administering the differentiated cell to the subject.

31. A method for stem cell therapy comprising:
(a) isolating an aged somatic cell from a subject;
(b) producing an A-iPSC by the method of claim 20;
(c) differentiating the A-iPSC ex vivo into a differentiated cell; and
(d) administering the differentiated cell to the subject.

32. The method of claim 2, wherein the elevated cellular ROS level of the non-pluripotent cells identified for treatment is defined by a metabolic profile comprising one or more metabolites exhibiting increased levels relative to that observed in untreated control non-pluripotent cells.

33. The method of claim 32, wherein the one or more metabolites exhibiting increased levels is selected from the group consisting of adenosine, cytidine, xanthine, and cytidine 3′ monophosphate (3′-CMP).

34. The method of claim 2, wherein the elevated cellular ROS level of the non-pluripotent cells identified for treatment is defined by an increased gene expression level of one or more genes selected from the group consisting of ST6GALNAC6, IGFBP5, PDGFD, SURF4, BOC, ADGRD1, MPDU1, RPS4Y1, MME, SET, DOK1, COLEC12, HOXC10, SULF2, ADAMTSL1, ELN, MGRN1, COL15A1, ZEB1, SFRP1, CLDN11, LGALS3BP, CHI3L1, SPG21, PI16, and MCFD2 relative to that observed in untreated control non-pluripotent cells.

35. The method of claim 34, wherein the gene expression level of the one or more genes in the non-pluripotent cells identified for treatment is increased by about 2-fold to about 5-fold relative to that observed in untreated control non-pluripotent cells.

36. The method of claim 34, wherein the gene expression level of the one or more genes in the non-pluripotent cells identified for treatment is increased by about 5-fold relative to that observed in untreated control non-pluripotent cells.

37. The method of claim 2, wherein the elevated cellular ROS level of the non-pluripotent cells identified for treatment is defined by an increased cellular G-quadruplex (G4) DNA structure formation relative to that observed in untreated control non-pluripotent cells.

38. The method of claim 37, wherein the G4 DNA structure formation in the non-pluripotent cells identified for treatment is increased by about 2-fold relative to that observed in untreated control non-pluripotent cells.

39. The method of claim 2, wherein the elevated cellular ROS level of the non-pluripotent cells identified for treatment is defined by an increased 8-oxo-guanine (oxoG) formation relative to that observed in untreated control non-pluripotent cells.

40. The method of claim 39, wherein the oxoG formation in the non-pluripotent cells identified for treatment is increased by about 2-fold to about 3-fold relative to that observed in untreated control non-pluripotent cells.

41. The method of claim 21, wherein the elevated cellular ROS level of the aged somatic cells identified for treatment is defined by a metabolic profile comprising one or more metabolites exhibiting increased levels relative to that observed in one or more of untreated control aged somatic cells, young somatic cells, and ESCs.

42. The method of claim 41, wherein the one or more metabolites exhibiting increased levels is selected from the group consisting of adenosine, cytidine, xanthine, and cytidine 3′ monophosphate (3′-CMP).

43. The method of claim 21, wherein the elevated cellular ROS level of the aged somatic cells identified for treatment is defined by an increased gene expression level of one or more genes selected from the group consisting of ST6GALNAC6, IGFBP5, PDGFD, SURF4, BOC, ADGRD1, MPDU1, RPS4Y1, MME, SET, DOK1, COLEC12, HOXC10, SULF2, ADAMTSL1, ELN, MGRN1, COL15A1, ZEB1, SFRP1, CLDN11, LGALS3BP, CHI3L1, SPG21, PI16, and MCFD2 relative to that observed in one or more of untreated control aged somatic cells, young somatic cells, and ESCs.

44. The method of claim 43, wherein the gene expression level of the one or more genes in the aged somatic cells identified for treatment is increased by about 2-fold to about 5-fold relative to that observed in one or more of untreated control aged somatic cells, young somatic cells, and ESCs.

45. The method of claim 43, wherein the gene expression level of the one or more genes in the aged somatic cells identified for treatment is increased by about 5-fold relative to that observed in one or more of untreated control aged somatic cells, young somatic cells, and ESCs.

46. The method of claim 21, wherein the elevated cellular ROS level of the aged somatic cells identified for treatment is defined by an increased cellular G-quadruplex (G4) DNA structure formation relative to that observed in one or more of untreated control aged somatic cells, young somatic cells, and ESCs.

47. The method of claim 46, wherein the G4 DNA structure formation in the aged somatic cells identified for treatment is increased by about 2-fold relative to that observed in one or more of untreated control aged somatic cells, young somatic cells, and ESCs.

48. The method of claim 21, wherein the elevated cellular ROS level of the aged somatic cells identified for treatment is defined by an increased 8-oxo-guanine (oxoG) formation relative to that observed in one or more of untreated control aged somatic cells, young somatic cells, and ESCs.

49. The method of claim 48, wherein the oxoG formation in the aged somatic cells identified for treatment is increased by about 2-fold to about 3-fold relative to that observed in one or more of untreated control aged somatic cells, young somatic cells, and ESCs.

50. The method of claim 29 further comprising identifying embryonic stem cell derivation from blastocyst, parthenogenetic ES cells, or nuclear transferred ES cells for treatment with glutathione or derivatives thereof, wherein the embryonic stem cell derivation from blastocyst, parthenogenetic ES cells, or nuclear transferred ES cells identified for treatment express an elevated reactive oxygen species (ROS) level prior to treatment relative to one or more of untreated control embryonic stem cell derivation from blastocyst, parthenogenetic ES cells, or nuclear transferred ES cells, wherein the elevated cellular ROS level identifies the embryonic stem cell derivation from blastocyst, parthenogenetic ES cells, or nuclear transferred ES cells for treatment with glutathione or derivatives thereof and the lack of the elevated cellular ROS level does not identify the embryonic stem cell derivation from blastocyst, parthenogenetic ES cells, or nuclear transferred ES cells for treatment with glutathione or derivatives thereof.

51. The method of claim 50, wherein the elevated cellular ROS level of the embryonic stem cell derivation from blastocyst, parthenogenetic ES cells, or nuclear transferred ES cells identified for treatment is defined by a metabolic profile comprising one or more metabolites exhibiting increased levels relative to that observed in one or more of untreated control embryonic stem cell derivation from blastocyst, parthenogenetic ES cells, or nuclear transferred ES cells.

52. The method of claim 51, wherein the one or more metabolites exhibiting increased levels is selected from the group consisting of adenosine, cytidine, xanthine, and cytidine 3′ monophosphate (3′-CMP).

53. The method of claim 51, wherein the elevated cellular ROS level of the embryonic stem cell derivation from blastocyst, parthenogenetic ES cells, or nuclear transferred ES cells identified for treatment is defined by an increased gene expression level of one or more genes selected from the group consisting of ST6GALNAC6, IGFBP5, PDGFD, SURF4, BOC, ADGRD1, MPDU1, RPS4Y1, MME, SET, DOK1, COLEC12, HOXC10, SULF2, ADAMTSL1, ELN, MGRN1, COL15A1, ZEB1, SFRP1, CLDN11, LGALS3BP, CHI3L1, SPG21, PI16, and MCFD2 relative to that observed in one or more of untreated control embryonic stem cell derivation from blastocyst, parthenogenetic ES cells, or nuclear transferred ES cells.

54. The method of claim 53, wherein the gene expression level of the one or more genes in the embryonic stem cell derivation from blastocyst, parthenogenetic ES cells, or nuclear transferred ES cells identified for treatment is increased by about 2-fold to about 5-fold relative to that observed in one or more of untreated control embryonic stem cell derivation from blastocyst, parthenogenetic ES cells, or nuclear transferred ES cells.

55. The method of claim 53, wherein the gene expression level of the one or more genes in the embryonic stem cell derivation from blastocyst, parthenogenetic ES cells, or nuclear transferred ES cells identified for treatment is increased by about 5-fold relative to that observed in one or more of untreated control embryonic stem cell derivation from blastocyst, parthenogenetic ES cells, or nuclear transferred ES cells.

56. The method of claim 51, wherein the elevated cellular ROS level of the embryonic stem cell derivation from blastocyst, parthenogenetic ES cells, or nuclear transferred ES cells identified for treatment is defined by an increased cellular G-quadruplex (G4) DNA structure formation relative to that observed in one or more of untreated control embryonic stem cell derivation from blastocyst, parthenogenetic ES cells, or nuclear transferred ES cells.

57. The method of claim 56, wherein the G4 DNA structure formation in the embryonic stem cell derivation from blastocyst, parthenogenetic ES cells, or nuclear transferred ES cells identified for treatment is increased by about 2-fold relative to that observed in one or more of untreated control embryonic stem cell derivation from blastocyst, parthenogenetic ES cells, or nuclear transferred ES cells.

58. The method of claim 51, wherein the elevated ROS level of the embryonic stem cell derivation from blastocyst, parthenogenetic ES cells, or nuclear transferred ES cells identified for treatment is defined by an increased 8-oxo-guanine (oxoG) formation relative to that observed in one or more of untreated control embryonic stem cell derivation from blastocyst, parthenogenetic ES cells, or nuclear transferred ES cells.

59. The method of claim 58, wherein the oxoG formation in the embryonic stem cell derivation from blastocyst, parthenogenetic ES cells, or nuclear transferred ES cells identified for treatment is increased by about 2-fold to about 3-fold relative to that observed in one or more of untreated control embryonic stem cell derivation from blastocyst, parthenogenetic ES cells, or nuclear transferred ES cells.

60. A method for stem cell therapy comprising:
(a) isolating a non-pluripotent cell from a subject;
(b) producing an iPSC by the method of any one of claim 2-12, 19, or 32-40;
(c) differentiating the iPSC ex vivo into a differentiated cell; and
(d) administering the differentiated cell to the subject.

61. A method for stem cell therapy comprising:
(a) isolating an aged somatic cell from a subject;
(b) producing an A-iPSC by the method of any one of claim 21, 28, or 41-49;
(c) differentiating the A-iPSC ex vivo into a differentiated cell; and
(d) administering the differentiated cell to the subject.

62. A kit comprising glutathione reduced ethyl ester, reprogramming factors, and instructions for reprogramming a plurality of non-pluripotent cells.


SEQUENCE LISTING (26 sequences)

SEQ ID NO: 1 - DNA, 20 bp, Artificial Sequence
  Description of Artificial Sequence Synthetic primer
caccgtcaaggctgagaacg

SEQ ID NO: 2 - DNA, 20 bp, Artificial Sequence
  Description of Artificial Sequence Synthetic primer
gccccacttgattttggagg

SEQ ID NO: 3 - DNA, 20 bp, Artificial Sequence
  Description of Artificial Sequence Synthetic primer
ccttactctcaggagcgcag

SEQ ID NO: 4 - DNA, 20 bp, Artificial Sequence
  Description of Artificial Sequence Synthetic primer
tgtgccagagaataaggcgt

SEQ ID NO: 5 - DNA, 20 bp, Artificial Sequence
  Description of Artificial Sequence Synthetic primer
cctcacttcactgcactgta

SEQ ID NO: 6 - DNA, 20 bp, Artificial Sequence
  Description of Artificial Sequence Synthetic primer
caggttttctttccctagct

SEQ ID NO: 7 - DNA, 19 bp, Artificial Sequence
  Description of Artificial Sequence Synthetic primer
cccagcagacttcacatgt

SEQ ID NO: 8 - DNA, 20 bp, Artificial Sequence
  Description of Artificial Sequence Synthetic primer
cctcccatttccctcgtttt

SEQ ID NO: 9 - DNA, 20 bp, Artificial Sequence
  Description of Artificial Sequence Synthetic primer
tgcctcaaattggactttgg

SEQ ID NO: 10 - DNA, 22 bp, Artificial Sequence
  Description of Artificial Sequence Synthetic primer
gattgaaattctgtgtaactgc

SEQ ID NO: 11 - DNA, 20 bp, Artificial Sequence
  Description of Artificial Sequence Synthetic primer
gatgaactgaccaggcacta

SEQ ID NO: 12 - DNA, 20 bp, Artificial Sequence
  Description of Artificial Sequence Synthetic primer
gtgggtcatatccactgtct

SEQ ID NO: 13 - DNA, 20 bp, Artificial Sequence
  Description of Artificial Sequence Synthetic primer
cggccaaaaggaaagagcat

SEQ ID NO: 14 - DNA, 21 bp, Artificial Sequence
  Description of Artificial Sequence Synthetic primer
gcttgcattccttggcatgat

SEQ ID NO: 15 - DNA, 20 bp, Artificial Sequence
  Description of Artificial Sequence Synthetic primer
gagagggaagccccttggat

SEQ ID NO: 16 - DNA, 23 bp, Artificial Sequence
  Description of Artificial Sequence Synthetic primer
actggttctccttcttttaggct

SEQ ID NO: 17 - DNA, 25 bp, Artificial Sequence
  Description of Artificial Sequence Synthetic primer
cagccccgattcttccaccagtccc

SEQ ID NO: 18 - DNA, 25 bp, Artificial Sequence
  Description of Artificial Sequence Synthetic primer
cggaagattcccagtcgggttcacc

SEQ ID NO: 19 - DNA, 27 bp, Homo sapiens
gggtggggaaaaccagggggtgggggg

SEQ ID NO: 20 - DNA, 22 bp, Homo sapiens
gggagggtgaacctgggaaggg

SEQ ID NO: 21 - DNA, 25 bp, Homo sapiens
gggaagtggggtgtggtgggaaggg

SEQ ID NO: 22 - DNA, 19 bp, Homo sapiens
gggaggggaggggaggggg

SEQ ID NO: 23 - DNA, 25 bp, Homo sapiens
ggggaggcatgggatgggaatgggg

SEQ ID NO: 24 - DNA, 27 bp, Homo sapiens
gggaggcgtgggggtgggacggtgggg

SEQ ID NO: 25 - DNA, 24 bp, Homo sapiens
gggacagggagcgggggatggggg

SEQ ID NO: 26 - DNA, 28 bp, Homo sapiens
gggagggcggggcggggcagggcggggg